(19) United States
(12) Patent Application Publication
(10) Pub. No.: US 2024/0124439 A1
(43) Pub. Date: Apr. 18, 2024

(54) Pyrido[2,3-D]Imidazole Derivatives and Their Use As Inhibitors of ITK for the Treatment of Skin Disease

(71) Applicant: Pfizer Inc., New York, NY (US)

(72) Inventors: Scott William Bagley, Voluntown, CT (US); Agustin Casimiro-Garcia, Concord, MA (US); Jennifer Elizabeth Davoren, Cambridge, MA (US); Rajiah Aldrin Denny, Sharon, MA (US); Brian Stephen Gerstenberger, Cambridge, MA (US); Katherine Lin Lee, West Newton, MA (US); Frank Eldridge Lovering, Acton, MA (US); Mihir Dineshkumar Parikh, East Greenwich, RI (US); Joseph Walter Strohbach, Wentzville, MO (US); John Isidro Trujillo, Ledyard, CT (US)

(73) Assignee: Pfizer Inc., New York, NY (US)

(21) Appl. No.: 18/257,195

(22) PCT Filed: Dec. 13, 2021
(86) PCT No.: PCT/IB2021/061645

§ 371 Date: Jun. 13, 2023

Related U.S. Application Data
(60) Provisional application No. 63/125,671, filed on Dec. 15, 2020.

(51) Int. Cl. C07D 471/04; C07D 519/00

(52) U.S. Cl. CPC C07D 471/04; C07D 519/00

(57) ABSTRACT

The invention relates to imidazopyridines of Formula (I)

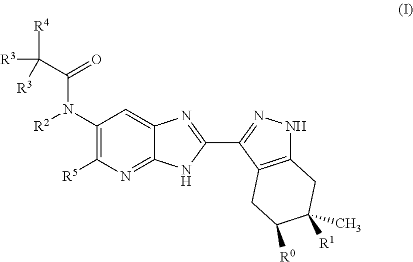

and pharmaceutically acceptable salts thereof, wherein R⁰ to R⁵ are as defined in the description; to their use in medicine; to compositions containing them; to processes for their preparation; and to intermediates used in such processes.

The benzimidazoles of Formula (I) are ITK inhibitors and are therefore potentially useful in the treatment of a wide range of disorders including, atopic dermatitis.

The invention relates to imidazopyridine derivatives, to their use in medicine, to compositions containing them, to processes for their preparation and to intermediates used in such processes. More especially the invention relates to inhibitors of interleukin-2-inducible T cell kinase (ITK) and their use in the treatment of diseases mediated by ITK, in particular skin diseases, such as dermatitis (e.g. atopic dermatitis).

Atopic dermatitis (AD) is a common chronic inflammatory skin disease with a prevalence in both children and adults. AD patients suffer from dry and pruritic skin lesions which can greatly affect their quality of life. Genetic and environmental factors can contribute to skin barrier disruption and immune hyper-activation which are key drivers of AD pathogenesis.

The pathogenic role for T cells and the Th2 cell-derived cytokines, IL-4 and IL-13, in AD has been shown through the clinical development of dupilumab, an antibody to the IL-4 receptor that blocks the activity of both IL-4 and IL-13. The important activity of these cytokines is also consistent with the early clinical efficacy that has been observed with Janus kinase (JAK) inhibitors, which block signaling of IL-4 and IL-13 as well as additional inflammatory cytokines produced in the skin. A therapeutic strategy that can effectively control the production of IL-4 and IL-13 is an alternative approach to modulate this pathway. Additionally, Th1 cells, Th22 cells, and Th17 cells and the cytokines which they produce, IFNγ, IL-22, and IL-17, respectively, also contribute to AD pathogenesis.

An effective anti-inflammatory for AD would modulate the predominant T cell driven inflammatory response. Interleukin-2-inducible T cell kinase (ITK) is a member of the Tec family of tyrosine kinases. ITK expression is largely limited to immune cells such as T, natural killer (NK), natural killer T (NKT), and mast cells. In T cells, ITK amplifies T cell receptor (TCR)-dependent signals to promote T cell activation, cytokine production, and T cell proliferation. ITK deletion or inhibition of ITK activity in T cells results in suppression of TCR-induced IL-4 and IL-13 production, which plays a central role in contributing to the pathophysiology of AD. An ITK inhibitor is expected to have additional efficacy compared to an antagonist of the IL-4 receptor, as ITK also contributes to TCR-dependent production of numerous pro-inflammatory cytokines such as IL-2, IL-17, IL-22, IL-31, IFNγ, and TNF-α. Additionally, ITK deficient CD8+ T cells demonstrate impaired cytotoxic T lymphocyte expansion, reduced degranulation and defective cytolytic capacity. ITK deficient mice and/or mice treated with an ITK inhibitor demonstrate reduced disease in models of type I diabetes, lymphoproliferative disease, allergy/asthma, and airway hyperresponsiveness. Moreover, ITK-deficient mice or mice treated with an ITK inhibitor demonstrate reduced skin inflammation in models of dermatitis. Elevated levels of ITK were described in peripheral T cells from patients with moderate to severe AD, and ITK expression is elevated in skin lesions from AD patients.

Additionally, tropomyosin receptor kinases (TRKs) are expressed by cells in the skin such as keratinocytes, neurons, mast cells, and basophils. Both TRKA and its ligand, nerve growth factor (NGF), are present in the skin and their expression is enhanced in AD skin lesions. Levels of NGF in skin lesions from AD patients have been demonstrated to correlate with itch severity. Cytokines IL-4 and IL-13 which contribute to AD pathogenesis have been demonstrated to enhance TRKA expression by keratinocytes. In addition to regulating development and maintenance of neurons, NGF can sensitize nociceptors and promote pruritis in the skin. Pruritis is a major factor contributing to reduced quality of life for AD patients. A therapy which can suppress pruritis would not only provide relief for patients, but may also break the itch-scratch cycle which contributes to the barrier disruption and thus reduce the course and chronicity of the disease.

NGF is also expressed by and has effects on non-neuronal cells. NGF induces keratinocyte proliferation, promotes basophil activation, stimulates mast cell degranulation, and contributes to neurogenic itch and inflammation. Furthermore, TRKA expression has been reported on TCR-stimulated peripheral blood T cells and T cells collected from synovial fluid from arthritis patients, and NGF induces proliferation of T cells. Thus, inhibiting TRKA in the skin may suppress dermal inflammation in addition to reducing pruritis.

These data suggest that an ITK inhibitor will suppress pathogenic T cell responses and reduce cytokine production, and therefore have therapeutic value in the treatment of a variety of inflammatory and autoimmune diseases, including dermatological conditions, such as atopic dermatitis, contact dermatitis, psoriasis, alopecia areata, and vitiligo. Moreover an inhibitor of both ITK and TRKA activity should be of particular advantage in the treatment of dermatological conditions, such as those just mentioned (e.g. atopic dermatitis).

### REFERENCES

Benecke H, et al. Expert Opin. Invest. Drugs. 2013; 22:1167-1179;
Bissonette R, Papp K A, et al. Brit. J. Derm. 2016; 175:902-911;
Botchkarev V A, Yaar M, Peters EMJ et al. J. Invest. Dermatology. 2006; 126:1719-1727;
Brunner P M, et al. J Allergy Clin Immunol. 2017; 139(4S):S65-S76;
Kapnick S M, Stinchcombe J C, et al. Immunol. 2017; 198:2699-2711;
Lin T A, McIntyre K W, et al. Biochemistry 2004; 43:11056-11062;
Matsumura S, Terao M, et al. J. Derm. Science 2015; 78:215-223;
Otsuka A, Nomura T, et al. Immunological Reviews. 2017; 278:246-262;
Raychaudhuri S P, Raychaudhuri S K, et al. Arthritis & Rheumatism 2011; 63:3243-3252;
Sabat R, Wolk K, et al. Seminars in Immunopathology 2019; 41:359-377;
Sahu N, and August A. Curr. Top. Med. Chem. 2009; 9:690-703;
Von Bonin A, Rausch A, et al. Exp. Derm. 2010; 20:41-47;
Yamaguchi J, Aihara M, et al. J. Dermatol. Science. 2008; 53:48-54

According to a first aspect of the invention there is provided a compound of Formula (I)

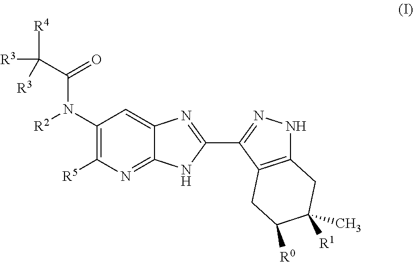

or a pharmaceutically acceptable salt thereof, wherein:
R⁰ is H and R¹ is methyl; or R⁰, R¹ and the carbon atoms to which they are attached form cyclopropyl, optionally gem-difluoro substituted;
R² is H, (C₁-C₄)alkyl, hydroxy(C₁-C₄)alkyl or (C₁-C₄)alkyl substituted by one, two or three F;
each R³ is independently H, F or (C₁-C₄)alkyl; or
both R³ taken together with the carbon atom to which they are attached form:
   (C₄-C₇)cycloalkyl;
   (C₄-C₇)cycloalkyl substituted by one, two or three F;
   (C₄-C₇)cycloalkyl substituted by one or two R⁶;
   (C₅-C₇)bicycloalkyl;
   (C₅-C₇)bicycloalkyl substituted by one or two F;
   C-linked 6-membered saturated heterocycloalkyl containing one N or one O, optionally substituted by oxo; or
   C-linked 6-membered saturated heterobicycloalkyl containing one N or one O, optionally substituted by oxo;
R⁴ is H; F; (C₁-C₆)alkyl, optionally substituted by one, two or three F; —NR⁷R⁸; (C₄-C₇)cycloalkyl; C-linked 6-membered saturated heterocycloalkyl containing one N or one O, wherein said heterocycloalkyl is optionally substituted by oxo; N-linked 6-, 7- or 8-membered saturated heterocycloalkyl containing one N and optionally one O, wherein said heterocyclalkyl is optionally substituted by oxo; N-linked 6-, 7- or 8-membered saturated heterobicycloalkyl containing one N and optionally one O, wherein said heterobicyloalkyl is optionally substituted by oxo; or N-linked 5- or 6-membered heteroaryl containing one or two N;
R⁵ is H; halogen; (C₁-C₆)alkyl; (C₁-C₆)alkoxy; or (C₁-C₆)alkoxy substituted by (C₁-C₄)alkoxy;
each R⁶ is independently (C₁-C₃)alkyl;
R⁷ is (C₁-C₆)alkyl; and
R⁸ is (C₁-C₆)alkyl or —C(O)(C₁-C₆)alkyl.

Described below are embodiments of this first aspect of the invention, where for convenience E1 is identical thereto.
E1 A compound of Formula (I) or a pharmaceutically acceptable salt thereof as defined above.
E2 A compound or a pharmaceutically acceptable salt thereof according to embodiment E1 wherein R^{a} is H and R¹ is methyl.
E3 A compound or a pharmaceutically acceptable salt thereof according to embodiment E1 of Formulae (Ia) or (Ib)

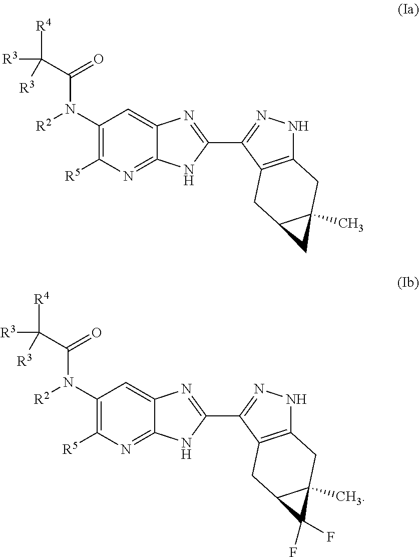

E4 A compound or a pharmaceutically acceptable salt thereof according to embodiment E3 of Formula (Ia).
E5 A compound or a pharmaceutically acceptable salt thereof according to embodiment E2 of Formula (Ib).
E6 A compound or a pharmaceutically acceptable salt according to any one of embodiments E1 to E5 thereof wherein R² is H, methyl, ethyl or hydroxyethyl.
E7 A compound or a pharmaceutically acceptable salt thereof according to embodiment E6 wherein R² is methyl.
E8 A compound or a pharmaceutically acceptable salt thereof according to any one of embodiments E1 to E7 wherein each R³ is independently H, F or (C₁-C₃)alkyl.
E9 A compound or a pharmaceutically acceptable salt thereof according to embodiment E8 wherein each R³ is H or each R³ is methyl.
E10 A compound or a pharmaceutically acceptable salt thereof according to embodiment E8 wherein each R³ is F.
E11 A compound or a pharmaceutically acceptable salt thereof according to embodiment E8 wherein one R³ is H and the other R³ is methyl.
E12 A compound or a pharmaceutically acceptable salt thereof according to embodiment E11 of Formula (Ic)

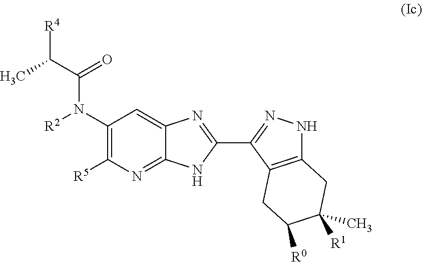

E13 A compound or a pharmaceutically acceptable salt thereof according to any one of embodiments E1 to E7 wherein both R³ taken together with the carbon atom to which they are attached form:
   (C₄-C₇)cycloalkyl;
   (C₄-C₇)cycloalkyl substituted by one, two or three F;
   (C₄-C₇)cycloalkyl substituted by one or two R⁶;
   (C₅-C₇)bicycloalkyl;
   (C₅-C₇)bicycloalkyl substituted by one or two F;
   C-linked 6-membered saturated heterocycloalkyl containing one N or one O, optionally substituted by oxo; or
   C-linked 6-membered saturated heterobicycloalkyl containing one N or one O, optionally substituted by oxo.
E14 A compound or a pharmaceutically acceptable salt thereof according to embodiment E13 wherein both R³ taken together with the carbon atom to which they are attached form (C₄-C₇)cycloalkyl; (C₄-C₇)cycloalkyl substituted by one, two or three F; or (C₄-C₇)cycloalkyl substituted by one or two R⁶.
E15 A compound or a pharmaceutically acceptable salt thereof according to embodiment E14 wherein both R³ taken together with the carbon atom to which they are attached form (C₄-C₆)cycloalkyl; (C₄-C₆)cycloalkyl substituted by one or two F; or (C₄-C₆)cycloalkyl substituted by one or two methyl.
E16 A compound or a pharmaceutically acceptable salt thereof according to embodiment E13 wherein both R³ taken together with the carbon atom to which they are attached form (C₅-C₇)bicycloalkyl or (C₅-C₇)bicycloalkyl substituted by one or two F.
E17 A compound or a pharmaceutically acceptable salt thereof according to embodiment E16 wherein both R³ taken together with the carbon atom to which they are attached form (C₅-C₇)bicycloalkyl substituted by one or two F.
E18 A compound or a pharmaceutically acceptable salt thereof according to embodiment E13 wherein both R³ taken together with the carbon atom to which they are attached form C-linked 6-membered saturated heterocycloalkyl containing one N or one O, optionally substituted by oxo; or C-linked 6-membered saturated heterobicycloalkyl containing one N or one O, optionally substituted by oxo.
E19 A compound or a pharmaceutically acceptable salt thereof according to embodiment E18 wherein both R³ taken together with the carbon atom to which they are attached form C-linked 6-membered saturated heterocycloalkyl containing one O.
E20 A compound or a pharmaceutically acceptable salt thereof according to embodiment E18 wherein both R³ taken together with the carbon atom to which they are attached form C-linked 6-membered saturated heterobicycloalkyl containing one O.
E21 A compound or a pharmaceutically acceptable salt thereof according to any one of embodiments E1 to E20 wherein R⁴ is H; F; (C₁-C₄)alkyl, optionally substituted by one or two F; —NR⁷R⁸; (C₄-C₇)cycloalkyl; C-linked 6 membered saturated heterocycloalkyl containing one O; N-linked 6-, 7- or 8-membered saturated heterocycloalkyl containing one N and optionally one O, wherein said heterocyclalkyl is optionally substituted by oxo; N-linked 6-, 7- or 8-membered saturated heterobicycloalkyl containing one N and optionally one O; or N-linked 5-membered heteroaryl containing two N.
E22 A compound or a pharmaceutically acceptable salt thereof according to embodiment E21 wherein R⁴ is H; F; (C₁-C₄)alkyl, optionally substituted by one or two F; or —NR⁷R⁸.
E23 A compound or a pharmaceutically acceptable salt thereof according to any one of embodiments E1 to E22 wherein R⁷ is (C₁-C₃)alkyl.
E24 A compound or a pharmaceutically acceptable salt thereof according to any one of embodiments E1 to E23 wherein R⁸ is —C(O)(C₁-C₃)alkyl.
E26 A compound or a pharmaceutically acceptable salt thereof according to embodiment E22 wherein R⁴ is H or F.
E27 A compound or a pharmaceutically acceptable salt thereof according to embodiment E26 wherein R⁴ is H.
E28 A compound or a pharmaceutically acceptable salt thereof according to embodiment E21 wherein R⁴ is (C₄-C₇)cycloalkyl; C-linked 6 membered saturated heterocycloalkyl containing one O; N-linked 6-, 7- or 8-membered saturated heterocycloalkyl containing one N and optionally one O, wherein said heterocyclalkyl is optionally substituted by oxo; N-linked 6-, 7- or 8-membered saturated heterobicycloalkyl containing one N and optionally one O; or N-linked 5-membered heteroaryl containing two N.
E29 A compound or a pharmaceutically acceptable salt thereof according to embodiment E28 wherein R⁴ is (C₄-C₆)cycloalkyl; C-linked 6-membered saturated heterocycloalkyl containing one O; N-linked 6- or 7-membered saturated heterocycloalkyl containing one N and optionally one O, wherein said heterocyclalkyl is optionally substituted by oxo; N-linked 7- or 8-membered saturated heterobicycloalkyl containing one N and one O; or N-linked 5-membered heteroaryl containing two N.
E30 A compound or a pharmaceutically acceptable salt thereof according to embodiment E29 wherein R⁴ is (C₄-C₆)cycloalkyl; C-linked 6-membered saturated heterocycloalkyl containing one O; N-linked 6-membered saturated heterocycloalkyl containing one N and O, wherein said heterocyclalkyl is optionally substituted by oxo; N-linked 7- or 8-membered saturated heterobicycloalkyl containing one N and one O; or N-linked pyrazole.
E31 A compound or a pharmaceutically acceptable salt thereof according to embodiment E30 wherein R⁴ is C-linked 6-membered saturated heterocycloalkyl containing one 0.
E32 A compound or a pharmaceutically acceptable salt thereof according to embodiment E31 wherein R⁴ is tetrahydro-2H-pyran-4-yl.
E33 A compound or a pharmaceutically acceptable salt thereof according to embodiment E30 wherein R⁴ is N-linked 6-membered saturated heterocycloalkyl containing one N and O, wherein said heterocyclalkyl is optionally substituted by oxo.
E34 A compound or a pharmaceutically acceptable salt thereof according to embodiment E33 wherein R⁴ is N-linked morpholinyl.
E35 A compound or a pharmaceutically acceptable salt thereof according to embodiment E30 wherein R⁴ is N-linked 7- or 8-membered saturated heterobicycloalkyl containing one N and one O.
E36 A compound or a pharmaceutically acceptable salt thereof according to embodiment E35 wherein R⁴ is

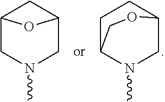

E37 A compound or a pharmaceutically acceptable salt thereof according to embodiment E30 wherein R⁴ is N-linked pyrazole.
E38 A compound or a pharmaceutically acceptable salt thereof according to any one of embodiments E1 to E37 wherein R⁵ is H, F, (C₁-C₃)alkyl, (C₁-C₃)alkoxy, or (C₁-C₃)alkoxy substituted by (C₁-C₃)alkoxy.
E39 A compound or a pharmaceutically acceptable salt thereof according to embodiment E38 wherein R⁵ is H, F, methyl, —OCH₃ or —OC₂H₅OCH₃.
E40 A compound or a pharmaceutically acceptable salt thereof according to embodiment E39 wherein R⁵ is H.
E41 A compound or a pharmaceutically acceptable salt thereof according to embodiment E1 selected from any one of Examples 1 to 120.
E42 A compound or a pharmaceutically acceptable salt thereof according to embodiment E1 selected from:
(R)-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide; and
2,2-difluoro-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)acetamide.

In compounds of Formula (I):
Alkyl means a straight or branched chain hydrocarbon group of formula —CₙH₍₂ₙ₊₁₎. Examples of alkyl include methyl, ethyl, n-propyl, i-propyl, n-butyl, i-butyl, sec-butyl and t-butyl.
Alkyloxy means an alkyl substituent attached through an oxygen atom. Examples of alkyloxy include methoxy, ethoxy, n-propoxy, i-propoxy, n-butoxy, i-butoxy, sec-butoxy and t-butoxy.
Cycloalkyl means a cyclic hydrocarbon group of formula —CₙH₍₂ₙ₋₁₎ containing at least three carbon atoms. Examples of cycloalkyl include cyclopropyl, cyclobutyl, cyclopentyl and cyclohexyl.
Bicycloalkyl means a bicyclic hydrocarbon group in which the two rings are fused, spiro-fused or form a bridged structure. Examples of (C₅-C₇)bicycloalkyl include:

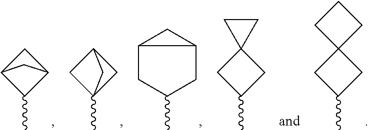

Examples of C-linked 6-membered saturated heterocycloalkyl containing one N or one O include tetrahydro-2H-pyran-2-yl, tetrahydro-2H-pyran-3-yl and tetrahydro-2H-pyran-4-yl.
Examples of C-linked 6-membered saturated heterobicycloalkyl containing one N or one O include

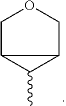

Examples of N-linked 6-, 7- or 8-membered saturated heterocycloalkyl containing one N and optionally one O include piperidinyl, morpholinyl, 1,3-oxazinan-3-yl and 1,4-oxazepan-4-yl.
Oxo refers to a double bonded oxygen (═O). Preferably an oxo substituent in an N-linked heterocycloalkyl or heterobicycloalkyl containing one N and optionally one O is alpha to the N atom, e.g. as follows:

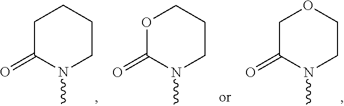

Examples of; N-linked 6-, 7- or 8-membered saturated heterobicycloalkyl containing one N and optionally one O include

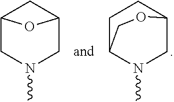

Examples of halogen include fluoro (F), chloro (Cl), bromo (Br) and iodo (I). Hereinafter, all references to compounds of the invention include compounds of Formula (I) or pharmaceutically acceptable salts, solvates, or multi-component complexes thereof, or pharmaceutically acceptable solvates or multi-component complexes of pharmaceutically acceptable salts of compounds of Formula (I), as discussed in more detail below.

Preferred compounds of the invention are compounds of Formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, 1,5-naphathalenedisulfonate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Hemisalts of acids may also be formed, for example, hemisulphate and hemitartrate salts.

The skilled person will appreciate that the aforementioned salts include ones wherein the counterion is optically active, for example d-lactate, or racemic, for example dl-tartrate.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of Formula (I) may be prepared by one or more of three methods:
(i) by reacting the compound of Formula (I) with the desired acid;
(ii) by removing an acid-labile protecting group from a suitable precursor of the compound of Formula (I) using the desired acid; or
(iii) by converting one salt of the compound of Formula (I) to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of Formula (I) or pharmaceutically acceptable salts thereof may exist in both unsolvated and solvated forms. The term ‘solvate’ is used herein to describe a molecular complex comprising a compound of Formula (I) or a pharmaceutically acceptable salt thereof and one or more pharmaceutically acceptable solvent molecules, for example, ethanol. The term ‘hydrate’ is employed when said solvent is water. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) of compounds of Formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The compounds of the invention may be administered as prodrugs. Thus certain derivatives of compounds of Formula (I) which may have little or no pharmacological activity themselves can, when administered into or onto the body, be converted into compounds of Formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of Formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of Formula (I), that is, compounds formed in vivo upon administration of the drug. Examples of metabolites in accordance with the invention include:
(i) where the compound of Formula (I) contains an alkoxy group, a hydroxy derivative thereof (—(C₁-C₆)alkoxy→—OH); and
(ii) where the compound of Formula (I) contains a phenyl moiety, a phenol derivative thereof (-Ph→-PhOH).

Formula (I) contains an asymmetric cyclopropaindazolyl moiety and is stereospecifically defined (as the ‘4aS,5aR’ stereoisomer).

The skilled person will appreciate that one or more substituents in Formula (I) may introduce one or more additional asymmetric centres. For example, an additional asymmetric centre is present in compounds of Formula (I) wherein each R³ is different, and R⁴ is not the same as R³. Such an asymmetric centre is found in Example 1, a compound of Formulae (I) and (Ic). The skilled person will further appreciate that the stereoisomer of Example 1 may be depicted in different, but chemically identical, ways, for example as shown below, where the additional asymmetric centre is marked by an asterisk (*).

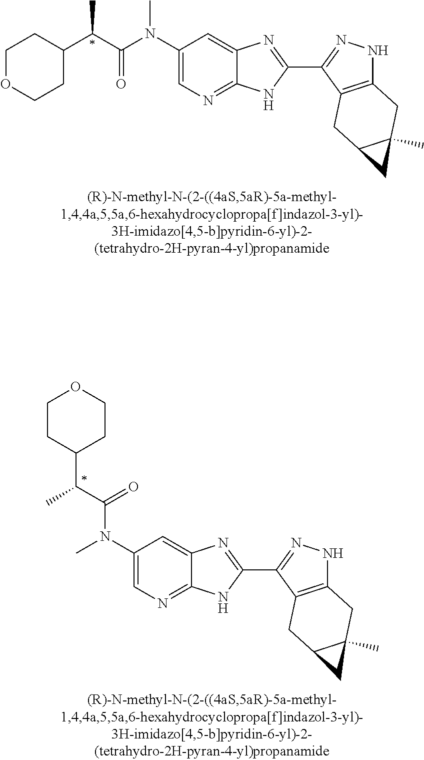

Compounds of the invention containing said one or more additional asymmetric centres can exist as two or more stereoisomers; included within the scope of the invention are all such stereoisomers (including epimers) of the compounds of the invention and mixtures of two or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Chiral chromatography using sub-and supercritical fluids may be employed. Methods for chiral chromatography useful in some embodiments of the present invention are known; see, for example, Smith, Roger M., Loughborough University, Loughborough, UK; Chromatographic Science Series (1998), 75 (Supercritical Fluid Chromatography with Packed Columns), pp. 223-249 and references cited therein.

Mixtures of stereoisomers may be separated by conventional techniques known to those skilled in the art; see, for example, “Stereochemistry of Organic Compounds” by E. L. Eliel and S. H. Wilen (Wiley, New York, 1994.

Where structural isomers are interconvertible via a low energy barrier, tautomeric isomerism (‘tautomerism’) and conformational isomerism can occur.

Tautomerism can take the form of proton tautomerism in compounds of Formula (I), as illustrated below in Formula (I) generally, and Example 1 specifically, with respect to the imidazopyridine group:

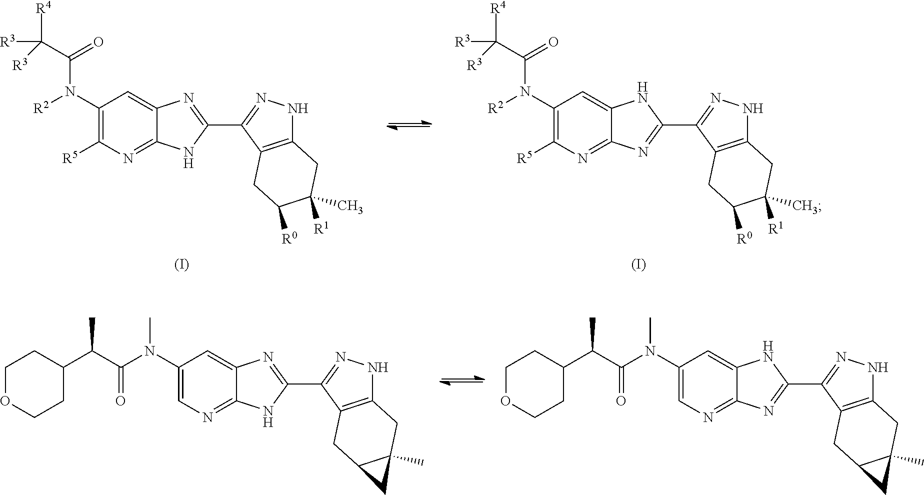

The skilled person will appreciate that proton tautomerism can also take place on the pyrazole ring in compounds of Formula (I).

While, for conciseness, the compounds of Formula (I) have been drawn herein in a single tautomeric form, all possible tautomeric forms, and mixtures thereof, are included within the scope of the invention.

Conformational isomerism is a form of stereoisomerism in which the isomers of a compound can be interconverted exclusively by rotations about single bonds. Such isomers are generally referred to as conformational isomers or conformers and, specifically, as rotamers. A “rotameric mixture”, or “mixture of rotamers”, describes a compound existing as a mixture of more than one of the possible conformational isomers. While, for conciseness, the compounds of Formula (I) have been drawn in a single conformational form, all possible conformers, and mixtures thereof, are included within the scope of the invention.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of: hydrogen, such as ²H and ³H; carbon, such as ¹¹C, ¹³C and ¹⁴C; fluorine, such as ¹⁸F; chlorine, such as ³⁶Cl; iodine, such as ¹²³I and ¹²⁵I; nitrogen, such as ¹³N and ¹⁵N; oxygen, such as ¹⁵O, ¹⁷O and ¹⁸O.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium (D), i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁵O and ¹³N, can be useful in Positron Emission Topography (PET) studies for examining substrate receptor occupancy.

Isotopically-labeled compounds of Formula (I) can generally be prepared by conventional techniques known to those skilled in the art or by processes analogous to those described in the accompanying examples and preparations using an appropriate isotopically-labeled reagent in place of the non-labeled reagent previously employed.

Also, within the scope of the invention are intermediate compounds as hereinafter defined, all salts, solvates and complexes thereof, and all solvates and complexes of salts thereof as defined hereinbefore for compounds of Formula (I). The invention includes all polymorphs of the aforementioned species and crystal habits thereof.

When preparing a compound of Formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

The compounds of the invention may be prepared by any method known in the art for the preparation of compounds of analogous structure. In particular, the compounds of the invention can be prepared by the procedures described by reference to the schemes that follow, or by the specific methods described in the examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of Formula (I). It will be further appreciated that it may be necessary or desirable to carry out the transformations in a different order from that described in the schemes, or to modify one or more of the transformations, to provide the desired compound of the invention.

Compounds of the present invention contain two or more stereogenic centers, with the stereochemical designation (R) or (S). The skilled person will appreciate that all the synthetic transformations can be conducted on either enantioenriched or racemic compounds, and that the resolution to the desired stereoisomer may take place at any point in the synthesis, using well known methods described herein and/or known in the art.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect hydroxyl, carboxyl and/or amino groups. The protecting groups used in the preparation of the compounds of the invention may be used in conventional manner; see, for example, those described in ‘Greene's Protective Groups in Organic Synthesis’ by Theodora W Greene and Peter G M Wuts, fifth edition, (John Wiley and Sons, 2014), incorporated herein by reference, and in particular chapters 2, 5 and 7 respectively, which also describes methods for the removal of such groups.

In the following general processes and unless otherwise stated:
R¹ to R⁶ are as previously defined for a compound of Formula (I);
R is alkyl, such as methyl or ethyl, or in the case of Formulae 3 and 4, two R may be taken together with the oxygen atoms to which they are attached to form a cyclic acetal;
PG is a suitable amino protecting group, such as a silyl ether (e.g. SEM), an alkoxy carbonyl (e.g. BOC), acetyl (Ac), benzyl (e.g. PMB) or dihydropyran (DHP) protecting group; and
X is F or Cl.

A substituted pyrazole of Formula 11 may be prepared as shown in Scheme 1.

Compound 1 (3-methoxytoluene) can be reduced to the corresponding 1,4-diene Compound 2 by a Birch reduction (Mander, L. N. Comprehensive Organic Synthesis; Trost, B. M. and Fleming, I., Ed.; Pergamon: Oxford, 1991, Vol. 8, pp. 489-521), using an alkali metal such as Li or Na in liquid ammonia at temperatures below −30° C.

Preparation of an olefinic acetal of Formula 3 from the 1,4-diene Compound 2 can proceed under catalytic acid conditions, e.g. using pTSA or CSA in the presence of alkyl primary alcohols such as methanol or ethanol, or a diol such as ethylene glycol, with or without a solvent such as DCM or other aprotic solvent, at a temperature between 0-100° C., such as 0-25° C.

Conversion of an olefin of Formula 3 into a cyclopropane of Formula 4 may proceed via dihalocarbene addition or Simmons-Smith cyclopropanation (Charette, A. B.; Beauchemin, A. Simmons-Smith Cyclopropanation Reaction. Org. React. 2001, 58, p 1-415).

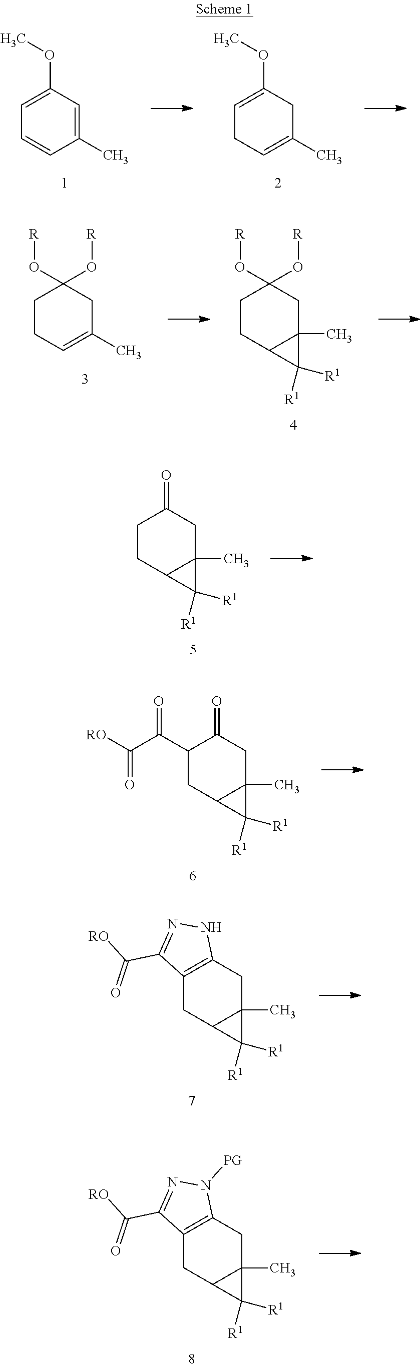

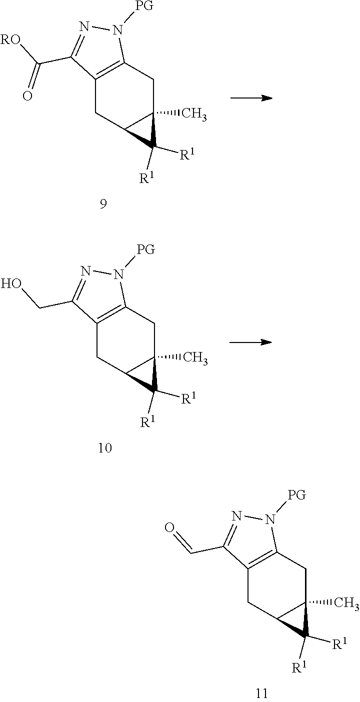

Deprotection of an acetal of Formula 4 to give a ketone of Formula 5 may be performed under acidic conditions, e.g. using HCl, H₂SO₄ or an organic acid such as pTSA, in a mixture of water and solvent such as THF.

Preparation of a diketone of Formula 6 can be achieved by reacting a ketone of Formula with: i) a dialkyl oxalate and 1-3 equivalents of a strong base, such as LDA, LiHMDS or KOtBu, in a polar aprotic solvent such as THF, at −78° C. to 25° C.; or ii) with an alkoxide in a corresponding alcoholic solvent (e.g. sodium ethoxide in ethanol) at temperatures between 0° C. and reflux.

Condensation of a diketone of Formula 6 with hydrazine or hydrazine hydrate, in a protic solvent such as methanol or ethanol, at 25° C. to reflux, can provide a pyrazole of Formula 7. A hydrazine salt, such as the HCl salt, may also be used together with a corresponding molar equivalent of inorganic (e.g. K₂CO₃) or organic (e.g. Et₃N or iPr₂NEt) base.

Protection of a pyrazole of Formula 7 can be performed with SEM-Cl, DHP or another suitable protecting group to deliver a pyrazole of Formula 8, resolution of which to deliver the corresponding enantiomer of Formula 9 can be performed by supercritical fluid chromatography with the use of a chiral solid phase.

Reduction of an ester of Formula 9 to an alcohol of Formula 10 may be performed using LAH, in an aprotic solvent such as THF, at temperatures between 0° C. and reflux.

Oxidation of an alcohol of Formula 10 to an aldehyde of Formula 11 can be effected by: i) using an agent, such as PCC, PDC, or MnO₂, in an aprotic solvent; or ii) by catalysis, for example by using TEMPO/bleach and TPAP/NMO (Caron, S., Dugger, R. W., Gut Ruggeri, S., Ragan, J. A., Brown Ripin, D. H., Chem. Rev. 2006, 106, 2943-2989) or Swern oxidation conditions.

A substituted pyrazole of Formula 17 maybe prepared as shown in Scheme 2.

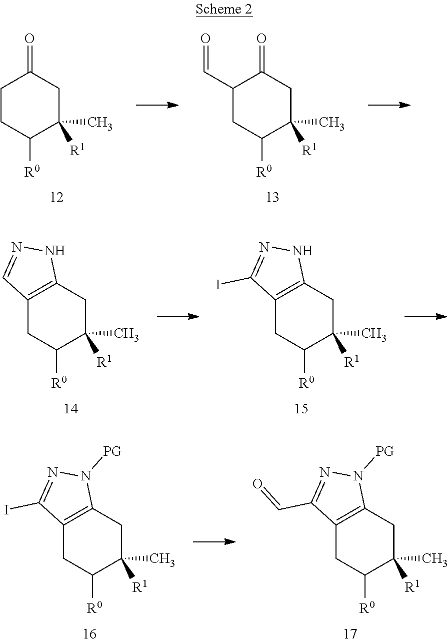

Treatment of a ketone of Formula 12 with an alkoxide base such as sodium ethoxide or KOtBu, or other bases such as LDA or LiHMDS, and a formylating agent such as ethyl formate in an aprotic solvent such as THF between −78° C. and 80° C. can provide a dicarbonyl of Formula 13.

Condensation of a dicarbonyl of Formula 13 with hydrazine or hydrazine hydrate, in a protic solvent such as methanol or ethanol, at 25° C. to reflux, can provide a pyrazole of Formula 14. A hydrazine salt, such as the HCl salt, may also be used together with a corresponding molar equivalent of inorganic (e.g. K₂CO₃) or organic (e.g. Et₃N or iPr₂NEt) base.

A pyrazole of formula 14 may be iodinated to provide an iodo-pyrazole of Formula 15 by treatment with iodine in a polar aprotic solvent such as DMF with addition of an alkali hydroxide such as KOH at a temperature between 0° C. and 50° C.

Protection of a pyrazole of Formula 15 can be performed with SEM-Cl, DHP or another suitable protecting group to deliver a pyrazole of Formula 16.

An iodo-pyrazole of Formula 16 may be converted to a formyl pyrazole of Formula 17 by treatment with a palladium-ligand complex such as Pd(dppf)Cl₂ in the presence of a base such as Et₃N and a hydride source such as triethylsilane in a polar aprotic solvent such as DMF under a pressurized atmosphere of CO (from 2-5 atm) and at a temperature between 30° C. and 100° C.

A compound of Formula (I) may be prepared as shown in Scheme 3.

A nitro aniline of Formula 19 be prepared via substitution of X in a compound of Formula 18 by nucleophilic aromatic substitution (S_{N}Ar) reaction with benzyl amine; at 25 to 100° C.; in the presence of a base, such as an inorganic base (e.g. sodium-, potassium-, or cesium carbonate, bicarbonate, hydroxide, or acetate), or an organic amine base such as Et₃N; in a polar aprotic solvent such as THF, DMF, DMAC, DMSO or NMP, or a protic solvent such as water, MeOH, EtOH or isopropanol, or a mixture thereof.

An ester of Formula 19 may be hydrolyzed using aqueous lithium, sodium or potassium hydroxide in a solvent such as MeOH, EtOH or THF, or mixture thereof, at a temperature between 20 C and reflux to provide an acid of Formula 20.

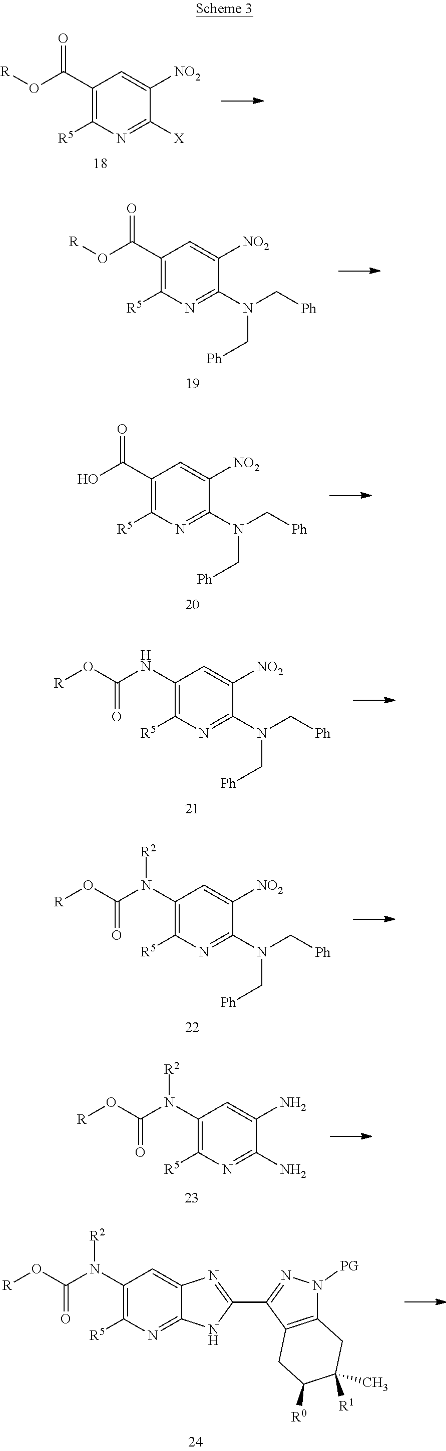

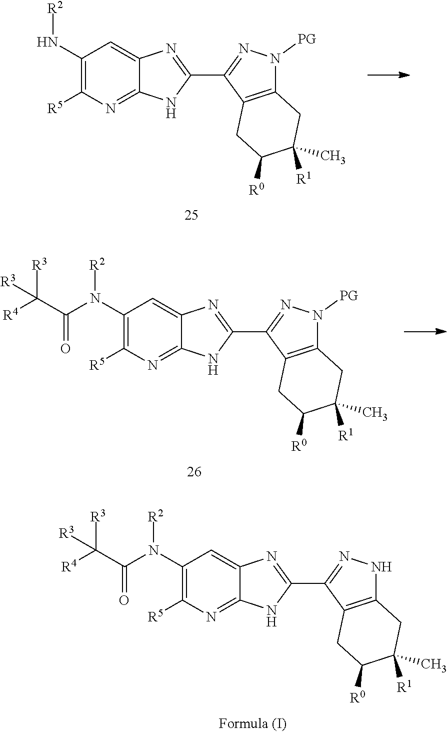

Preparation of a carbamate of Formula 21 may proceed from an acid of Formula 20 by treatment with diphenyl phosphoryl azide, in a solvent such as toluene, in the presence of a base such as Et₃N, and an alcohol such as tert-butyl alcohol or alternative alcohols such as methanol, ethanol and benzyl alcohol, at a temperature between 60° C. and 120° C.

Alkylation of a cabamate of Formula 21 to provide a carbamate of Formula 22 may be effected with an alkylating agent such as an alkyl halide or tosylate, in the presence of a base such as KOtBu or LiHMDS, in a polar aprotic solvent such as DMF or THF.

Reduction of a nitro aniline of Formula 22 (with concomitant deprotection) can be performed under hydrogenation conditions with Pd catalyst, such as 10% Pd/C or Pd(OH)₂/C under 1-5 atm H₂, in an alcoholic solvent such as methanol or ethanol, at a temperature between 20 and 100° C., to deliver a diamine of Formula 23.

A diamine of Formula 23 can be condensed with an aldehyde of Formulae 11 or 17 in a polar solvent, such as DMF with or 0-5 eq DMSO, and with an oxidant such as sodium metabisulfite, at a temperature between 90 and 150° C., to deliver an imidazopyridine of Formula 24. Alternatively, the condensation of compounds of Formula 23 with compounds of Formulae 11 or 17 can proceed in the presence aqueous sodium bisulfite, and ethanol or other alcoholic solvent, at 60° C. to reflux.

Selective deprotection of a protected amine of Formula 24 to deliver an amine of Formula can be achieved by reaction with ZnBr₂ (where R=t-butyl), in a non-polar solvent such as DCM.

An amine of Formula 25 may be acylated to provide an amide of Formula 26 with a carboxylic acid using standard amide coupling reagents such as EDCl, HATU, HBTU, BTFFH or T₃P; or by reaction with an alternate acylating agent, such an acid chloride, acid anhydride or acyl imidazole, in a solvent such as DCM or DMF, in the presence of an organic base such as Et₃N, at a temperature between 0° C. and reflux.

Removal of the protecting group in a compound of Formula 26 to deliver the corresponding compound of Formula (I) may be performed under conditions well known to the skilled person. For instance, when PG=SEM, the protecting group may be removed by use of TFA in DCM, optionally with added triethylsilane

The skilled person will appreciate that a compound of Formula 23 (and subsequently compounds of Formulae 24 to 26, and (1)) wherein R² is H may be prepared according to Scheme 3 directly from a compound of Formula 21.

An amide of Formula 26 may be prepared as shown in Scheme 4.

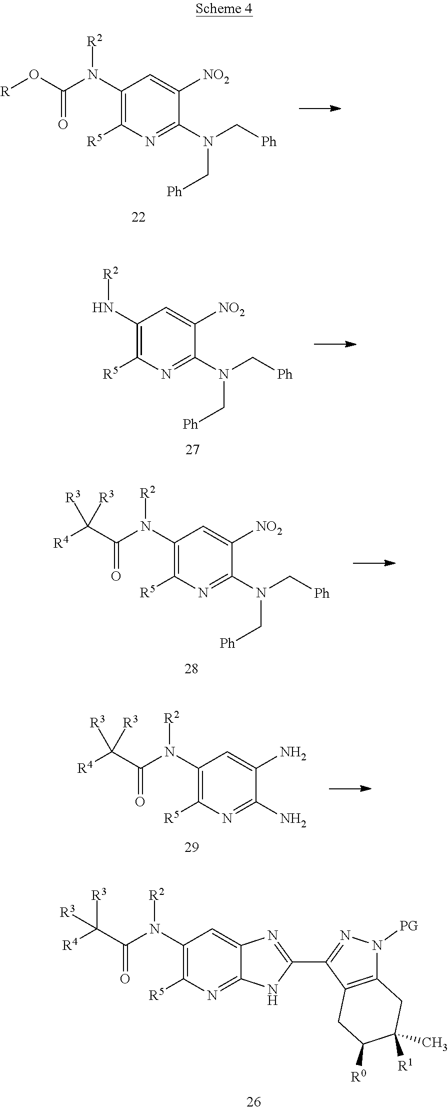

A protected amine of Formula 22 may be deprotected under conditions well known to the skilled person to provide an amine of Formula 27. For instance, when R=t-butyl, the carbamate may be removed by use of TFA in DCM or HCl (for instance 4 M HCl in 1,4 dioxane).

An amine of Formula 27 may be acylated to provide an amide of Formula 28 as described in Scheme 3 for the preparation of an amide of formula 26.

Reduction of a nitro aniline of Formula 28 (with concomitant deprotection, as required) can be performed under hydrogenation conditions described in Scheme 3 for the preparation of a diamine of formula 23 to deliver a diamine of Formula 29.

A diamine of Formula 29 can be condensed with an aldehyde of Formulae 11 or 17 to deliver a compound of formula 26 as described in Scheme 3 for the preparation of a compound of Formula 24.

A compound of Formula 35 may be prepared as described in Scheme 5.

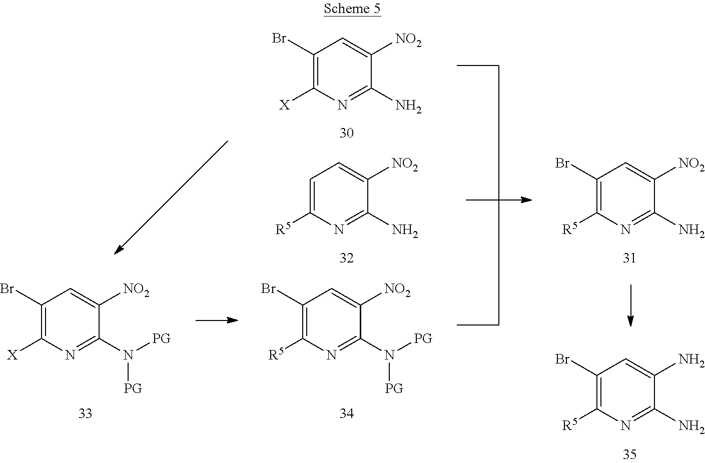

A nitro-aniline of Formula 31 may be prepared by substitution of X in a nitro-aniline of Formula 30 by a source of R⁵ under the S_{N}Ar conditions described above in Scheme 3 for the preparation of a nitro-aniline of Formula 19. Alternatively, a nitro aniline of Formula 32 may brominated with an electrophilic brominating reagent such as NBS or Br₂ under conditions well known to the skilled person following the rules of electrophilic aromatic substitution to provide a nitro-aniline of Formula 31 wherein R⁵ is Br.

A nitro-aniline of Formula 30 may be protected with a protecting group, such as BOC, to provide a compound of Formula 33. A protected nitro aniline of Formula 34 may be prepared by substitution of X in a compound of Formula 33 under conditions described just above for the preparation of a compound of Formula 31. Removal of the protecting groups in a compound of Formula 34 to provide a nitro-aniline of Formula 31 can be accomplished by the skilled person using standard methods.

A compound of Formula 35 may be prepared by reduction of a nitro-aniline of Formula 31 by use of a metal such as Zn or Fe in AcOH as solvent or mixture of organic solvent such as THF with aqueous ammonium chloride, at a temperature between 20-100° C.

A compound of Formula 38 may be prepared as described in Scheme 6.

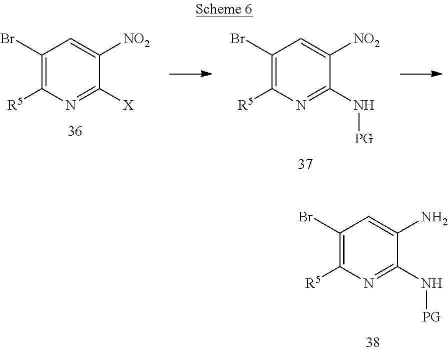

A compound of Formula 36 may be substituted by a nitrogen nucleophile such as benzyl amine or 4-methoxybenzyl amine to provide a nitro-aniline of Formula 37, which may be reduced as described previously in Scheme 5, for the preparation of a diamine of formula 35, to provide a diamine of Formula 38.

A compound of Formula 40 may be prepared as described in Scheme 7.

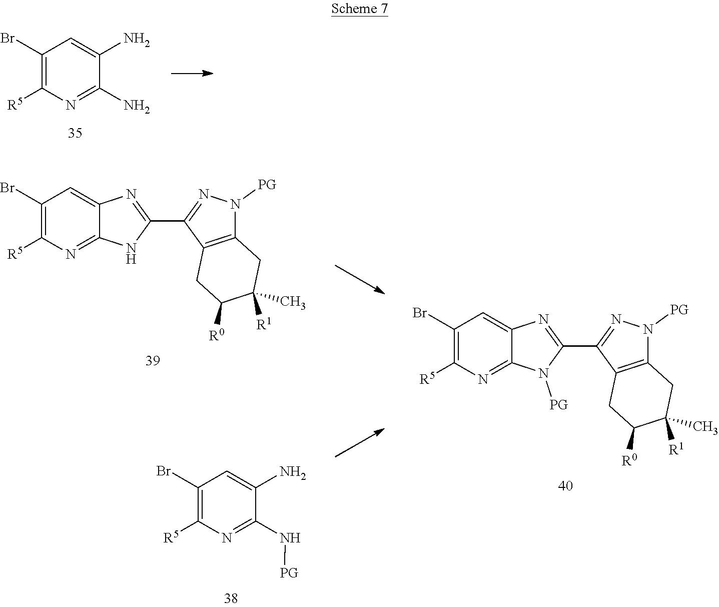

A diamine of Formula 35 can be condensed with an aldehyde of Formulae 11 or 17 in a polar solvent, such as DMF with or 0-5 eq DMSO, and with an oxidant such as Na₂S₂O₅, at a temperature between 90 and 150° C., to deliver an imidazopyridine of Formula 39. Protection of an imidazopyridine of Formula 39 can be performed with SEM-Cl, DHP or another suitable protecting group, to deliver a compound of Formula 40 which may exist as a mixture of regioisomers.

Additionally, a diamine of Formula 38 may be condensed with an aldehyde of Formulae 11 or 17 as described above for the preparation of an imidazopyridine of Formula 39, to provide an imidazopyridine of Formula 40.

A compound of Formula (I) may also be prepared as described in Scheme 8.

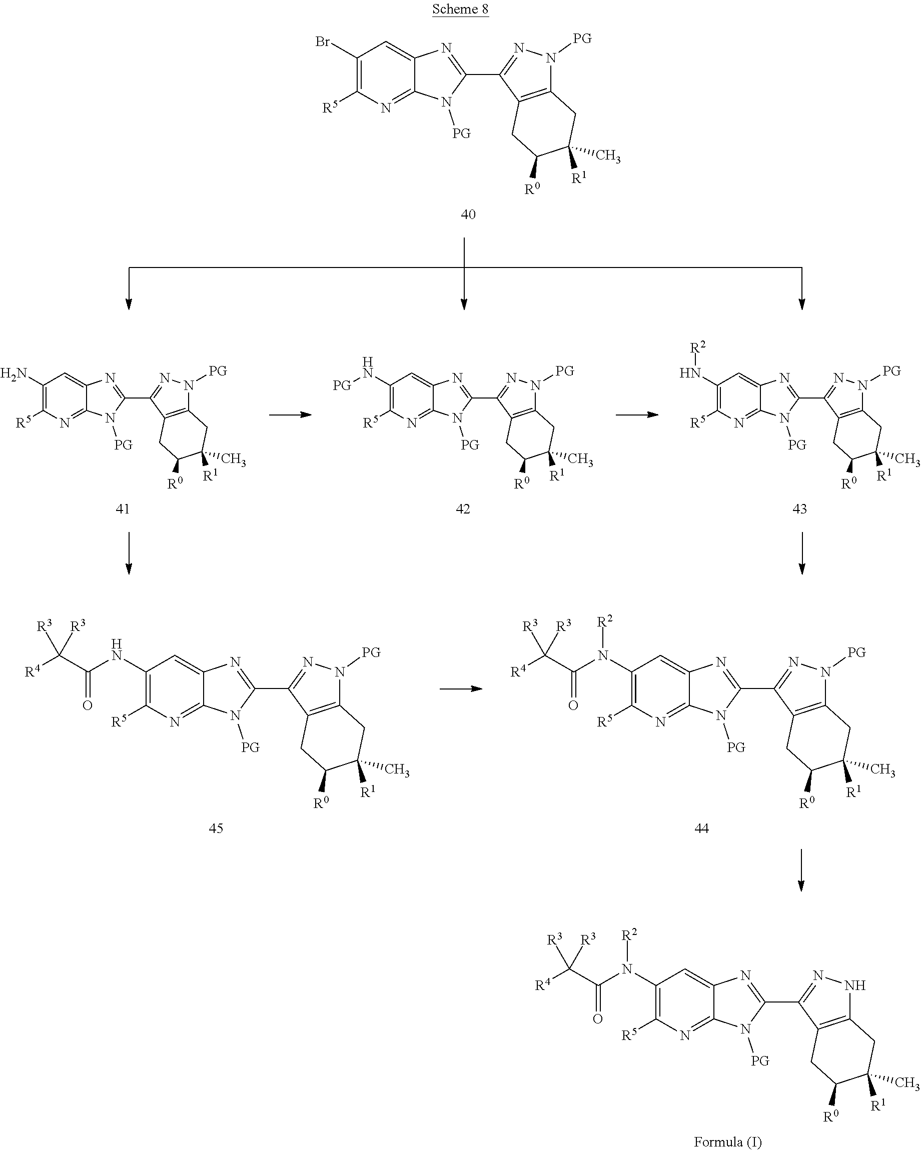

A compound of Formula 40 may be converted directly to an amine of Formulae 41, 42 or 43 via a transition metal catalyzed cross coupling reaction such as Buchwald-Hartwig coupling (Bernhardson, D. J., Widlicka, D. W., Singer, R. A., Cu-Catalyzed Couplings of Heteroaryl Primary Amines and (Hetero)aryl Bromides with 6-Hydroxypicolinamide Ligands, Org. Process Res. Dev. 2019, 23, 1538-1551).

A compound of Formula 41 may be protected (for example PG=BOC or CBZ) to give a compound of Formula 42 under conventional conditions.

An amine of Formula 43 may be prepared by alkylation of a compound of Formula 42 as described in Scheme 3 for the preparation of a compound of Formula 22, followed by deprotection as described in Scheme 3 for the preparation of an amine of Formula 25.

An amine of Formula 43 may be acylated to give an amide of Formula 44 which may be subsequently deprotected to provide a compound of Formula (I), as described in Scheme 3 for the preparation of an amide of Formula 26 and its deprotection to a compound of Formula (I).

The skilled person will appreciate that a compound of Formula 44 (and subsequently a compound of Formulae (I)) wherein R² is H may be prepared according to Scheme 8 directly from a compound of Formula 42, and that likewise a compound of Formula (I) wherein R² is H may be prepared directly from a compound of Formula 45.

The skilled person will appreciate that an amine of Formula 41 may be converted into an amide of Formula 44 via an amide of Formula 45 by reversing the alkylation and acylation steps, i.e. by first acylating and then alkylating.

Compounds of Formulae 1, 12, 18, 30, 32, 35 and 36 may be acquired from commercial sources, prepared by analogy with literature methods, or obtained by the methods described in the Experimental section that follows or variations of the same, well known to the skilled person.

Compounds of the invention intended for pharmaceutical use may be administered in amorphous or crystalline form or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

Compounds of the invention may be administered by any suitable route in the form of a pharmaceutical composition adapted to such a route, and in a dose effective for the treatment intended. Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

Modes of administration for compounds of the invention include oral, parenteral, topical, rectal, vaginal, ocular and aural administration.

Oral administration may involve swallowing, so that a compound of the invention enters the gastrointestinal tract, or buccal or sublingual administration, such that the compound enters the bloodstream directly from the mouth.

Parenteral administration may involve injecting a compound of the invention into the bloodstream, muscle or an internal organ, where the injection may be intravenous, intraarterial, intraperitoneal, intrathecal, intraventricular, intraurethral, intrasternal, intracranial, intramuscular or subcutaneous. Parenteral administration may employ needle (including microneedle) injectors, needle-free injectors and infusion techniques.

Topical administration is preferred and includes:
administration to the skin, nail, hair, claw, hoof, mucosa;
dermal or transdermal administration;
intranasal administration or administration by inhalation;
rectal or vaginal administration; and
administration directly to the eye or ear.

The term “transdermal administration” refers to the diffusion of a compound of the invention across the barrier of the skin, nail, hair, claw or hoof resulting from topical administration or other application of a composition. Transdermal delivery includes delivery through any portion of the skin, nail, hair, claw or hoof and absorption or permeation through the remaining portion.

Topical administration of a compound of the invention can result in distribution of the compound limited to the skin and surrounding tissues or, when the compound is removed from the treatment area by the bloodstream, can result in systemic exposure of the compound of the invention. Preferably, topical administration of a compound of the invention results in distribution of the compound limited to the skin and surrounding tissues. Where systemic exposure of the compound of the invention occurs, preferably the compound is rapidly metabolized so that systemic exposure of compound of the invention is minimized. Minimizing systemic exposure can reduce unwanted biological effects (i.e. side effects).

In another aspect the invention provides a pharmaceutical composition comprising a compound of the invention and a pharmaceutically acceptable excipient.

Pharmaceutical compositions suitable for the delivery of compounds of the invention and methods for their preparation will be readily apparent to those skilled in the art. Such compositions and preparative methods may be found in, for example, “Remington's Pharmaceutical Sciences”, 19th Edition (Mack Publishing Company, 1995).

Pharmaceutical compositions are typically prepared by mixing a compound of the invention and one or more excipients. Excipients include materials such as carbohydrates, waxes, water soluble and/or swellable polymers, hydrophilic or hydrophobic materials, gelatin, oils, solvents, water, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and the like. Solvents may include water, ethanol, propylene glycol, polyethylene glycols (e.g., PEG400, PEG300), and mixtures thereof. The excipient(s) are chosen to facilitate manufacture, or use, of the pharmaceutical composition.

Pharmaceutical compositions may be prepared by conventional dissolution and mixing. For example, the compound of the invention may be dissolved in a solvent in the presence of one or more of the excipients described above. The dissolution rate of poorly water-soluble compounds may be enhanced by the use of a spray-dried dispersion, such as those described by Takeuchi, H., et al. in “Enhancement of the dissolution rate of a poorly water-soluble drug (tolbutamide) by a spray-drying solvent deposition method and disintegrants” J. Pharm. Pharmacol., 39, 769-773 (1987); and US2002/009494; incorporated herein by reference.

Solid dosage forms for oral administration of compounds of the invention include, for example, tablets, hard or soft capsules, lozenges, granules or powders, each containing at least one compound of the invention. In such solid dosage forms the compound of the invention is ordinarily combined with one or more pharmaceutically acceptable excipients. Solid dosage forms for oral administration such as tablets and capsules may be prepared with enteric coatings.

Liquid dosage forms for oral administration of compounds of the invention include, for example, pharmaceutically acceptable emulsions, solutions, suspensions, syrups, and elixirs containing inert diluents commonly used in the art (e.g. water). Such compositions also may comprise excipients, such as wetting, emulsifying, suspending, flavoring (e.g. sweetening), and/or perfuming agents.

Parenteral formulations of compounds of the invention are typically aqueous solutions which may contain excipients such as salts, carbohydrates and buffers (preferably buffering to a pH of from 3 to 9). Formulations for parenteral administration may also be sterile non-aqueous solutions, or dried (e.g. lyophilised) forms to be administered on reconstitution with a suitable vehicle such as sterile, pyrogen-free water.

Pharmaceutical compositions for topical or transdermal administration of a compound of the invention include ointments, pastes, creams, lotions, gels, suppositories, powders, solutions, sprays, drops, inhalants and patches. The compound of the invention is admixed under sterile conditions with a pharmaceutically acceptable topical carrier and any preservatives or buffers as may be required. Compounds that are volatile may require admixture with formulating agents or with packaging materials to assure proper dosage delivery. Compounds of the invention that have poor skin permeability may require one or more permeation enhancers, whereas compounds rapidly absorbed through the skin may require formulation with absorption-retarding agents or barriers.

The term “pharmaceutically acceptable topical carrier” refers to a carrier medium, suitable for topical application, that provides appropriate delivery of an effective amount of a compound of the invention, such as an inactive liquid or cream vehicle capable of suspending or dissolving the compound. The skilled person will appreciate that this term encompasses carrier materials approved for use in topical cosmetics as well.

The terms “permeation enhancer” relates to an increase in the permeability of the skin, nail, hair, claw or hoof to the compound of the invention, so as to increase the rate and extent of permeation of the compound. The enhanced permeation can be observed, for example, by measuring the rate of diffusion of the drug through animal or human skin, nail, hair, claw or hoof using a diffusion cell apparatus. A diffusion cell is described by Merritt et al. Diffusion Apparatus for Skin Penetration, J of Controlled Release, 1 (1984) pp. 161-162.

The ointments, pastes, creams, lotions, gels, suppositories, powders, solutions, sprays, drops, inhalants and patches for topical administration may contain, in addition to a compound of the invention, one or more pharmaceutically acceptable excipients, such animal or vegetable fats, oils, waxes, paraffins, starch, tragacanth, cellulose derivatives, polyethylene glycols, silicones, bentonites, silicic acid, talc, zinc oxide, preservatives, antioxidants, fragrances, emulsifiers, dyes, inert fillers, anti-irritants, tackifiers, fragrances, opacifiers, antioxidants, gelling agents, stabilizers, surfactants, emollients, coloring agents, preservatives, buffering agents, permeation enhancers. Such excipients should not interfere with the effectiveness of the biological activity of the active agent and not be deleterious to the epithelial cells or their function.

Transdermal administration may be achieved by means of a transdermal patch. The transdermal patch may be of the ‘reservoir and porous membrane’ type or employ a ‘matrix system’.

The solubility of compounds of compounds of the invention used in the preparation of pharmaceutical compositions may be increased by the use of appropriate formulation techniques, such as the incorporation of solubility-enhancing agents.

Pharmaceutical compositions may be formulated to be immediate and/or modified release. Conveniently compounds of the invention are formulated for immediate release

Modified release formulations include delayed-, sustained-, pulsed-, controlled-, targeted- and programmed-release. Thus compounds of the invention may be formulated as a solid, semi-solid, or thixotropic liquid for administration as an implanted depot providing modified release of the active compound. Examples of such formulations include poly(dl-lactic-coglycolic)acid (PGLA) microspheres.

The compounds of the invention may be combined with soluble macromolecular entities, such as cyclodextrin and suitable derivatives thereof, or polyethylene glycol-containing polymers, in order to improve their solubility, dissolution rate, taste-masking, bioavailability and/or stability for use in any of the aforementioned modes of administration.

For administration to human patients, the total daily dose of the compounds of the invention is typically in the range 1 mg to 10 g, such as 60 mg to 6 g, for example 100 mg to 1.5 g, depending on the mode of administration and efficacy. For example, administration may require a total daily dose of from 200 mg to 1 g, such as from 250 mg to 750 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e. inhibition of ITK. More particularly, the compounds of the invention are of use in the treatment of disorders for which an ITK inhibitor is indicated.

Preferably the animal is a mammal, more preferably a human.

Preferably the compound of the invention also inhibits TRKA.

In a further aspect of the invention there is provided a compound of the invention for use as a medicament.

In a further aspect of the invention there is provided a compound of the invention for use in the treatment of a disorder for which an ITK inhibitor is indicated.

In a further aspect of the invention there is provided use of a compound of the invention for the preparation of a medicament for the treatment of a disorder for which an ITK inhibitor is indicated.

In a further aspect of the invention there is provided a method of treating a disorder in an animal (preferably a mammal, more preferably a human) for which an ITK inhibitor is indicated, comprising administering to said animal a therapeutically effective amount of a compound of the invention.

Disorders or conditions for which an ITK inhibitor is indicated include inflammatory, autoimmune, dermatologic, eye, respiratory, joint, cardiovascular and neuroinflammatory diseases. The skilled person will appreciate that a given disease, disorder or condition may fall into more than one of the above categories.

More particularly, disorders or conditions for which an ITK inhibitor is indicated include:
inflammatory disorders, such as allergic conjunctivitis, celiac diseases, proctitis, eosinophilic gastroenteritis, mastocytosis, inflammatory bowel disease (e.g. Crohn's disease, ulcerative colitis, microscopic colitis (such as collagenous colitis or lymphocytic colitis), diversion colitis, Behcet's disease, and indeterminate colitis), nephritis, retinitis, retinopathy, myositis, vasculitis, Sjogren's syndrome, Wegener's granulomatosis, arteritis, sclerosing cholangitis, and eosinophilic esophagitis;
autoimmune disorders, such as lupus nephritis, autoimmune hepatitis, myasthenia gravis, Guillain-Barre syndrome, and Graves' disease;
eye disorders or conditions, including autoimmune diseases of the eye, keratoconjunctivitis, vernal conjunctivitis, non-infectious uveitis (e.g. uveitis associated with Behcet's disease and lens-induced uveitis), keratitis (e.g. herpetic keratitis and conical keratitis), corneal epithelial dystrophy, keratoleukoma, ocular premphigus, Mooren's ulcer, scleritis, retinitis, retinopathy, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization;
dermatological conditions, such as eczema (e.g. chronic and dyshidrotic eczema), chronic itch, dermatitis (e.g. atopic, irritant contact, allergic contact, occupational, perioral, stasis, nummular, seborrheic, xerotic, eyelid, diaper, and hand dermatitis), vitiligo, alopecia areata, pruritis (e.g. chronic idiopathic pruritus), psoriasis (e.g. plaque, guttate, inverse, pustular, nail, flexural palmoplantar, facial or erythrodermic psoriasis), scleroderma, pemphigus, dermatomyositis, neurodermatitis, skin flushing, urticaria, cutaneous lupus erythematosus (e.g. acute cutaneous lupus (acute skin lupus), subacute cutaneous lupus (subacute lupus), and chronic cutaneous lupus (discoid lupus)), keloid, sunburn, hypertrophic scar, idiopathic thrombocytopenic thrombotic purpura (also known as immune thrombocytopenia purpura (ITP)), ichthyosis (e.g. ichthyosis vulgaris), epidermal hyperplasia, acne, lichen planus, lichen sclerosis, rosacea, epidermolysis bullosa, intertrigo, keratosis pilaris, urticaria (e.g. chronic spontaneous urticaria, chronic idiopathic urticaria, chronic physical urticaria), molluscum contagiosum, Netherton syndrome, Vogt-Koyanagi-Harada syndrome, Sweet's syndrome, pityriasis alba, vulvovaginitis, Sutton's nevus/nevi, post inflammatory hypopigmentation, senile leukoderma, chemical/drug-induced leukoderma, palmoplantar pustulosis, pemphigoid, and hidradenitis suppurativa;
respiratory conditions, such as rhinitis (e.g. allergic and perennial rhinitis), rhinorrhoea, nasal congestion, nasal inflammation, asthma (e.g. chronic asthma, inveterate asthma, late asthma, bronchial asthma, allergic asthma, intrinsic asthma, extrinsic asthma, and dust asthma), chronic obstructive pulmonary disease (COPD), chronic and acute bronchoconstriction, chronic bronchitis, emphysema, chronic eosinophilic pneumonia, acute lung injury (ACI), adult respiratory distress syndrome (ARDS), pulmonary vascular disease (PVD), pulmonary arterial hypertension (PAH), bronchiectasis, sinusitis, pulmonary sarcoidosis, and silicosis;
joint disorders, such as arthritis (e.g. osteoarthritis, as well as psoriatic, rheumatoid, juvenile, and gouty arthritis), spondyloarthropathy (e.g. reactive arthritis (also known as Reiter's Syndrome) and axial spondyloarthritis (including ankylosing spondylitis)), cartilage inflammation, bone degradation, and Still's disease;
cardiovascular and metabolic disorders, such as diabetes (type 1 and type 2), diabetic neuropathy, cachexia, and Celiac Sprue; and
neuroinflammatory disorders, such as lupus (e.g. CNS, systemic and discoid lupus), diabetic neuropathy, and multiple sclerosis.

Allergic contact dermatitis (ACD) is a contact dermatitis characterised by an allergic response to contact with a substance. An example of ACD is urushiol-induced contact dermatitis (also called toxicodendron dermatitis or rhus dermatitis), which is caused by the oil urushiol found in various plants, including poison ivy, poison oak, poison sumac and the Chinese lacquer tree. Other allergens that can induce ACD include chromium, gold and nickel.

Irritant contact dermatitis (ICD) is a form of contact dermatitis that can be divided into forms caused by chemical irritants and those caused by physical irritants. Common chemical irritants include acids, alkalis, latex, oils, perfumes and preservatives in cosmetics, solvents, and surfactants.

Occupational dermatitis is an ACD or ICD arising from exposure to an allergen or irritant in a work environment.

Additionally, an ITK inhibitor may be of use in treating certain viral and bacterial infections, transplant rejection, septic shock, acute or chronic graft-versus-host disease, polymyalgia rheumatica, sarcoidosis, Addison's disease and Raynaud's syndrome.

In one embodiment the disorder or condition for which an ITK inhibitor is indicated is a dermatological condition. In another embodiment the dermatological condition for which an ITK inhibitor is indicated is dermatitis. In another embodiment the dermatitis for which an ITK inhibitor is indicated is atopic dermatitis.

A compound of the invention may usefully be combined with one or more other pharmacologically active compounds. Such combinations offer the possibility of significant advantages, including patient compliance, ease of dosing and synergistic activity.

In a further aspect of the invention there is provided a compound of the invention in combination with another pharmacologically active compound, or with two or more other pharmacologically active compounds.

In such combinations the compound of the invention and other pharmacologically active compound(s) may be administered simultaneously, such as in a single dosage form (e.g. a composition for topical administration, such as a cream or an ointment), sequentially or separately.

The one or more additional therapeutic agents may be selected from any of the agents or types of agent that follow:
an agent for treating autoimmune and/or inflammatory disorders, such as, sulfasalazine, mesalazine, azathioprine, an antibody (e.g. infliximab, adalimumab, belimumab, tanezumab, ranibizumab, bevacizumab, mepolizumab certolizumab, natalizumab, and vedolizumab), 6-mercaptopurine, hydroxychloroquine, mofetil, sodium mycophenolate, leflunomide, rituxan, solumedrol, depomedrol, a non-steroidal anti-inflammatory drug (NSAID) (e.g. aspirin, ibuprofen, celecoxib, valdecoxib, WBI-1001 and MRX-6), and a corticosteroid (e.g. betamethasone, dexamethasone, and prednisone);
an agent for treating dermatological conditions, such as an immunosuppressant (e.g. cyclosporin, tacrolimus, and pimecrolimus), an antibody (e.g. infliximab, adalimumab dupilumab, omalizumab, and efalizumab), a TNF inhibitor (e.g. etanercept), a PDE4 inhibitor (e.g. crisaborole), and a topical corticosteroid (e.g. fluocinonide, mapracorat, hydrocortisone, desonide, alclometasone, triamcinolone, and desoximetasone);
an agent for treating respiratory conditions, such as oxymetazoline, rifampin, an anti-histamine (e.g. fexofenadine, loratidine, desloratidine, levocetirizine, methapyrilene, cetirizine), a leukotriene receptor antagonist (e.g. montelukast and zafirlukast), a 5-lipoxygenase activating protein (FLAP) antagonist, a muscarinic receptor antagonist (e.g. tiotropium and ipratropium), sodium cromoglycate, sodium nedocromil, a corticosteroid (e.g. budesonide, fluticasone, mometasone, dexamethasone, prednisolone, ciclesonide, and beclomethasone), a beta-2 agonist (e.g. salmeterol, albuterol, salbutamol, fenoterol, and formoterol), and an antibody (e.g. omalizumab);
an agent for treating joint disorders, such as methotrexate, azathioprine, and an NSAID (e.g. aspirin, ibuprofen, celecoxib, valdecoxib, WBI-1001 and MRX-6);
an agent for treating cardiovascular and metabolic disorders, such as ursodeoxycholic acid, chloroquine, quinacrine, methylnorephrine, phenylephrine, methoxamine, oxymetazoline, theophylline, a PDE5 inhibitor (e.g. sildenafil, vardenafil, and tadalafil), a PDE4 inhibitor (e.g. crisaborole, ibudilast, cilomilast, roflumilast, and ampremilast), and a kinin B₁ or B₂ receptor antagonist; and
an agent for treating neuroinflammatory disorder treatments, such as cyclophosphamide.

The one or more additional therapeutic agents may also be selected from any of the agents that follow:
a JAK inhibitor, such as abrocitinib, baricitinib, brepocitinib cerdulatinib, decernotinib, delgocitinib, fedratinib, filgotinib, gandotinib, ilginatinib, itacitinib, lestaurtinib, momelotinib, oclacitinib pacritinib, peficitinib, ritlecitinib, ruxolitinib, tofacitinib, upadacitinib, ATI-502, BMS-986165, JTE052, PF-06826647, SNA-152, and SHR-0302;
an aryl hydrocarbon receptor agonist such as, tapinarof;
an IRAK4 inhibitor such as PF-06650833;
a vitamin D analog, such as calcipotriene;
a retinoic acid derivative such as, alitretinoin;
a liver X receptor (LXR) selective agonist, such as VTP-38543;
an H4 receptor antagonist, such as, ZPL-389;
an NKI receptor antagonist, such as, aprepitant and tradipitant;
a CRTH2 receptor antagonist, such as, fevipiprant and OC-459;
a chymase inhibitor, such as SUN 13834;
a GATA-3 inhibitor, such as SB-011 and GR-MD-02;
an ROR inverse agonist, such as VTP-43742, ARN6039, TAK-828 and JTE-451;
an immunomodulator, such as PF-06763809; and
an inhibitor of SYK and BTK, including but not limited to, R-348, fostamatinib, mastinib, mivavotinib, sperbrutinib, fenebrutinib, cerdulatinib, ibrutinib, entospletinib and tirabrutinib.

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms (e.g. topical, oral, parenteral, etc.), for administering the separate compositions at different dosage intervals, or for titrating the separate compositions against one another.

To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which an ITK inhibitor is indicated.

It is to be appreciated that all references herein to treatment include curative, palliative and prophylactic treatment.

In the non-limiting Examples and Preparations set out below that illustrate the invention, and in the aforementioned Schemes, the following the abbreviations, definitions and analytical procedures may be referred to:
AcOH is acetic acid;
Ac₂O is acetic anhydride;
APC is allophycocyanin;
aq. is aqueous;
atm is atmosphere;
ATP is adenosine 5′-triphosphate disodium salt trihydrate;
BINAP is (2,2′-bis(diphenylphosphino)-1,1′-binaphthyl);
Boc is tert-butoxycarbonyl;
BOC₂O is BOC anhydride, di-tert-butyl dicarbonate;
br is broad;
BTFFH is fluorobis(tetramethylene)formamidinium hexafluorophosphate;
BTK is Bruton's tyrosine kinase;
° C. is degrees celcius;
CBZ is carboxybenzyl (also known as benzyloxycarbonyl);
CD₃OD is deutero-methanol;
CDCl₃ is deutero-chloroform;
CMPI is 2-chloro-1-methylpyridinium iodide, Mukaiyama reagent;
conc. is concentrated;
CSA is camphor sulphonic acid;
δ is chemical shift;
d is doublet;
dd is doublet of doublets;
ddd is doublet of doublet of doublets;
ddq is doublet of doublet of quartets;
dt is doublet of triplets;
DAST is diethylaminosulfur trifluoride;
DCM is dichloromethane;
DCE is 1,2-dichloroethane;
Dess-Martin periodinane is 3-oxo-1,3-dihydro-1λ⁵,2-benziodoxole-1,1,1-triyl triacetate;
DHP is dihydropyran;
DMAP is 4-dimethylaminopyridine;
DMAC is dimethylacetamide;
DMF is N,N-dimethylformamide;
DMSO is dimethyl sulfoxide;
DPPA is diphenylphosphoryl azide;
EDCl is 1-ethyl-3-(3-dimethylaminopropyl)carbodiimide hydrochloride;
ee is enantiomeric excess;
eq is equivalent;
EDTA is ethylenediaminetetraacetic acid;
ESI-MS is electrospray ionization mass spectrometry;
EtOAc is ethyl acetate;
EtOH is ethanol;
EtONa is sodium ethoxide;
Et₃N is triethylamine;
Et₃SiH is triethylsilane;
g is gram;
h is hour(s);
HATU is 1-[bis(dimethylamino)methylene]-1H-1,2,3-triazolo[4,5-b]pyridinium 3-oxide, hexafluorophosphate;
HBTU is N,N,N′,N′-tetramethyl-O-(1H-benzotriazol-1-yl)uronium hexafluorophosphate;
HEPES is (4-(2-hydroxyethyl)-1-piperazineethanesulfonic acid);
HPLC is high pressure liquid chromatography;
iPr₂NEt is N,N-diisopropylethyl amine, also known as Hunig's base;
iPrOH is isopropanol, also known as 2-propanol;
Kg is kilogram;
KOAc is potassium acetate;
KOtBu is potassium tert-butoxide;
L is liter;
LCMS is liquid chromatography mass spectrometry;
LDA is lithium diisopropylamide;
LiHMDS is lithium hexamethyldisilazide, also known as lithium bis(trimethylsilyl)amide;
m is multiplet;
M is molar;
MeCN is acetonitrile;
MeNH₂ is methyl amine;
MeOH is methanol;
MHz is mega Hertz;
min is minutes;
mL is milliliter;
mm is millimeter;
mmol is millimole;
mol is mole;
MS m/z is mass spectrum peak;
MTBE is methyl tert-butyl ether;
n-BuLi is n-butyl lithium;
N, in the context of concentration, is normal
NaHMDS is sodium bis(trimethylsilyl) amide;
NaOtBu is sodium tert-butoxide;
NBS is N-bromosuccinimide;
NH₄OH is 33 M aq. ammonia;
NMP is N-Methyl-2-pyrrolidone;
NMR is nuclear magnetic resonance;
PCC is pyridinium chlorochromate;
PDC is pyridinium dichromate;
Pd₂(dba)₃ is tris(dibenzylideneacetone)dipalladium(0);
Pd/C is palladium on carbon;
Pd(dppf)Cl₂ is 1,1′-bis(diphenylphosphino)ferrocene]dichloropalladium(II);
Pd(OAc)₂ is palladium(II)acetate;
Pd(OH)₂/C is palladium(II)hydroxide on carbon;
Pd(Ph₃P)₄ is tetrakis(triphenylphosphine)palladium(0);
PE is petroleum ether;
PhCH₃ is toluene;
PMB is para-methoxybenzyl;
PSI is pounds per square inch;
pTSA is p-toluenesulfonic acid monohydrate;
q is quartet;
Qphos is 1,2,3,4,5-pentaphenyl-1′-(di-tert-butylphosphino)ferrocene;
quint is quintet;
RT is room temperature;
s is singlet;
sat. is saturated;
SEM-Cl is 2-(trimethylsilyl)ethoxymethyl chloride;
SFC is supercritical fluid chromatography;
SPhos is 2-Dicyclohexylphosphino-2′,6′-dimethoxybiphenyl;
t is triplet;
tt is triplet of triplets;
t-BuDavePhos is 2-di-tert-butylphosphino-2′-(N,N-dimethylamino)biphenyl;
t-BuOH is tert-butanol;
TCEP is tris(2-carboxyethyl)phosphine;
TCFH is N,N,N′,N′-tetramethylchloroformamidinium hexafluorophosphate;
TEMPO is tetramethylpiperidine N-oxyl;
TFA is trifluoroacetic acid;
TFAA is trifluoroacetic anhydride;
TGA is thermogravimetric analysis;
THF is tetrahydrofuran;
TMSCF₃ is trifluoromethyltrimethylsilane;
TMSOTf is trimethylsilyl trifluoromethanesulfonate;
T₃P is propylphosphonic anhydride;
Tris is tris(hydroxymethyl)aminomethane;
μL is microliter;
μm is micrometer;
μmol is micromole;
v/v is volume by volume;
w/v is volume by volume;
XantPhos is 4,5-Bis(diphenylphosphino)-9,9-dimethylxanthene; and
ZnEt₂ is diethylzinc.

Unless otherwise stated all reactions are run under a nitrogen atmosphere. RT (room temperature) is generally taken to mean approximately 22° C. (±5° C.). Unless otherwise stated, the term “concentrated” refers to the process of removal of volatile compounds such as solvents by use of a rotary evaporator under reduced pressure.

¹H NMR spectra were in all cases consistent with the proposed structures. Characteristic δ for ¹H-NMR are reported relative to residual solvent signals (for CDCl₃, δH=7.27 ppm; for DMSO-d₆, δH=2.50 ppm, for CD₃OD, δH=3.30 ppm) using conventional abbreviations for designation of major peaks. The skilled person will appreciate that tautomers may be recorded within the NMR data and some exchangeable protons may not be visible. Likewise, the skilled person will appreciate that a mixture of rotamers may be recorded within the NMR data.

Mass spectra were recorded using either ESI-MS. Where relevant and unless otherwise stated the m/z data provided are for isotopes ¹⁹F, ³⁵Cl, ⁷⁹Br and/or ⁸¹Br.

The term “chromatography” refers to silica gel chromatography with mobile phase consisting of mixtures or gradients of either EtOAc/heptane or methanol/DCM or some combination thereof.

Where silica gel chromatography, preparative HPLC or SFC chromatography have been used, the skilled person will appreciate that any suitable solvent or solvent combination may be employed to purify the desired compound.

Nomenclature for the compounds of the Preparations and Examples that follow was generated using ChemDraw Professional 19.0, Perkin Elmer, in accordance with the IUPAC (International Union of Pure and Applied Chemistry).

### Preparations

### Preparation 1

### 1-Methoxy-5-methylcvclohexa-1,4-diene (Compound 2)

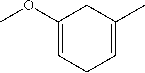

Anhydrous ammonia (1.40 kg, 82.2 mol) was bubbled into a solution of 1-methoxy-3-methylbenzene (Compound 1, 500 g, 4.09 mol) in t-BuOH (1.50 L) and THF (1.0 L) at about −55° C. Lithium sand (62.5 g, 9.0 mol) was added to the mixture while maintaining the temperature between about −50° C. and −60° C. After the addition, the reaction mixture was stirred between about −50° C. and −60° C. for about 2 h and then gradually warmed to about 15° C. The ammonia was allowed to evaporate and mixture was treated with NH₄Cl (500 g, 9.35 mol) followed by water (500 mL). The organic layer was separated and the aqueous layer was extracted with EtOAc (2×500 mL). The combined EtOAc layers were washed with brine, dried (Na₂SO₄), filtered and concentrated to provide the title compound. Yield: 404 g (80%). ¹H NMR (400 MHz, CDCl₃) δ=5.41 (tt, 1H), 4.67-4.60 (m, 1H), 3.56 (s, 3H), 2.77 (ddq, 2H), 2.65-2.56 (m, 2H), 1.70 (dq, 3H).

### Preparation 2

### 7-Methyl-1,4-dioxaspiro[4.5]dec-7-ene

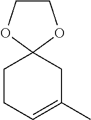

A solution of Compound 2 (500 g, 4.03 mol) in DCM (4.0 L) was treated with (±)-10-camphorsulfonic acid (46.8 g, 201 mmol) and ethylene glycol (399 g, 6.04 mol) between about −10° C. and 0° C. The mixture was stirred at about 0° C. for about 30 min. The reaction mixture was washed sequentially with sat. aq. NaHCO₃ (2 L) and then water (2×2 L), dried (Na₂SO₄), filtered and concentrated to provide the title compound. Yield: 621 g (100%). ¹H NMR (400 MHz, CDCl₃) δ=5.38 (td, 1H), 3.95 (t, 4H), 2.21-2.12 (m, 6H), 1.67 (d, 3H).

### Preparation 3

### 1-Methylspiro[bicyclo[4.1.0]heptane-3,2′-[1,3]dioxolane]

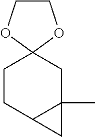

A solution of ZnEt₂ (1.00 M, 3.89 L) in DCM (2.0 L) was cooled to about 0° C. and treated with TFA (444 g, 3.89 mol, 288 mL) dropwise at about 0° C. The mixture was stirred at about 0° C. for about 30 min after which CH₂I₂ (314 mL, 3.89 mol) was added dropwise at about 0° C. The mixture was stirred at about 0° C. for about 30 min. Preparation 2 (300 g, 1.95 mol) was added dropwise, maintaining the temperature at about 0° C. and the resulting mixture was stirred at about 0° C. for about 30 min. The reaction mixture was poured into water (1 L) and extracted with DCM (3×1.75 L). The combined organic phases were dried (Na₂SO₄), filtered and concentrated to provide the title compound. Yield: 300 g (92%). ¹H NMR (400 MHz, CDCl₃) δ=3.78-3.93 (m, 4H), 2.01-2.13 (m, 1H), 1.79-1.84 (m, 1H), 1.67-1.75 (m, 2H), 1.36-1.47 (m, 1H), 1.27 (m, 1H), 1.03 (s, 3H), 0.61-0.73 (m, 1H), 0.25-0.35 (m, 2H).

### Preparation 4

### 1-Methylbicyclo[4.1.0]heptan-3-one

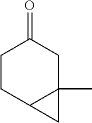

A solution of Preparation 3 (300 g, 1.78 mol) in THF (1.5 L) and water (300 mL) was treated with pTSA·H₂O (34.0 g, 178 mmol). The mixture was stirred at about 60° C. for about 3 h, cooled to RT and treated with sat. aq. NaHCO₃ solution until the pH was between 6-7. The mixture was extracted with MTBE (3×500 mL), dried (Na₂SO₄), filtered and concentrated. The crude product was purified by chromatography to provide the title compound. Yield: 85.7 g (39%). ¹H NMR (400 MHz, CDCl₃) δ=2.60-2.40 (m, 2H), 2.32-2.18 (m, 2H), 2.11-1.93 (m, 2H), 1.13 (s, 3H), 1.03-0.94 (m, 1H), 0.48-0.39 (m, 2H).

### Preparation 5

### Ethyl 2-(6-methyl-4-oxobicyclo[4.1.0]heptan-3-yl)-2-oxoacetate (5)

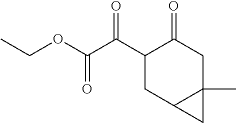 [SMILES: CCOC(=O)C(=O)C1CC2CC2(C)CC1=O]

A solution of Preparation 4 (200 g, 1.61 mol) in ethanol (1.0 L) was treated with sodium ethoxide (126 g, 1.77 mol) at about 0° C. Diethyl oxalate (259 g, 1.77 mol, 242 mL) was added at about 0° C. and the mixture was gradually warmed to RT and stirred for about 1 h. The mixture was poured into 1 N HCl (1.25 L) and extracted with DCM (3×1 L). The combined organic extracts were dried (Na₂SO₄), filtered and concentrated. The crude product was purified by chromatography to provide the title compound. Yield: 350 g (97%). LC/MS m/z (M+H)⁺=225.1

### Preparation 6

### Ethyl 5a-methyl-1,4,4a,5,5a,6-hexahydrocyclopropa[f]indazole-3-carboxylate

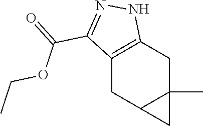 [SMILES: CCOC(=O)c1n[nH]c2c1CC1CC1(C)C2]

To a solution of Preparation 5 (350 g, 1.56 mol) in ethanol (1.5 L), was added hydrazine hydrate (79.7 g, 1.56 mol) at about 0° C. The resulting mixture was stirred at RT for about 2 h. The mixture was treated with H₂O (2.5 L) and extracted with DCM (3×2 L). The combined organic layers were dried (Na₂SO₄), filtered and concentrated. The crude product was purified by chromatography to provide the title compound. Yield: 200 g (58%). ¹H NMR (400 MHz, CDCl₃) δ=8.95 (s, 1H), 4.36 (q, 2H), 3.30-3.21 (m, 1H), 3.06 (d, 1H), 2.94 (dd, 1H), 2.71 (dd, 1H), 1.38 (t, 3H), 1.24 (s, 3H), 1.09-0.99 (m, 1H), 0.37-0.29 (m, 1H), 0.18 (t, 1H); LC/MS m/z (M+H)⁺=221.1.

### Preparations 6a and 6b

### Ethyl (4aR,5aS)-5a-methyl-1,4,4a,5,5a,6-hexahydrocyclopropa[f]indazole-3-carboxylate (6a) and ethyl (4aS,5aR)-5a-methyl-1,4,4a,5,5a,6-hexahydrocyclopropa[f]indazole-3-carboxylate (6b)

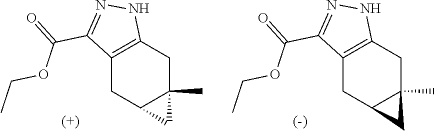

Preparation 6 was separated by chiral SFC (Chiral Tech OZ-H 250 mm×4.6 mm, 5 μm column with a mobile phase of 20% methanol (0.2% v/v 7 M NH₃/methanol) and 80% CO₂; flow rate 3.0 mL/min) to provide the title compounds.

Preparation 6a: retention time=3.89 min, 100% ee, [α]²⁰_{D}=+67.1 (c=4.2, methanol); LC/MS m/z (M+H)⁺=221.1.

Preparation 6b: retention time=4.76 min, 98.9% ee; [α]²⁰_{D}=−80.2 (c=4.7, methanol); ¹H NMR (400 MHz, CDCl₃) δ=4.33 (q, 2H), 3.46 (s, 2H), 3.27-3.18 (m, 1H), 3.04 (d, 1H), 2.91 (dd, 1H), 2.72-2.63 (m, 1H), 1.34 (t, 3H), 1.21 (s, 3H), 1.07-0.96 (m, 1H), 0.34-0.26 (m, 1H), 0.16 (t, 1H); LC/MS m/z (M+H)⁺=221.1.

### Preparations 7a and 7b

### Ethyl (4aS,5aR)-5a-methyl-2-((2-(trimethylsilyl)ethoxy)methyl)-2,4,4a,5,5a,6-hexahydrocyclopropa[f]indazole-3-carboxylate (7a) and ethyl (4aS,5aR)-5a-methyl-1-((2-(trimethylsilyl)ethoxy)methyl)-1,4,4a,5,5a,6-hexahydrocyclopropa[f]indazole-3-carboxylate (7b)

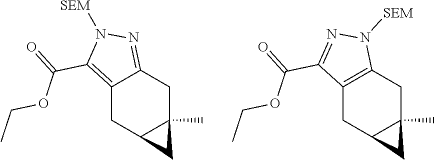

A suspension of sodium hydride (60% suspension, 19.3 g, 483 mmol) in THF (500 mL) was treated with a solution of Preparation 6b (103 g, 467.6 mmol) in THF (1.25 L) at about 0° C. After about 30 min, SEM-Cl (81.9 g, 491 mmol) was added at about 0° C. and the mixture was stirred at about 0° C. for about 3 h. The mixture was treated with sat. aq. NH₄Cl (500 mL) at about 0° C. The mixture was extracted with EtOAc (3×500 mL), washed with brine (500 mL), dried (MgSO₄), filtered and concentrated. The crude product was purified by chromatography to provide the title compounds.

Preparation 7a: Yield: 5.5 g (3.3%);¹H NMR (400 MHz, DMSO-d₆) δ=5.63 (s, 2H), 4.27 (qt, 2H), 3.53-3.41 (m, 2H), 3.18 (d, 1H), 2.97-2.84 (m, 2H), 2.64 (d, 1H), 1.30 (t, 3H), 1.21 (s, 3H), 1.05 (dt, 1H), 0.78-0.68 (m, 2H), 0.31 (dd, 1H), 0.02 (d, 1H), −0.11 (s, 9H); LC/MS m/z (M+H)⁺=351.3

Preparation 7b: Yield: 138 g (84%); ¹H NMR (400 MHz, CDCl₃) δ=5.48-5.30 (m, 2H), 4.37 (q, 2H), 3.59-3.41 (m, 2H), 3.23 (dd, 1H), 3.07 (d, 1H), 3.02-2.86 (m, 1H), 2.73-2.58 (m, 1H), 1.36 (t, 3H), 1.23 (s, 3H), 1.13-0.95 (m, 1H), 0.85 (ddt, 2H), 0.42-0.27 (m, 1H), 0.18 (t, 1H), −0.05 (s, 9H); LC/MS m/z (M+H)⁺=351.3; [α]²⁰_{D}=−30.9 (c=1, methanol)

### Preparation 8

### ((4aS,5aR)-5a-Methyl-1-((2-(trimethylsilyl)ethoxy)methyl)-1,4,4a,5,5a,6-hexahydrocyclopropa[f]indazol-3-yl)methanol

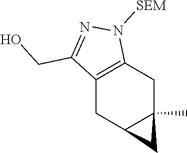 [SMILES: C[C@@]12Cc3c(c(CO)nn3COCC[Si](C)(C)C)C[C@@H]1C2]

A suspension of LiAlH₄ (14.94 g, 393.7 mmol) in THF (500 mL) at about 0° C. was treated dropwise with a solution of Preparation 7b (138 g, 393.7 mmol) in THF (1 L). The mixture was stirred at about 15° C. for about 2 h. The mixture was cooled to about 0° C. and treated sequentially by dropwise addition of H₂O (15 mL), 15% aq. NaOH (15 mL) and H₂O (30 mL), followed by MgSO₄. The resultant mixture was stirred for about 30 min, diluted with EtOAc (500 mL) and filtered. The filtrate was concentrated to provide the title compound. Yield: 110 g (91%). ¹H NMR (400 MHz, CDCl₃) δ=5.35-5.23 (m, 2H), 4.59 (dd, 2H), 3.51 (t, 2H), 3.03 (d, 1H), 2.86-2.81 (m, 2H), 2.64 (d, 1H), 1.90 (t, 1H), 1.24 (s, 3H), 1.04 (dq, 1H), 0.88 (td, 2H), 0.36 (dd, 1H), 0.23 (t, 1H), −0.03 (d, 9H); LC/MS m/z (M+H)+=309.3; [α]²⁰_{D}=−18.3 (c=1, methanol).

### Preparation 9

### (4aS,5aR)-5a-Methyl-1-((2-(trimethylsilyl)ethoxy)methyl)-1,4,4a,5,5a,6-hexahydrocyclopropa[f]indazole-3-carbaldehyde (9)

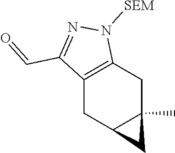 [SMILES: C[C@@]12Cc3c(c(C=O)nn3COCC[Si](C)(C)C)C[C@@H]1C2]

A solution of Preparation 8 (110.13 g, 0.36 mol) in DCM (1.5 L) was treated with activated MnO₂ (310 g, 3.57 mol) and the resulting mixture was stirred at RT for about 16 h. The mixture was filtered through a pad of Celite©. The filtrate was concentrated and the crude product was purified by chromatography to provide the title compound. Yield: 96 g (88%). ¹H NMR (400 MHz, DMSO-d₆) δ=9.86 (s, 1H), 5.52-5.38 (m, 2H), 3.50 (dd, 2H), 3.11 (dd, 2H), 2.93-2.82 (m, 1H), 2.67 (d, 1H), 1.22 (s, 3H), 1.08-1.02 (m, 1H), 0.81 (td, 2H), 0.39 (dd, 1H), 0.08 (t, 1H), −0.07 (s, 9H); LC/MS m/z (M+H)⁺=307.3; [α]²⁰_{D}=−38.9 (c=1, methanol).

### Preparation 10

### 7,7-Difluoro-1-methylspiro[bicyclo[4.1.0]heptane-3,2′-[1,3]dioxolane]

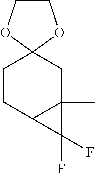

The following reaction was carried out in 26 batches in parallel. A solution of Compound 2 (150 g, 972 mmol) in THF (1.20 L) was treated with TMSCF₃ (276 g, 1.95 mol) and sodium iodide (75.8 g, 506 mmol). The mixture was stirred at about 70° C. for about 16 h. The 26 reaction mixtures were cooled to room temperature and combined. The mixture was diluted with water (10 L) and extracted with MTBE (4×3 L). The organic phase was washed with brine (8 L), dried (Na₂SO₄), filtered and concentrated to provide the title compound. Yield: 4.30 kg (83%).

### Preparation 11

### 7,7-Difluoro-1-methylbicyclo[4.1.0]heptan-3-one

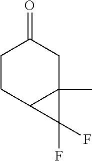

The following reaction was carried out in 5 batches in parallel. A mixture of Preparation (860 g, 4.21 mol) in THF (10 L) was treated with 3M HCl (2.6 L) at RT. The mixture was stirred at RT for about 16 hours. The 5 reaction mixtures were combined and extracted with MTBE (4×2.5 L), washed with sat. aq. NaHCO₃ (5 L) and brine (5 L). The organic phase was dried (Na₂SO₄), filtered and concentrated to provide the title compound. Yield: 3.50 kg. ¹H NMR (400 MHz, CDCl₃) δ=2.56 (br d, 1H), 2.38-2.13 (m, 4H), 2.01-1.89 (m, 1H), 1.49-1.38 (m, 1H), 1.27 (br s, 3H).

### Preparation 12

### Ethyl 2-(7,7-difluoro-6-methyl-4-oxobicyclo[4.1.0]heptan-3-yl)-2-oxoacetate

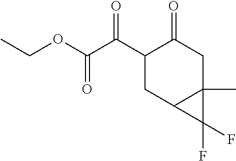 [SMILES: CCOC(=O)C(=O)C1CC2C(F)(F)C2(C)CC1=O]

The following reaction was carried out in 8 batches in parallel. A solution of Preparation 11 (250 g, 1.56 mol) in ethanol (1.25 L) was treated with sodium ethoxide (112 g, 1.65 mol) in portions at about 0° C. The resultant mixture was treated with diethyl oxalate (242 g, 1.65 mol) at about 0° C. The reaction mixture was stirred at RT for about 1 h. The 8 batches were combined. The mixture was poured into 3 M aq. HCl solution (8.00 L) and extracted with DCM (3×2 L). The organic extracts were washed with brine (5 L), dried (Na₂SO₄), filtered and concentrated to provide the title compound. Yield: 3.20 kg (98%).

### Preparation 13

### Ethyl 5,5-difluoro-5a-methyl-1,4,4a,5,5a,6-hexahydrocyclopropa[f]indazole-3-carboxylate

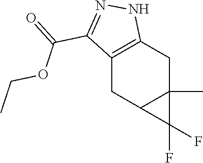 [SMILES: CCOC(=O)c1n[nH]c2c1CC1C(F)(F)C1(C)C2]

The following reaction was carried out 8 in batches in parallel. A suspension of Preparation 12 (400 g, 1.54 mol) in ethanol (2 L) was treated with hydrazine hydrate (76.9 g, 1.54 mol) at about 0° C. The reaction mixture was stirred at RT for about 16 h. The eight reaction mixtures were combined for workup. The reaction mixture was concentrated and the residue taken up in H₂O (5 L) and extracted with EtOAc (5×2 L). The combined organic extracts were dried (Na₂SO₄), filtered and concentrated. The crude product was triturated from 6:1 (v/v) EtOAc/ethanol (3 L) at RT to provide the title compound. Yield: 1.0 Kg. ¹H NMR (400 MHz, CDCl₃) δ=12.03-10.65 (br m, 1H), 4.38 (q, 2H), 3.30-3.04 (m, 3H), 2.79 (dd, 1H), 1.57 (br dd, 1H), 1.34-1.43 (m, 6H); LC/MS m/z (M+H)+=257.1.

### Preparations 14 and 15

### Ethyl (4aR,5aS)-5,5-difluoro-5a-methyl-1,4,4a,5,5a,6-hexahydrocyclopropa[f]indazole-3-carboxylate (14) and ethyl (4aS,5aR)-5,5-difluoro-5a-methyl-1,4,4a,5,5a,6-hexahydrocyclopropa[f]indazole-3-carboxylate (15)

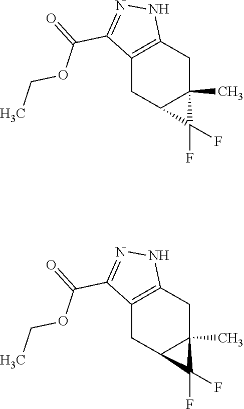

Preparation 13 was separated by chiral SFC using a Chiral Tech AD-H 250 mm×21.2 mm μm column with a mobile phase of CO_{2(g)}/methanol=80:20 with 0.2% 7N NH₃ in methanol and a flow rate of 200 mL/min.

SFC analytical method: Chiral Tech AD-H 250 mm×4.6 mm×5 μm A=CO₂; B=0.2% v/v NH₃ (as 7N NH₃ in methanol) in methanol; gradient=0-1 min 5% B, 1-9.5 min 5-60% B ramp; 9.5-10 min 60-5% B ramp.

Preparation 14: 100% ee by SFC analytical method, retention time=4.605 min

Preparation 15: 99.85% ee by SFC analytical method, retention time=5.565 min

### Preparation 16

### Ethyl (4aS,5aR)-5,5-difluoro-5a-methyl-1-((2-(trimethylsilyl)ethoxy)methyl)-1,4,4a,5,5a,6-hexahydrocyclopropa[f]indazole-3-carboxylate

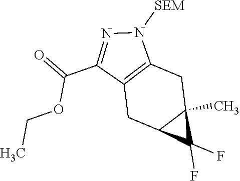 [SMILES: CCOC(=O)c1nn(COCC[Si](C)(C)C)c2c1C[C@@H]1C(F)(F)[C@]1(C)C2]

A mixture of sodium hydride (60% suspension, 4.25 g, 106 mmol) in THF (20 mL) cooled to about 0° C. was treated dropwise over about 45 min with a solution of Preparation 15 (18.16 g, 70.87 mmol) in THF (100 mL). The mixture was stirred at about 0° C. for about 1 h and then treated dropwise with SEM-Cl (17.7 g, 106 mmol) in THF (80 mL). The resultant mixture was stirred at RT for about 48 h. The reaction mixture was poured slowly over ice and extracted 3× with EtOAc. The organic extracts were combined, washed with brine, dried (MgSO₄), filtered and concentrated. The crude product was purified by chromatography (silica, EtOAc/heptane=0-20%) to provide 25.8 g (94%) of the title compound. ¹H NMR (400 MHz, CDCl₃) δ 5.50-5.40 (m, 2H), 4.40 (q, 2H), 3.55-3.47 (m, 2H), 3.26-3.05 (m, 3H), 2.76 (br dd, 1H), 1.62-1.54 (m, 1H), 1.44-1.35 (m, 6H), 0.94-0.81 (m, 2H), −0.03 (s, 9H); LC-MS m/z (M+H)⁺=387.4.

### Preparation 17

### ((4aS,5aR)-5,5-Difluoro-5a-methyl-1-((2-(trimethylsilyl)ethoxy)methyl)-1,4,4a,5,5a,6-hexahydrocyclopropa[f]indazol-3-yl)methanol

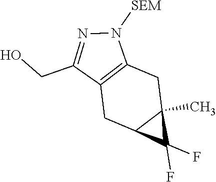 [SMILES: C[C@@]12Cc3c(c(CO)nn3COCC[Si](C)(C)C)C[C@@H]1C2(F)F]

A solution of Preparation 16 (25.84 g, 66.86 mmol) in THF (100 mL) at about −5° C. was treated with a solution of LiAlH₄ (100 mL, 1M in THF) dropwise at such a rate as to control the temperature between 0 and 10° C. The mixture was stirred at about 0° C. for about 1 h and gradually warmed to RT and stirred for about 4 h. The mixture was cooled to about −10° C. and treated dropwise with 6 N NaOH (45 mL) over about 30 min. EtOAc was added to aid stirring of the thick mixture and the slurry was warmed to RT. Anhydrous MgSO₄ was added and stirring continued for about 30 min. The mixture was filtered and the solids rinsed with EtOAc. The filtrate was concentrated to provide the title compound (21.89 g, 95%). ¹H NMR (400 MHz, CDCl₃) δ 5.38-5.21 (m, 2H), 4.60 (s, 2H), 3.55-3.41 (m, 2H), 3.08 (br d, 1H), 2.98-2.83 (m, 2H), 2.79-2.65 (m, 1H), 2.24 (br s, 1H), 1.60-1.49 (m, 1H), 1.40 (br s, 3H), 0.94-0.80 (m, 2H), −0.03 (s, 9H); LC-MS m/z (M+H)⁺=345.5.

### Preparation 18

### (4aS,5aR)-5,5-Difluoro-5a-methyl-1-((2-(trimethylsilyl)ethoxy)methyl)-1,4,4a,5,5a,6-hexahydrocyclopropa[f]indazole-3-carbaldehyde

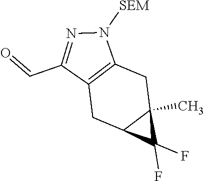 [SMILES: C[C@@]12Cc3c(c(C=O)nn3COCC[Si](C)(C)C)C[C@@H]1C2(F)F]

A solution of Preparation 17 (21.89 g, 63.55 mmol) in DCM (25 mL) was cooled to about 0° C. A solution of Dess-Martin periodinane (33.7 g, 79.4 mmol) in DCM (250 mL) was added dropwise at 0° C. over about 20 min. The mixture was treated with 2.2% water/DCM (50 mL) added dropwise over about 45 min at about 0° C. The mixture was warmed to RT and stirred for about 4 h. The mixture was treated with 1 N NaOH (380 mL) and stirred for about 30 min. The biphasic mixture was separated. The organic phase was washed with brine, dried (MgSO₄), filtered and concentrated. The crude product was purified by chromatography (silica, EtOAc/heptane=0-50%) to provide the title compound (17.8 g, 82%). ¹H NMR (400 MHz, CDCl₃) δ 9.98 (s, 1H), 5.52-5.35 (m, 2H), 3.61-3.43 (m, 2H), 3.27-3.05 (m, 4H), 2.82-2.66 (m, 1H), 1.42 (m., 3H), 0.98-0.80 (m, 2H), 0.08-−0.13 (m, 9H); LC-MS m/z (M+H)⁺=343.3.

### Preparation 19

### 6,6-Dimethyl-1-((2-(trimethylsilyl)ethoxy)methyl)-4,5,6,7-tetrahydro-1H-indazole-3-carbaldehyde and 6,6-dimethyl-2-((2-(trimethylsilyl)ethoxy)methyl)-4,5,6,7-tetrahydro-2H-indazole-3-carbaldehyde

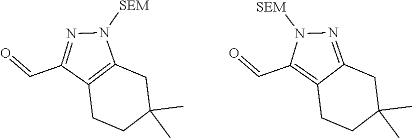 [SMILES: CC1(C)CCc2c(C=O)nn(COCC[Si](C)(C)C)c2C1 ; CC1(C)CCc2c(nn(COCC[Si](C)(C)C)c2C=O)C1]

### Step 1: 4,4-Dimethyl-2-oxocyclohexane-1-carbaldehyde (19a)

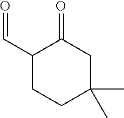

To a suspension of sodium hydride (60% suspension, 43.6 g, 1.09 mol) in THF (2.6 L) was added 3,3-dimethylcyclohexan-1-one (125 g, 991 mmol) at about 30° C. The reaction mixture was stirred for about 1 h. Ethyl formate (147 g, 1.98 mol) was added at about 35° C. and stirred for about 18 h at about 40° C. The reaction mixture was combined with an identical reaction using 125 g of 3,3-dimethylcyclohexan-1-one and similar proportions of other reagents. The mixture was acidified to about pH 3 with 1 M HCl. The mixture was extracted with EtOAc (2 L×2). The EtOAc extracts were combined and concentrated to provide the title compound (240 g, 79%). ¹H NMR (400 MHz, CDCl₃) δ=14.60-14.21 (m, 1H), 8.88-8.64 (m, 1H), 2.39 (t, 2H), 2.14 (s, 2H), 1.46 (t, 2H), 0.98 (s, 6H).

### Step 2: 6,6-Dimethyl-4,5,6,7-tetrahydro-1H-indazole (19b)

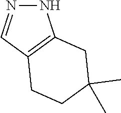

A solution of Preparation 19a (240 g, 1.27 mol) in methanol (2 L) was treated with hydrazine (76.0 g, 1.52 mol) at about 30° C. The reaction mixture was heated to about 70° C. and stirred for about 18 h. Water (100 mL) was added to the mixture. Most of the methanol was removed by rotary evaporation and the residue was extracted into EtOAc. The EtOAc extract was concentrated. The resulting residue was purified by chromatography to provide the title compound (150 g, 79%). ¹H NMR (400 MHz, CDCl₃) δ=7.33 (s, 1H), 2.56 (t, 2H), 2.43 (s, 2H), 1.53 (t, 2H), 1.02 (s, 6H); LC/MS m/z (M+H)⁺=150.8

### Step 3: 3-lodo-6,6-dimethyl-4,5,6,7-tetrahydro-1H-indazole (19c)

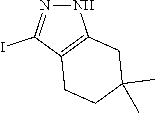

A solution of Preparation 19b (75.0 g, 0.5 mol) in DMF (350 mL) was treated with KOH (84 g, 1.5 mol) and iodine (253 g, 1 mol) at RT. The reaction mixture was stirred for about 18 h. A saturated solution of aqueous Na₂SO₃ (300 mL) was added to the reaction mixture. The mixture was combined with that of an identical reaction using 75 g of Preparation 19b and similar proportions of other reagents. The combined reaction mixtures were extracted into MTBE (2×1 L). The organic extracts were combined, dried (Na₂SO₄), filtered and concentrated. The resulting residue was purified by chromatography to provide the title compound (72 g, 26%). ¹H NMR (400 MHz, CDCl₃) δ=2.54-2.43 (m, 2H), 2.35 (t, 2H), 1.56 (t, 2H), 1.02 (s, 6H); LC/MS m/z (M+H)⁺=277.0.

### Step 4: 3-lodo-6,6-dimethyl-1-((2-(trimethylsilyl)ethoxy)methyl)-4,5,6,7-tetrahydro-1H-indazole and 3-iodo-6,6-dimethyl-2-((2-(trimethylsilyl)ethoxy)methyl)-4,5,6,7-tetrahydro-2H-indazole (19d)

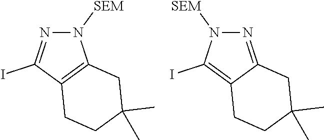

A solution of Preparation 19c (60 g, 217 mmol) in THF (600 mL) was treated with sodium hydride (60% suspension, 11.3 g, 282 mmol) at about 0° C. The suspension was stirred for about 30 min. The mixture was treated with SEM-Cl (42.5 mL, 239 mmol) at a temperature between 0-5° C. The reaction mixture was stirred at a temperature between 0-10° C. for about 2 h. A saturated aqueous solution of NH₄Cl (50 mL) was added at about 5° C. and the mixture was stirred for about 18 h. Water (100 mL) was added and the mixture was extracted with EtOAc (500 mL×2). The EtOAc extracts were combined and concentrated. The resulting residue was purified by chromatography to provide the title compounds (73 g, 83%) as a mixture of regioisomers. LC/MS m/z (M+H)⁺=407.1, RT=0.994 min.

### Step 5: 6,6-Dimethyl-1-((2-(trimethylsilyl)ethoxy)methyl)-4,5,6,7-tetrahydro-1H-indazole-3-carbaldehyde and 6,6-dimethyl-2-((2-(trimethylsilyl)ethoxy)methyl)-4,5,6,7-tetrahydro-2H-indazole-3-carbaldehyde

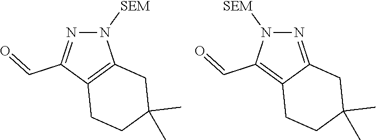 [SMILES: CC1(C)CCc2c(C=O)nn(COCC[Si](C)(C)C)c2C1 ; CC1(C)CCc2c(nn(COCC[Si](C)(C)C)c2C=O)C1]

A solution of Preparation 19d (73 g, 180 mmol) in DMF (900 mL) was treated with triethylsilane (41.8 g, 359 mmol), Et₃N (36.4 g, 359 mmol) and Pd(dppf)Cl₂ (13.1 g, 18.0 mmol). The reaction mixture was purged with CO_{(g)} and stirred at about 80° C. for about 16 h under a CO atmosphere (50 PSI). The reaction mixture was concentrated and the resulting residue was purified by column chromatography to provide the title compounds as a mixture (35 g, 63%). LC/MS m/z (M+H)⁺=309.1.

### Preparation 20

### (S)-2-Morpholinopropanoic acid

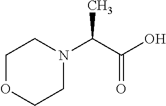

### Step 1: Benzyl (S)-2-morpholinopropanoate (20a)

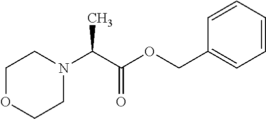 [SMILES: C[C@@H](C(=O)OCc1ccccc1)N1CCOCC1]

A solution of benzyl L-alaninate 4-methylbenzenesulfonate (500 g, 1.42 mol) in DMSO (3 L) was treated with Et₃N (624 g, 6.17 mol) and the mixture was cooled to about 0° C. A solution of 1-bromo-2-(2-bromoethoxy)ethane (429 g, 1.85 mol) in DMSO (1 L) was slowly added to the reaction. The resulting mixture was stirred at RT for about 36 h. Water (3 L) and EtOAc (2 L) were added to the mixture. The aqueous phase was extracted with EtOAc (2×1 L). The combined EtOAc extracts were dried (Na₂SO₄), filtered and concentrated. The crude product was purified by chromatography (silica, EtOAc/PE=11%) to provide the title compound (290 g, 82%). H NMR (400 MHz, DMSO-d₆) δ 7.42-7.31 (m, 5H), 5.19 (s, 2H), 3.79-3.66 (m, 4H), 3.35-3.30 (m, 1H), 2.69-2.56 (m, 4H), 1.32-1.29 (m, 3H).

### Step 2: (S)-2-Morpholinopropanoic acid

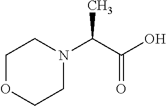

A solution of Preparation 20a (290 g, 1.56 mol) in methanol (2.9 L) was treated with 10% Pd(OH)₂/C (29 g) at RT. The mixture stirred under an atmosphere of H₂ for about 36 h. The solid was removed by filtration and the filtrate concentrated. The resulting residue was triturated with MTBE (200 mL×2) to provide the title compound (146 g, 79%). ¹H NMR (400 MHz, DMSO-d₆) δ 3.56 (s, 4H), 3.18-3.16 (m, 1H), 2.54-2.53 (m, 4H), 1.17-1.15 (m, 3H); LC/MS m/z (M+H)⁺=160.1; SFC analytical method: Chiral Tech IC 250 mm×4.6 mm, 5 μm, Gradient: 5 to 60% of 0.2% v/v (7 N NH₃ in methanol) in methanol/CO₂(g), 3.0 mL/min, retention time=5.88 min, 100% ee.

### Preparation 21

### (R)-2-(Tetrahydro-2H-pyran-4-yl)ProPanoic acid

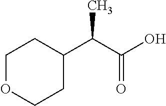

### Step 1: 2-(Tetrahydro-2H-pyran-4-yl)acetyl chloride (21a)

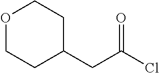

A solution of 2-(tetrahydro-2H-pyran-4-yl)acetic acid (17.3 g, 120 mmol) in DCM (400 mL) and DMF (1 mL) at about 0° C. was treated with oxalyl chloride (30.5 g, 240 mmol). The mixture was then warmed to RT and stirred for about 16 h. The mixture was concentrated to provide the title compound (19.5 g).

### Step 2: (R)-4-Benzyl-3-(2-(tetrahydro-2H-pyran-4-yl)acetyl)oxazolidin-2-one (21b)

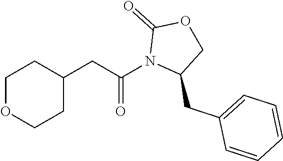 [SMILES: O=C(CC1CCOCC1)N1C(=O)OC[C@H]1Cc1ccccc1]

A solution of (R)-4-benzyloxazolidin-2-one (14.2 g, 79.9 mmol) in THF (500 mL) at about −78° C. was treated with 2.5 M n-BuLi in hexanes (48 mL). The mixture was then stirred at about −78° C. for about 2 h. A solution of Preparation 21a (19.5 g, 120 mmol) in THF (100 mL) was added at about −78° C. and the reaction mixture stirred for about 2 h. The mixture was then warmed to RT and stirred for about 16 h. The reaction mixture was treated with sat. aq. NH₄Cl (400 mL) and the resulting biphasic mixture was separated. The organic phase was dried (Na₂SO₄) and concentrated. The residue was suspended in DCM/PE (50 mL/300 mL) and stirred at −50° C. for 30 min. The solids were collected and dried to provide the title compound (21.5 g, 89%). LC/MS m/z (M+H)=303.8.

### Step 3: (R)-4-Benzyl-3-((R)-2-(tetrahydro-2H-pyran-4-yl)propanoyl)oxazolidin-2-one (21c)

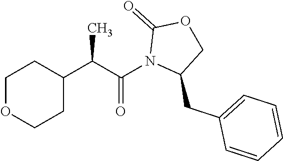 [SMILES: C[C@@H](C(=O)N1C(=O)OC[C@H]1Cc1ccccc1)C1CCOCC1]

A solution of Preparation 21b (21.5 g, 70.8 mmol) in THF (200 mL) at about −78° C. was treated with NaHMDS (1M in THF, 106 mL). The mixture was stirred for about 1 h and treated with methyl iodide (50.3 g, 354 mmol) at about −78° C. The reaction mixture was stirred at about −78° C. for about 2 h and then gradually warmed to RT over about 16 h. The reaction mixture was treated with sat. aq. NH₄Cl (300 mL) and the biphasic mixture was separated. The organic layer was washed with brine, dried (MgSO₄), filtered and concentrated. The crude product was purified by chromatography (silica, EtOAc/PE: 0-100%) to provide the title compound (15.5 g, 69%). ¹H NMR (400 MHz, CDCl₃) δ 7.32-7.11 (m, 5H), 4.62 (ddt, 1H), 4.20-4.07 (m, 2H), 4.02 (dd, 1H), 3.94 (dd, 1H), 3.65-3.54 (m, 1H), 3.33 (tt, 2H), 3.22 (dd, 1H), 2.72 (dd, 1H), 1.97-1.87 (m, 1H), 1.64-1.56 (m, 1H), 1.58-1.51 (m, 1H), 1.41-1.26 (m, 2H), 1.15 (d, 3H); LC/MS m/z (M+H)⁺=318.3.

### Step 4: (R)-2-(Tetrahydro-2H-pyran-4-yl)ProPanoic acid

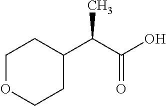

A solution of Preparation 21c (15.5 g, 48.8 mmol) in THF(410 mL) and H₂O (255 mL) at about 0° C. was treated with LiOH·H₂O (10.2 g, 244 mmol) and 30% aq. H₂O₂ (27.7 g, 244 mmol). The mixture was stirred at about 0° C. for about 1.5 h and then at RT for about 1.5 h. The mixture was treated with sat. aq. Na₂SO₃ (300 mL) and the organic solvent was removed under reduced pressure. The mixture was washed with DCM (2×200 mL) and these DCM washes were discarded. The aqueous phase was acidified to pH 1 with 12 N HCl. The mixture was extracted with DCM (3×200 mL), and these DCM extracts were combined, dried (Na₂SO₄), filtered and concentrated to provide the title compound (6.12 g, 79%). ¹H NMR (400 MHz, CDCl₃) δ 4.07-3.93 (m, 2H), 3.39 (dt, 2H), 2.30 (quint, 1H), 1.87-1.77 (m, 1H), 1.68-1.56 (m, 2H), 1.51-1.28 (m, 2H), 1.17 (d, 3H). [a]²⁰_{D}=−17.98 (c=0.3, ethanol); Chiral SFC (Chiral Tech IG 250×4.6 mm, 5 μm; 5-60% methanol/CO₂ gradient over 10 min): retention time −3.42 min, 97% ee.

### Preparation 22

### 2-Methyl-2-morpholinepropanoic acid

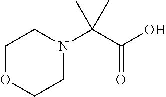

### Step 1: tert-Butyl 2-methyl-2-morpholinopropanoate (22a)

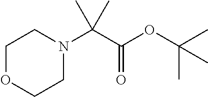 [SMILES: CC(C)(C)OC(=O)C(C)(C)N1CCOCC1]

A solution of tert-butyl 2-bromo-2-methylpropanoate (2.00 g, 8.97 mmol) in DMF (15 mL) was treated with K₂CO₃ (3.72 g, 26.9 mmol) and morpholine (0.94 g, 10.8 mmol) at about 10° C. The reaction mixture was stirred at about 60° C. for about 18 h. Water (20 mL) was added and the mixture was extracted into EtOAc (20 mL×3). The EtOAc extracts were combined, dried (Na₂SO₄), filtered, and concentrated. The resulting residue was purified by chromatography to provide the title compound (1.35 g, 66%). ¹H NMR (400 MHz, CDCl₃) δ=3.69 (t, 4H), 2.64 (t, 4H), 1.47 (s, 9H), 1.27 (s, 6H)

### Step 2: 2-Methyl-2-morpholinopropanoic acid

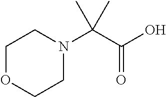

A solution of Preparation 22a (1.30 g, 5.67 mmol) in 1,4-dioxane (10 mL) was treated with 4 M HCl in 1,4-dioxane (2 mL). The reaction mixture was stirred at about 60° C. for about 15 h. The solvent was removed under reduced pressure to provide the title compound (790 mg, 81%). ¹H NMR (400 MHz, CD₃OD) δ=4.20-4.00 (m, 2H), 4.00-3.81 (m, 2H), 3.49-3.32 (m, 4H), 1.66 (s, 6H)

### Preparation 23

### (S)-3-Methyl-2-morpholinobutanoic acid

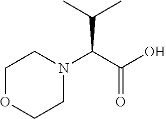

### Step 1: tert-Butyl (S)-3-methyl-2-morpholinobutanoate (23a)

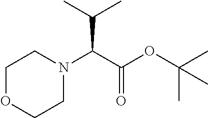 [SMILES: CC(C)[C@@H](C(=O)OC(C)(C)C)N1CCOCC1]

A solution of tert-butyl L-valinate (800 mg, 3.81 mmol) in DMSO (30 mL) was treated with Et₃N (2.7 mL, 19.1 mmol) at about 10° C. 1-Bromo-2-(2-bromoethoxy)ethane (0.97 g, 4.20 mmol) was added dropwise over a period of about 30 min. The reaction mixture was stirred for about 18 h. Water was added and the mixture was extracted into EtOAc (3×). The organic extracts were combined, dried (Na₂SO₄), filtered and concentrated. The crude product was purified by chromatography to provide the title compound (800 mg, 86%). ¹H NMR (400 MHz, CDCl₃) δ=3.71-3.60 (m, 4H), 2.69-2.50 (m, 5H), 2.07-1.92 (m, 1H), 1.49 (s, 9H), 0.97 (d, 3H), 0.89 (d, 3H)

### Step 2: (S)-3-Methyl-2-morpholinobutanoic acid

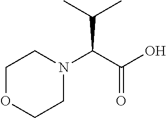

A solution of Preparation 23a (800 mg, 3.29 mmol) in 1,4-dioxane (5 mL) was treated with 4 M HCl in 1,4-dioxane (5 mL) at about 10° C. The reaction mixture was stirred at about 50° C. for about 18 h. The solvent was removed to provide the title compound (700 mg, 95%). ¹H NMR (400 MHz, CD₃OD) δ=4.05-3.88 (m, 5H), 3.56-3.34 (m, 4H), 2.63-2.49 (m, 1H), 1.19 (d, 3H), 1.05 (d, 3H)

### Preparation 24

### (S)-2-Morpholinobutanoic acid

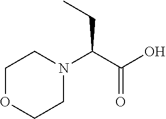

### Step 1: tert-Butyl (S)-2-morpholinobutanoate (24a)

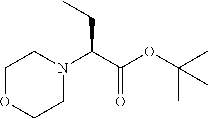 [SMILES: CC[C@@H](C(=O)OC(C)(C)C)N1CCOCC1]

A solution of tert-butyl (S)-2-aminobutanoate (1.5 g, 7.67 mmol) in DMSO (20 mL) was treated with Et₃N (5.3 mL, 38.3 mmol) and 1-bromo-2-(2-bromoethoxy)ethane (1.96 g, 8.43 mmol) at about 10° C. The reaction mixture was stirred for about 18 h. Water was added and the mixture was extracted into EtOAc (3×). The organic extracts were combined, dried over Na₂SO₄, filtered, and concentrated. The resulting residue was purified by chromatography to provide the title compound (1.20 g, 68%). ¹H NMR (400 MHz, CDCl₃) δ=3.77-3.61 (m, 4H), 2.94 (t, 1H), 2.72-2.53 (m, 4H), 1.81-1.68 (m, 1H), 1.68-1.59 (m, 1H), 1.49 (s, 9H), 0.93 (t, 3H).

### Step 2: (S)-2-Morpholinobutanoic acid

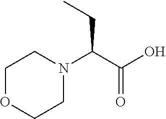

A solution of Preparation 24a (1.10 g, 4.80 mmol) in 1,4-dioxane (5 mL) was treated with 4 M HCl in 1,4-dioxane (5 mL) at about 10° C. The reaction mixture was stirred at about 50° C. for about 18 h. The solvent was removed under reduced pressure to provide the title compound (0.90 g, 90%). ¹H NMR (400 MHz, CD₃OD) δ=4.21-3.74 (m, 5H), 3.57-3.33 (m, 4H), 2.24-2.08 (m, 1H), 2.02 (d, 1H), 1.07 (t, 3H)

### Preparation 25

### tert-Butyl (5,6-diaminopyridin-3-yl)(methyl)carbamate

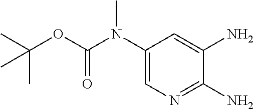 [SMILES: CN(C(=O)OC(C)(C)C)c1cnc(N)c(N)c1]

### Step 1: Methyl 6-(dibenzylamino)-5-nitronicotinate (25a)

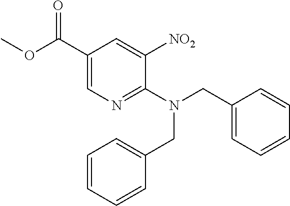 [SMILES: COC(=O)c1cnc(N(Cc2ccccc2)Cc2ccccc2)c([N+](=O)[O-])c1]

The following reaction was carried out in four parallel batches. A solution of methyl 6-chloro-5-nitronicotinate (375 g, 1.73 mol) in THF (3 L) was treated with Et₃N (482 mL, 3.46 mol) at RT. The resulting solution was cooled to about 5° C. Dibenzylamine (338 mL, 1.77 mol) was added dropwise at about 10-15° C. The reaction mixture was stirred at RT for about 16 h. The four reaction mixtures were concentrated individually and the residues were combined. The resulting material was diluted with EtOAc (8 L) and washed with sat. aq. NH₄Cl (3×1 L). The aqueous washes were combined and extracted with EtOAc (2×1 L). The combined EtOAc extracts were washed with brine (2 L), dried (Na₂SO₄), filtered, and concentrated to provide the title compound (2.80 Kg). ¹H NMR (400 MHz, CDCl₃) δ=8.93 (d, 1H), 8.64 (d, 1H), 7.37-7.21 (m, 5H), 7.12 (br dd, 5H), 4.68 (s, 4H), 3.91 (s, 3H).

### Step 2: 6-(Dibenzylamino)-5-nitronicotinic acid (25b)

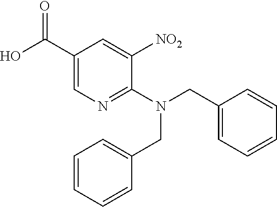 [SMILES: O=C(O)c1cnc(N(Cc2ccccc2)Cc2ccccc2)c([N+](=O)[O-])c1]

The following reaction was carried out in five parallel batches. A solution of Preparation 25a (656 g, 1.74 mol) in THF (1.3 L) and methanol (1.3 L) at RT was treated slowly with a solution of NaOH (146 g, 3.65 mol) in H₂O (650 mL). The resulting mixture was stirred at 50° C. for about 16 h and then cooled to a temperature between 10-15° C. The mixture was acidified to pH 3 with 2 N HCl. The five reaction mixtures were combined and the resulting suspension was filtered. The collected solids were dissolved in DCM (15 L), washed with brine (2 L), dried (Na₂SO₄), filtered, and concentrated to provide the title compound (3.1 kg). ¹H NMR (400 MHz, CDCl₃) δ=9.00 (d, 1H), 8.71 (d, 1H), 7.36-7.28 (m, 6H), 7.15 (dd, 4H), 5.31 (s, 1H), 4.73 (s, 4H); LC/MS m/z (M+H)⁺=364.1.

### Step 3: tert-butyl (6-(Dibenzylamino)-5-nitropyridin-3-yl)carbamate (25c)

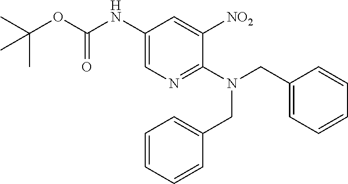 [SMILES: CC(C)(C)OC(=O)Nc1cnc(N(Cc2ccccc2)Cc2ccccc2)c([N+](=O)[O-])c1]

The following reaction was carried out in 16 batches in parallel. A solution of Preparation 25b (180 g, 495 mmol), t-BuOH (284 mL, 2.97 mol), Et₃N (207 mL, 1.49 mol) and diphenylphosphoryl azide (140 mL, 644 mmol) in toluene (1.8 L) was stirred over molecular sieves (4 A, 70 g) and this mixture was fed through a flow reactor (Reactor: 4 parallel Hastelloy coils (6 mm diameter, 6 m length); residence time: 200 min) at about 130° C. The 16 reaction batches were cooled to RT and combined with 2 additional identical reactions using 170 g and 50 g of Preparation 25b and similar proportions of other reagents. The combined reaction mixtures were washed with sat. aq. NaHCO₃ (3×5 L). The aqueous washes were combined and extracted with EtOAc (3×3 L). The combined EtOAc extracts were washed with brine (2×5 L), dried (Na₂SO₄), filtered and concentrated. The crude product was purified by chromatography (EtOAc/PE (w/0.5% v/v Et₃N)=0-30%) to provide the title compound (2.5 Kg, 66%). ¹H NMR (400 MHz, CDCl₃) δ=8.38 (br s, 1H), 8.32-8.26 (m, 1H), 7.31-7.21 (m, 6H), 7.19-7.13 (m, 4H), 6.48 (s, 1H), 4.52 (s, 4H), 1.52 (s, 9H); LC/MS m/z (M+H)⁺=435.2.

### Step 4: tert-Butyl (6-(dibenzylamino)-5-nitropyridin-3-yl)(methyl)carbamate (25d)

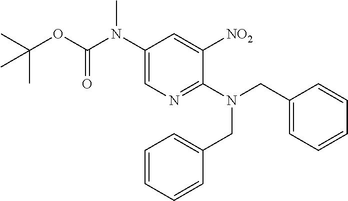 [SMILES: CN(C(=O)OC(C)(C)C)c1cnc(N(Cc2ccccc2)Cc2ccccc2)c([N+](=O)[O-])c1]

The following reaction was carried out in three parallel batches. A solution of Preparation 25c (400 g, 921 mmol) in THF (2.4 L) at about 0° C. was treated with KOtBu (113.6 g, 1.01 mol) in portions. The resulting mixture was stirred at about 0° C. for about 1 h. The mixture was treated with methyl iodide (68.7 mL, 1.10 mol) at about 0° C. The mixture was allowed to warm up to RT and stirred for about 16 h. The three batches were combined, filtered through Celite© and concentrated. The resulting residue was diluted with EtOAc (6 L) and washed with sat. aq. NH₄Cl (3×2 L). The aqueous washes were combined and extracted with EtOAc (2×1 L). The EtOAc extracts were combined, washed with brine (2×1 L), dried (Na₂SO₄), filtered, and concentrated. The resulting crude was purified by chromatography (silica, 0-20% EtOAc/PE (w/0.5% v/v Et₃N). The residue was triturated with PE (2 L) and stirred at RT for about 16 h. The resulting solids were collected by filtration and washed with PE (500 mL). The collected solids were dried under vacuum to provide the title compound (970 g, 77%). ¹H NMR (400 MHz, CDCl₃) δ=8.36 (br s, 1H), 8.05 (br s, 1H), 7.34-7.24 (m, 6H), 7.22-7.15 (m, 4H), 4.60 (s, 4H), 3.28 (s, 3H), 1.49 (s, 9H); LC/MS m/z (M+H)⁺=449.2.

### Step 5: tert-Butyl (5,6-diaminopyridin-3-yl)(methyl)carbamate

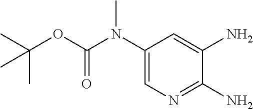 [SMILES: CN(C(=O)OC(C)(C)C)c1cnc(N)c(N)c1]

The following reaction was carried out in four parallel batches. A solution of Preparation 25d (103 g, 229 mmol) in ethanol (1 L) was treated with Pd(OH)₂/C (20.55 g) at RT. The suspension was stirred at about 80° C. under H₂ (50 PSI) for about 120 h. The reaction mixture was cooled to RT and filtered. The filter cake was washed with ethanol (200 mL×3). The filtrate was concentrated to a volume of approximately 600 mL. The solution was treated with Pd(OH)₂/C (20.5 g) and the resulting suspension was stirred at about 80° C. under H₂ (50 PSI) for about 48 h. The reaction mixture was cooled to RT. The four batches were combined with another two from identical reactions using 120 g and 360 g of tert-butyl (6-(dibenzylamino)-5-nitropyridin-3-yl)(methyl)carbamate, respectively, and similar proportions of other reagents. The resulting mixture was filtered and the filter cake was rinsed with ethanol (3×500 mL). The filtrate was concentrated and the resulting residue was purified by chromatography (silica, 0-10% DCM/methanol) to provide the title compound (245 g, 52%). ¹H NMR (400 MHz, CDCl₃) δ=7.55 (d, 1H), 6.84 (br s, 1H), 4.17 (br s, 2H), 3.33 (br s, 2H), 3.18 (s, 3H), 1.44 (br s, 9H); LC/MS m/z (M+H)⁺=239.2.

### Preparation 26

### N²,N²-Dibenzyl-3-nitropyridine-2,5-diamine

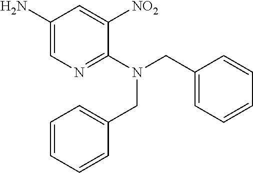 [SMILES: Nc1cnc(N(Cc2ccccc2)Cc2ccccc2)c([N+](=O)[O-])c1]

A solution of Preparation 25c (1.0 g, 2.3 mmol) in DCM (10 mL) was treated with TFA (2 mL) at about 30° C. and stirred for about 18 h. The solvent was evaporated and the residue was treated with sat. aq. NaHCO₃. The mixture was extracted with EtOAc (15 mL×3). The EtOAc extracts were combined, dried (NaSO₄), filtered and concentrated to provide the title compound (0.77 g). ¹H NMR (400 MHz, CDCl₃) δ=7.99 (d, 1H), 7.50 (d, 1H), 7.33-7.17 (m, 10H), 4.43 (s, 4H), 3.58 (br s, 2H).

### Preparation 27

### tert-Butyl methyl(2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-yl)carbamate

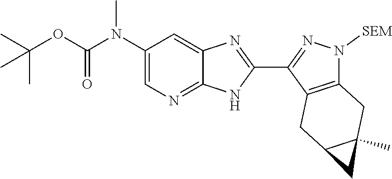 [SMILES: CN(C(=O)OC(C)(C)C)c1cnc2[nH]c(-c3nn(COCC[Si](C)(C)C)c4c3C[C@@H]3C[C@]3(C)C4)nc2c1]

A mixture of Preparation 25 (5.00 g, 20.1 mmol) and sodium metabisulfite (1.99 g, 10.5 mmol) in DMF (105 mL) was treated with Preparation 9 (6.43 g, 21.0 mmol) and DMSO (3.72 mL, 52.5 mmol). The reaction mixture was heated at about 110° C. for about 16 h. The reaction mixture was poured into a 3% aq. LiCl (250 mL). The resulting precipitate was collected by filtration and the filtrate extracted with EtOAc (3×200 mL). The EtOAc extracts were combined with the collected precipitate and concentrated. The crude product was purified by chromatography (silica; PE/EtOAc: 0-100% then EtOAc/ethanol: 0-20%) to provide the title compound (7.2 g, 65%). ¹H NMR (400 MHz, CDCl₃) δ=11.74-11.28 (m, 1H), 8.39 (br d, 1H), 7.93 (d, 1H), 5.50-5.34 (m, 2H), 3.58 (t, 2H), 3.34 (s, 3H), 3.24-3.10 (m, 2H), 2.76 (br d, 1H), 1.68 (s, 2H), 1.43 (br s, 9H), 1.31 (s, 3H), 1.21-1.13 (m, 1H), 0.97-0.84 (m, 2H), 0.44 (dd, 1H), 0.30 (br t, 1H), −0.03 (s, 9H); LC/MS m/z (M+H)⁺=525.3.

### Preparation 28

### N-Methyl-2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-amine

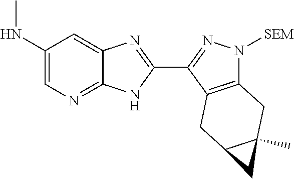

A solution of Preparation 27 (7.2 g, 13.7 mmol) in DCM (343 mL) was treated with ZnBr₂ (15.6 g, 68.6 mmol) at about 0° C. The reaction mixture was stirred at about 15° C. for about 16 h. The mixture was poured into a mixture of ice and sat. aq. NaHCO₃ (300 mL). The mixture was filtered and the filtrate was extracted with DCM (3×200 mL). The combined DCM extracts were concentrated. The resulting residue was purified via chromatography (silica, 15-100% EtOAc/PE then 20% ethanol/EtOAc) to provide the title compound (5.0 g, 86%). ¹H NMR (400 MHz, CD₃OD) δ=8.00 (br s, 1H), 7.15 (br s, 1H), 5.29 (br s, 2H), 3.43-3.27 (m, 2H), 3.13-2.94 (m, 2H), 2.80 (br s, 4H), 2.68-2.42 (m, 1H), 1.23 (s, 3H), 1.07-0.93 (m, 1H), 0.77-0.59 (m, 2H), 0.47-0.28 (m, 1H), 0.18-0.03 (m, 1H), −0.01-−0.26 (m, 9H); LC/MS m/z (M+H)⁺=425.3.

### Preparation 29

### (4aS,5aR)-3-(6-Bromo-1H-imidazo[4,5-b]pyridin-2-yl)-5a-methyl-1-((2-(trimethylsilyl)ethoxy)methyl)-1,4,4a,5,5a,6-hexahydrocyclopropa[f]indazole

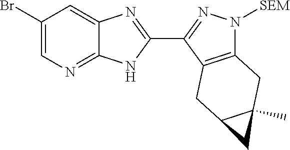 [SMILES: C[C@@]12Cc3c(c(-c4nc5cc(Br)cnc5[nH]4)nn3COCC[Si](C)(C)C)C[C@@H]1C2]

A mixture of 2,3-diamino-5-bromopyridine (3.07 g, 16.3 mmol) and sodium metabisulfite (1.55 g, 8.16 mmol) in DMF (82 mL) was treated with Preparation 9 (5.0 g, 16.3 mmol) and DMSO (2.90 mL, 40.8 mmol) at about 15° C. The resulting mixture was stirred at about 110° C. for about 16 h. The reaction mixture was combined with that from an identical reaction using 20 mg of 2,3-diamino-5-bromopyridine and similar proportions of other reagents. To the combined reaction mixtures was added H₂O (100 mL). The mixture was filtered and the precipitate was collected, triturated with 10% v/v EtOAc in PE (80 mL) and stirred at about 15° C. for about 2 h. The solids were filtered, washed with PE (50 mL) and dried to provide the title compound (6.65 g, 85%). ¹H NMR (400 MHz, CDCl₃) δ=12.92-12.83 (m, 1H), 8.66 (br s, 1H), 8.25 (br s, 1H), 5.51 (q, 2H), 3.66 (t, 2H), 3.57 (d, 1H), 3.24-3.11 (m, 2H), 2.78 (d, 1H), 1.32 (s, 3H), 1.23-1.13 (m, 1H), 0.94 (t, 2H), 0.49-0.41 (m, 1H), 0.34-0.27 (m, 1H), −0.05 (s, 9H); LC/MS m/z (M+H)⁺=476.1.

### Preparations 30a and 30b

### (4aS,5aR)-3-(6-Bromo-1-((2-(trimethylsilyl)ethoxy)methyl)-1H-imidazo[4,5-b]pyridin-2-yl)-5a-methyl-1-((2-(trimethylsilyl)ethoxy)methyl)-1,4,4a,5,5a,6-hexahydrocyclopropa[f]indazole (30a) and (4aS,5aR)-3-(6-bromo-3-((2-(trimethylsilyl)ethoxy)methyl)-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (30b)

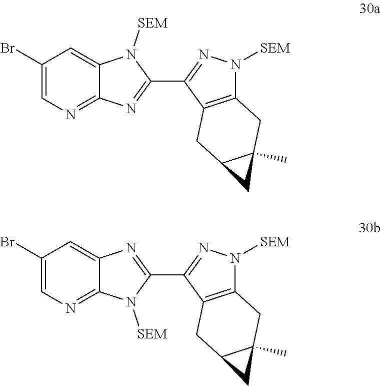 [SMILES: C[C@@]12Cc3c(c(-c4nc5ncc(Br)cc5n4COCC[Si](C)(C)C)nn3COCC[Si](C)(C)C)C[C@@H]1C2 ; C[C@@]12Cc3c(c(-c4nc5cc(Br)cnc5n4COCC[Si](C)(C)C)nn3COCC[Si](C)(C)C)C[C@@H]1C2]

A solution of Preparation 29 (20.8 g, 43.8 mmol) in THF (219 mL) at about 0° C. was treated with sodium hydride (60% suspension, 1.75 g, 43.8 mmol) added in portions. The resulting mixture was stirred for about 30 min. The mixture was treated with SEM-Cl (2.55 g, 15.3 mmol) at about 0° C. The reaction mixture was stirred at about 15° C. for about 3 h. The mixture was cooled to about 0° C. and treated with water (10 mL). The mixture was purified by chromatography (silica, EtOAc/PE 0-100%) to provide Preparation 30a (4.8 g, 18%) and a mixture of Preparations 30a and 30b. This mixture was subjected again to chromatography (silica gel, EtOAc/PE 0-100%) to provide additional Preparation 30a (6.0 g, 23%) and Preparation 30b (3.7 g, 14%).

Preparation 30a: Yield: 10.8 g (41%); ¹H NMR (400 MHz, CDCl₃) δ=8.58 (d, 1H), 8.02 (d, 1H), 6.26-6.11 (m, 2H), 5.51-5.31 (m, 2H), 3.68-3.47 (m, 5H), 3.25-3.05 (m, 2H), 2.74 (d, 1H), 1.29 (s, 3H), 1.19-1.07 (m, 1H), 0.95-0.82 (m, 4H), 0.41 (dd, 1H), 0.25 (t, 1H), −0.01 (s, 9H), −0.10 (s, 9H).

Preparation 30b: Yield: 3.7 g (14%); ¹H NMR (400 MHz, CDCl₃) δ=8.42 (d, 1H), 8.19 (d, 1H), 6.28-6.12 (m, 2H), 5.52-5.36 (m, 2H), 3.66-3.53 (m, 4H), 3.48 (br d, 1H), 3.18-3.09 (m, 2H), 2.75 (d, 1H), 1.29 (s, 3H), 1.14 (td, 1H), 0.99-0.81 (m, 4H), 0.42 (dd, 1H), 0.28 (t, 1H), −0.01 (s, 9H), −0.13 (s, 9H).

### Preparation 31

### 2-((4aS,5aR)-5a-Methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine

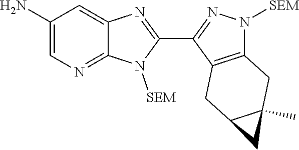 [SMILES: C[C@@]12Cc3c(c(-c4nc5cc(N)cnc5n4COCC[Si](C)(C)C)nn3COCC[Si](C)(C)C)C[C@@H]1C2]

A solution of Preparation 30b (0.72 g, 1.19 mmol) in NMP (8 mL) was treated with Cu₂O (0.17 g, 1.2 mmol) and conc. NH₄OH (8 mL) at RT. The resulting suspension was degassed with N_{2(g)} for about 2 min. The mixture was sealed in a microwave tube and irradiated in the microwave at 100° C. for about 10 h. The reaction mixture was partitioned between EtOAc (20 mL) and H₂O (20 mL) and the layers were separated. The aqueous layer was extracted with EtOAc (20 mL). The EtOAc extracts were combined, dried, and concentrated. The residue was purified by chromatography (silica, EtOAc/PE 0-100%) to provide the title compound (0.50 g, 77%). ¹H NMR (400 MHz, CDCl₃) δ=8.11-8.01 (m, 1H), 7.15 (d, 1H), 6.09 (q, 2H), 5.40 (q, 2H), 3.59-3.46 (m, 5H), 3.20-3.05 (m, 2H), 2.72 (d, 1H), 1.28 (s, 3H), 1.18-1.07 (m, 1H), 0.91 (t, 2H), 0.86-0.79 (m, 2H), 0.41-0.35 (m, 1H), 0.24 (t, 1H), −0.03 (s, 9H), −0.12 (s, 9H).

### Preparation 32

### tert-Butyl (2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-yl)carbamate

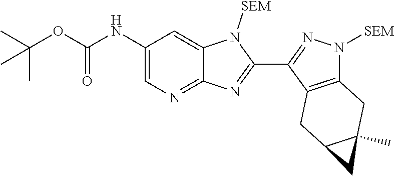 [SMILES: CC(C)(C)OC(=O)Nc1cnc2nc(-c3nn(COCC[Si](C)(C)C)c4c3C[C@@H]3C[C@]3(C)C4)n(COCC[Si](C)(C)C)c2c1]

A mixture of Preparation 30a (6.00 g, 9.92 mmol), t-butyl carbamate (0.77 g, 0.66 mmol), cesium carbonate (6.47 g, 19.8 mmol) in t-amyl alcohol (124 ml) under nitrogen was treated with Pd₂(dba)₃ (0.45 g, 0.50 mmol) and t-BuDavePhos (0.98 g, 1.98 mmol). The flask was sealed and the mixture was stirred at about 100° C. for about 48 h. Additional Pd₂(dba)₃ (0.23 g, 0.25 mmol) and t-BuDavePhos (340 mg, 0.99 mmol) were added and the mixture was stirred at about 100° C. for about 15 h. The mixture was cooled to RT and concentrated. The residue was purified by chromatography (silica, PE/EtOAc; 0-100%) to provide the title compound (2.76 g, 43%). ¹H NMR (400 MHz, CDCl₃) δ=8.47-8.33 (m, 1H), 8.21 (d, 1H), 6.71 (br s, 1H), 6.24-6.12 (m, 1H), 5.50-5.35 (m, 2H), 3.67-3.47 (m, 5H), 3.27-3.07 (m, 2H), 2.73 (br d, 1H), 1.55 (s, 9H), 1.28 (s, 3H), 1.16-1.11 (m, 1H), 0.96-0.82 (m, 4H), 0.40 (dd, 1H), 0.29-0.25 (m, 1H), −0.01 (s, 9H), −0.12 (s, 9H)

### Preparation 33

### tert-Butyl methyl(2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-yl)carbamate

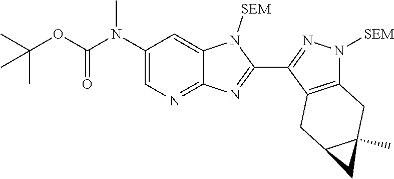 [SMILES: CN(C(=O)OC(C)(C)C)c1cnc2nc(-c3nn(COCC[Si](C)(C)C)c4c3C[C@@H]3C[C@]3(C)C4)n(COCC[Si](C)(C)C)c2c1]

A solution of Preparation 32 (3.16 g, 4.93 mmol) in THF (25 mL) at about 0° C. was treated with sodium hydride (60% suspension, 217 mg 5.42 mmol). The mixture was treated with methyl iodide (0.36 mL, 5.92 mmol) at about 0° C. and the reaction mixture was stirred at about 15° C. for about 15 h. The reaction mixture was quenched with sat. aq NH₄Cl (1 mL) and then with water (10 mL). The mixture was extracted with EtOAc (50 mL). The EtOAc layer was concentrated under reduced pressure. The residue was purified by chromatography (silica, PE/EtOAc 0-100%) to provide the title compound (3 g, 93%). ¹H NMR (400 MHz, CDCl₃) δ=8.43 (d, 1H), 7.74 (br s, 1H), 6.26-6.11 (m, 2H), 5.49-5.36 (m, 2H), 3.62-3.50 (m, 5H), 3.35 (s, 3H), 3.26-3.10 (m, 2H), 2.74 (br d, 1H), 1.44 (br s, 9H), 1.29 (s, 3H), 1.19-1.11 (m, 1H), 0.98-0.88 (m, 2H), 0.88-0.82 (m, 2H), 0.41 (dd, 1H), 0.29-0.16 (m, 1H), 0.06-−0.06 (m, 9H), −0.09-−0.18 (m, 9H).

### Preparation 34

### N-Methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine

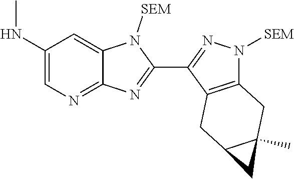 [SMILES: CNc1cnc2nc(-c3nn(COCC[Si](C)(C)C)c4c3C[C@@H]3C[C@]3(C)C4)n(COCC[Si](C)(C)C)c2c1]

A solution of Preparation 33 (2.90 g, 4.43 mmol) in DCM (50 mL) was treated with ZnBr₂ (4.99 g, 22.1 mmol) at about 0° C. The mixture was stirred at about 10° C. for about 16 h. The reaction mixture was poured into a mixture of ice and sat. aq. NaHCO₃ (10 mL). The mixture was extracted with DCM (3×100 mL). The combined DCM layers were dried (Na₂SO₄), filtered and concentrated. The crude product was purified by chromatography (silica, PE/EtOAc 0-70%) to provide the title compound (660 mg, 27%). LC/MS m/z (M+H)⁺=555.0.

### Preparation 35

### 2-(6,6-Dimethyl-1-((2-(trimethylsilyl)ethoxy)methyl)-4,5,6,7-tetrahydro-1H-indazol-3-yl)-N-methyl-3H-imidazo[4,5-b]pyridin-6-amine and 2-(6,6-dimethyl-2-((2-(trimethylsilyl)ethoxy)methyl)-4,5,6,7-tetrahydro-2H-indazol-3-yl)-N-methyl-3H-imidazo[4,5-b]pyridin-6-amine

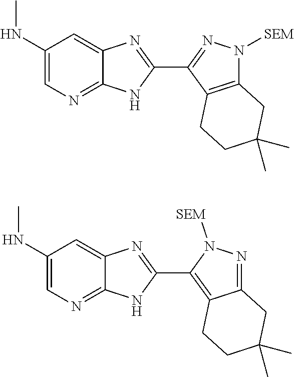 [SMILES: CNc1cnc2[nH]c(-c3nn(COCC[Si](C)(C)C)c4c3CCC(C)(C)C4)nc2c1 ; CNc1cnc2[nH]c(-c3c4c(nn3COCC[Si](C)(C)C)CC(C)(C)CC4)nc2c1]

### Step 1: tert-Butyl (2-(6,6-dimethyl-1-((2-(trimethylsilyl)ethoxy)methyl)-4,5,6,7-tetrahydro-1H-indazol-3-yl)-3H-imidazo[4,5-b]pyridin-6-yl)(methyl)carbamate and tert-butyl (2-(6,6-dimethyl-2-((2-(trimethylsilyl)ethoxy)methyl)-4,5,6,7-tetrahydro-2H-indazol-3-yl)-3H-imidazo[4,5-b]pyridin-6-yl)(methyl)carbamate (35a)

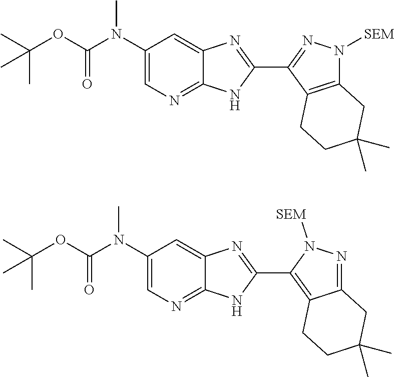

A solution of Preparation 19 (1.00 g, 3.24 mmol) in DMF (10 mL) and DMSO (2 mL) was treated with Preparation 25 (0.77 g, 3.24 mmol) and sodium metabisulfite (0.31 g, 1.62 mmol) at about 30° C. The reaction mixture was stirred at about 110° C. for about 18 h. Water (40 mL) was added and the precipitate collected by filtration, washed with water and dried under vacuum to provide the title compounds (1.7 g, 99%) as a mixture of regioisomers. LC/MS m/z (M+H)⁺=527.3.

### Step 2: 2-(6,6-Dimethyl-1-((2-(trimethylsilyl)ethoxy)methyl)-4,5,6,7-tetrahydro-1H-indazol-3-yl)-N-methyl-3H-imidazo[4,5-b]pyridin-6-amine and 2-(6,6-dimethyl-2-((2-(trimethylsilyl)ethoxy)methyl)-4,5,6,7-tetrahydro-2H-indazol-3-yl)-N-methyl-3H-imidazo[4,5-b]pyridin-6-amine

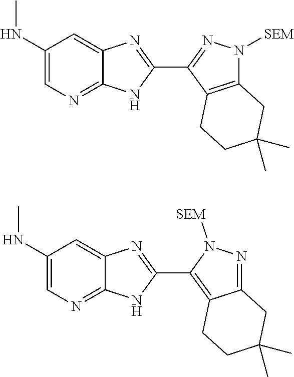 [SMILES: CNc1cnc2[nH]c(-c3nn(COCC[Si](C)(C)C)c4c3CCC(C)(C)C4)nc2c1 ; CNc1cnc2[nH]c(-c3c4c(nn3COCC[Si](C)(C)C)CC(C)(C)CC4)nc2c1]

A solution of Preparation 35a (1.0 g, 1.9 mmol) in DCM (25 mL) was treated with ZnBr₂ (2.14 g, 9.49 mmol) at about 30° C. The reaction mixture was stirred at about 30° C. for about 2 h. Water (25 mL) was added and the mixture was extracted into DCM (2×25 mL). The organic extracts were combined, dried (Na₂SO₄), filtered, and concentrated to provide the title compounds (0.81 g, 99%) as a mixture of regioisomers. LC/MS m/z (M+H)⁺=427.2.

### Preparation 36

### N-(5,6-Diaminopyridin-3-yl)-N-methylacetamide

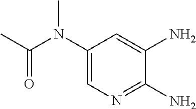

### Step 1: N-(6-(Dibenzylamino)-5-nitropyridin-3-yl)-N-methylacetamide (36a)

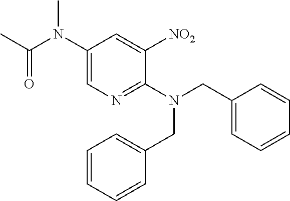 [SMILES: CC(=O)N(C)c1cnc(N(Cc2ccccc2)Cc2ccccc2)c([N+](=O)[O-])c1]

A solution of Preparation 26 (2.0 g, 5.74 mmol) in DCM (20 mL) at about 0° C. was treated with Et₃N (1.6 mL, 11.5 mmol), DMAP (70 mg, 0.57 mmol) and Ac₂O (0.65 mL, 6.89 mmol). The reaction mixture was stirred at about 15° C. for about 17 h. The reaction mixture was combined with that from an identical reaction using 100 mg of Preparation 26 and similar proportions of other reagents. The combined mixture was partitioned between sat. aq. NH₄Cl (20 mL) and DCM (20 mL). The aqueous layer was extracted with DCM (10 mL). The combined DCM extracts were dried (Na₂SO₄), filtered, and concentrated. The resulting residue was purified by chromatography (silica, EtOAc/PE=30-50%) to provide the title compound (2.4 g). ¹H NMR (400 MHz, CDCl₃) δ=8.28 (d, 1H), 7.96 (d, 1H), 7.35-7.28 (m, 6H), 7.19-7.14 (m, 4H), 4.65 (s, 4H), 3.26 (s, 3H), 1.92 (s, 3H).

### Step 2: N-(5,6-Diaminopyridin-3-yl)-N-methylacetamide

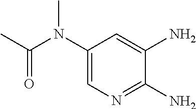

A solution of Preparation 36 (2.4 g, 6.15 mmol) in ethanol (30 mL) was treated with 10% Pd/C (300 mg) at about 10° C. The mixture was stirred at about 70° C. for about 18 h under 50 PSI of H₂. The reaction mixture was filtered and the filter cake was rinsed with ethanol. The filtrate was treated with 10% Pd/C (300 mg) and the mixture was stirred at about 70° C. for about 18 h under 50 PSI of H₂. The reaction mixture was filtered. The filtrate was concentrated to provide the title compound (1.1 g, 99%). ¹H NMR (400 MHz, CD₃OD) δ=7.27 (d, 1H), 6.73 (d, 1H), 3.38-3.25 (m, 3H), 1.87 (s, 3H).

### Preparation 37

### (S)-N-(5,6-Diaminopyridin-3-yl)-N-methyl-2-morpholinopropanamide

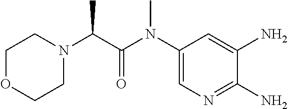 [SMILES: C[C@@H](C(=O)N(C)c1cnc(N)c(N)c1)N1CCOCC1]

### Step 1: N²,N²-Dibenzyl-N⁵-methyl-3-nitropyridine-2,5-diamine (37a)

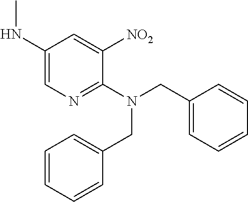 [SMILES: CNc1cnc(N(Cc2ccccc2)Cc2ccccc2)c([N+](=O)[O-])c1]

A solution of Preparation 25d (7.31 g, 16.3 mmol) in DCM (24.5 mL) was treated with TFA (16.4 mL, 8.12 mmol). The reaction mixture was stirred for about 2 h and concentrated. The resulting residue was triturated with toluene (2×) and heptane (2×). The residue was dissolved in 1:1 (v/v) THF/methanol (100 mL) and solid K₂CO₃ was added until pH 10 was achieved. The resulting mixture was stirred for about 30 min to 1 h and concentrated. EtOAc was added to the residue and the resulting mixture was washed with water and brine. The combined aqueous washes were extracted with EtOAc (2×), the organic extracts were combined, dried (MgSO₄), filtered, and concentrated. The resultant residue was triturated with diethyl ether (2×) to provide the title compound (5.3 g). LC/MS m/z (M+H)⁺=349.3.

### Step 2: (S)-N-(6-(Dibenzylamino)-5-nitropyridin-3-yl)-N-methyl-2-morpholinopropanamide (37b)

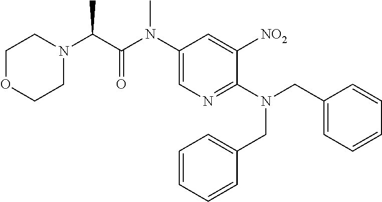 [SMILES: C[C@@H](C(=O)N(C)c1cnc(N(Cc2ccccc2)Cc2ccccc2)c([N+](=O)[O-])c1)N1CCOCC1]

A solution of Preparation 37a (5.3 g, 15.2 mmol) in pyridine (101 mL) was treated with EDCl (9.75 g, 45.6 mmol) and Preparation 20 (3.63 g, 22.8 mmol). The reaction mixture was stirred at RT for about 18 h. The mixture was treated with 0.1 N HCl and extracted with EtOAc (2×). The combined EtOAc extracts were washed with brine, dried (MgSO₄), filtered, and concentrated. The crude product was purified by chromatography (silica, 5-59% EtOAc/heptane) to provide the title compound (5.53 g, 74%). ¹H NMR (400 MHz, CD₃OD) δ=8.43 (d, 1H), 8.24 (d, 1H), 7.32-7.13 (m, 10H), 4.73 (q, 4H), 3.57-3.45 (m, 4H), 3.22 (s, 3H), 3.10 (q, 1H), 2.48-2.37 (m, 2H), 2.13-2.03 (m, 2H), 1.06 (d, 3H).

### Step 3: (S)-N-(5,6-Diaminopyridin-3-yl)-N-methyl-2-morpholinopropanamide

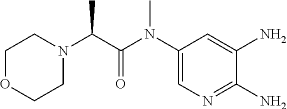 [SMILES: C[C@@H](C(=O)N(C)c1cnc(N)c(N)c1)N1CCOCC1]

A solution of Preparation 37b (7.80 g, 15.9 mmol) in ethanol (150 mL) was treated with 5% Pd/C (60% water content, 7.80 g). The mixture was stirred at about 70° C. under a H₂ atmosphere at 125 PSI for about 60 h. The reaction mixture was filtered through Celite© and the filter was washed with methanol and EtOAc. The filtrate was concentrated and the residue was triturated twice with diethyl ether. The crude product was purified by chromatography (silica, 0-16% methanol/DCM) to provide the title compound (3.65 g, 82%)¹H NMR (400 MHz, DMSO-d₆) δ=7.20 (d, 1H), 6.62 (d, 1H), 5.55 (br s, 2H), 4.89 (br s, 2H), 3.56-3.43 (m, 4H), 3.21 (q, 1H), 3.05 (s, 3H), 2.48-2.41 (m, 2H), 2.31-2.22 (m, 2H), 1.00 (d, 3H).

### Preparation 38

### (S)-N-(5,6-Diaminopyridin-3-yl)-N-ethyl-2-morpholinopropanamide

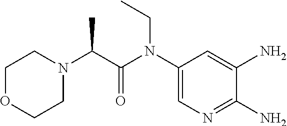 [SMILES: CCN(C(=O)[C@H](C)N1CCOCC1)c1cnc(N)c(N)c1]

### Step 1: tert-Butyl (6-(dibenzylamino)-5-nitropyridin-3-yl)(ethyl)carbamate (38a)

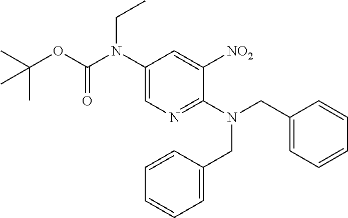 [SMILES: CCN(C(=O)OC(C)(C)C)c1cnc(N(Cc2ccccc2)Cc2ccccc2)c([N+](=O)[O-])c1]

A solution of Preparation 25c (1.0 g, 2.30 mmol) in THF (20 mL) was treated with sodium hydride (60% suspension, 0.14 g, 3.45 mmol) at about 0° C. for about 30 min. Ethyl iodide (0.37 mL, 4.60 mmol) was added dropwise at about 0° C. and the mixture was stirred at about 30° C. for about 60 h. Water (20 mL) was added and the mixture was extracted with EtOAc (3×50 mL). The combined EtOAc extracts were washed with brine and concentrated. The residue was purified by chromatography (silica, 0-10% EtOAc/PE) to provide the title compound (0.70 g, 66%). ¹H NMR (400 MHz, CDCl₃) δ=8.27 (br s, 1H), 7.97 (br s, 1H), 7.27 (s, 6H), 7.19-7.13 (m, 4H), 4.59 (s, 4H), 3.66 (q, 2H), 1.45 (br s, 9H), 1.18 (t, 3H)

### Step 2: N²,N²-dibenzyl-N5-ethyl-3-nitropyridine-2,5-diamine (38b)

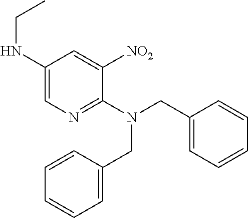 [SMILES: CCNc1cnc(N(Cc2ccccc2)Cc2ccccc2)c([N+](=O)[O-])c1]

A solution of Preparation 38a (0.70 g, 1.51 mmol) in DCM (5 mL) was treated with TFA (2 mL) at about 15° C. The reaction mixture was stirred at about 30° C. for about 18 h. The mixture was concentrated. The residue was dissolved in DCM (15 mL) and washed with sat. aq. NaHCO₃ (15 mL). The organic extracts were dried over Na₂SO₄ and concentrated to provide the title compound (0.60 g). LC/MS m/z (M+H)⁺=363.1.

### Step 3: (S)-N-(6-(Dibenzylamino)-5-nitropyridin-3-yl)-N-ethyl-2-morpholinopropanamide (38c)

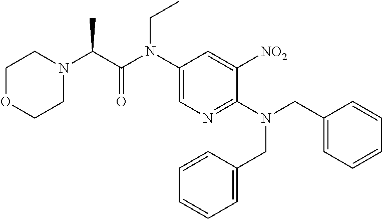 [SMILES: CCN(C(=O)[C@H](C)N1CCOCC1)c1cnc(N(Cc2ccccc2)Cc2ccccc2)c([N+](=O)[O-])c1]

A solution of Preparation 38b (0.35 g, 0.97 mmol) in pyridine (10 mL) was treated with EDCl (0.56 g, 2.90 mmol) and Preparation 20 (0.20 g, 1.26 mmol) at about 30° C. The reaction mixture was stirred for about 2h. Water (20 mL) was added and the mixture was extracted with EtOAc (2×20 mL). The combined EtOAc extracts were dried (Na₂SO₄), and concentrated. The residue was purified by chromatography (silica, 40-80% EtOAc/PE) to provide the title compound (0.40 g, 82%). LC/MS m/z (M+H)⁺=504.2.

### Step 4: (S)-N-(5,6-Diaminopyridin-3-yl)-N-ethyl-2-morpholinopropanamide

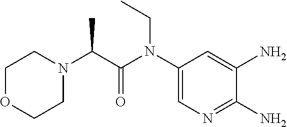 [SMILES: CCN(C(=O)[C@H](C)N1CCOCC1)c1cnc(N)c(N)c1]

A solution of Preparation 38c (0.40 g, 0.79 mmol) in ethanol (30 mL) was treated with Pd(OH)₂/C (112 mg) and 10% Pd/C (85 mg). The reaction mixture was stirred at about 70° C. for about 18 h under an atmosphere of H₂ at 50 PSI. The reaction mixture was filtered and the filtrate was concentrated to provide the title compound (233 mg). LC/MS m/z (M+H)⁺=294.0.

### Preparation 39

### (R)-N-(5,6-Diaminopyridin-3-yl)-N-ethyl-2-(tetrahydro-2H-pyran-4-yl)propenamide

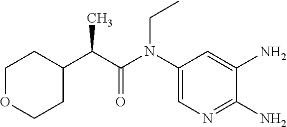 [SMILES: CCN(C(=O)[C@H](C)C1CCOCC1)c1cnc(N)c(N)c1]

### Step 1: (R)-N-(6-(Dibenzylamino)-5-nitropyridin-3-yl)-2-(tetrahydro-2H-pyran-4-yl)propenamide (39a)

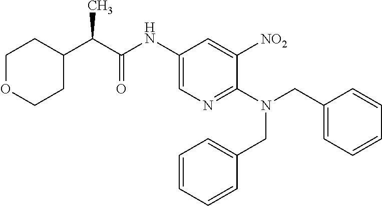 [SMILES: C[C@@H](C(=O)Nc1cnc(N(Cc2ccccc2)Cc2ccccc2)c([N+](=O)[O-])c1)C1CCOCC1]

A solution of Preparation 26 (740 mg, 2.21 mmol) in pyridine (10 mL) was treated with Preparation 21 (420 mg, 2.66 mmol) and EDCl (849 mg, 4.43 mmol) at about 30° C. and stirred for about 2 h. The reaction mixture was combined with that of another identical reaction conducted using 30 mg of Preparation 26 with similar proportions of other reagents. The combined reaction mixtures were diluted with water (20 mL) and extracted with EtOAc (2×20 mL). The combined EtOAc extracts were dried (Na₂SO₄), filtered and concentrated. The crude product was purified by chromatography (silica,.PE/EtOAc 10-30%) to provide the title compound (1.0 g, 92%). LC/MS m/z (M+H)⁺=475.2.

### Step 2: (R)-N²,N²-dibenzyl-N⁵-ethyl-3-nitro-N⁵-(1-(tetrahydro-2H-pyran-4-yl)ethyl)pyridine-2,5-diamine (39b)

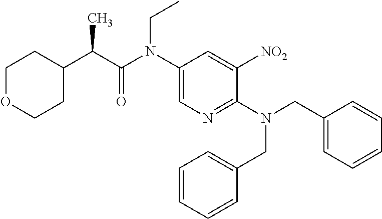 [SMILES: CCN(C(=O)[C@H](C)C1CCOCC1)c1cnc(N(Cc2ccccc2)Cc2ccccc2)c([N+](=O)[O-])c1]

A solution of Preparation 39a (0.70 g, 0.21 mmol) in DMF (20 mL) was treated with sodium hydride (60% suspension, 89 mg, 2.21 mmol) at about 30° C. The mixture was stirred at about 60° C. for about 1 h. The reaction mixture was cooled to about 30° C. and ethyl iodide (0.23 mL, 2.95 mmol) was added dropwise at about 30° C. The reaction mixture was stirred at about 60° C. for about 2 h. Water (35 mL) was added and the mixture was extracted with EtOAc (2×40 mL). The combined EtOAc extracts were dried (Na₂SO₄), filtered, and concentrated. The crude product was purified by chromatography to provide the title compound (0.65 g, 88%).LC/MS m/z (M+H)⁺=503.3.

### Step 3: (R)-N-(5,6-diaminopyridin-3-yl)-N-ethyl-2-(tetrahydro-2H-pyran-4-yl)propenamide

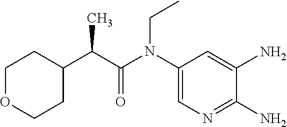 [SMILES: CCN(C(=O)[C@H](C)C1CCOCC1)c1cnc(N)c(N)c1]

A solution of Preparation 39b (650 mg, 1.29 mmol) in ethanol (30 mL) was treated with Pd(OH)₂/C (18 mg) and 10% Pd/C (138 mg). The reaction mixture was stirred under an atmosphere of H₂ at 50 PSI at about 70° C. for about 18 h. The reaction mixture was filtered and the filtrate was concentrated. The residue was dissolved in ethanol (30 mL) was treated with Pd(OH)₂/C (18 mg) and 10% Pd/C (138 mg). The reaction mixture was stirred under an atmosphere of H₂ at 50 PSI at about 70° C. for about 18. The reaction mixture was filtered and the filtrate was concentrated to provide the title compound (370 mg, 98%). LC/MS m/z (M+H)⁺=293.3.

### Preparation 40

### 2-((2-((4aS,5aR)-5a-Methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-yl)amino)ethan-1-ol

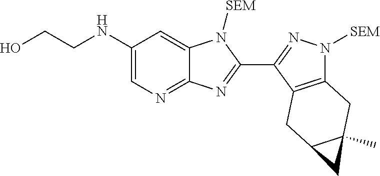 [SMILES: C[C@@]12Cc3c(c(-c4nc5ncc(NCCO)cc5n4COCC[Si](C)(C)C)nn3COCC[Si](C)(C)C)C[C@@H]1C2]

A solution of Preparation 30a (1.0 g, 1.65 mmol) in DMF (10 mL) was treated with N-(2,6-dimethylphenyl)-6-hydroxypicolinamide (0.16 g, 0.66 mmol) K₃PO₄ (1.05 g, 4.96 mmol), CuI (157 mg, 0.83 mmol) and 2-((tert-butyldimethylsilyl)oxy)ethan-1-amine (0.58 g, 3.31 mmol). The reaction mixture was stirred at about 110° C. for about 16 h. The mixture was diluted with EtOAc (20 mL), washed with water (10 mL), concentrated, and purified by chromatography to provide the title compound (430 mg, 88%). LC/MS m/z (M+H)⁺=585.4.

### Preparation 41

### 2-chloro-N-methyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-yl)propenamide

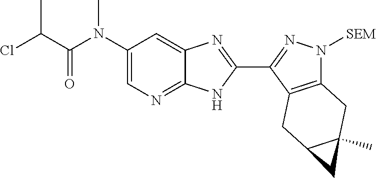 [SMILES: CC(Cl)C(=O)N(C)c1cnc2[nH]c(-c3nn(COCC[Si](C)(C)C)c4c3C[C@@H]3C[C@]3(C)C4)nc2c1]

A solution of Preparation 28 (5.5 g, 12.0 mmol) and 2-chloropropanoic acid (1.6 mL, 17.5 mmol) in pyridine (167 mL) was treated with EDCl (4.5 g, 23.3 mmol) at about 12° C. The reaction mixture was stirred at about 12° C. for about 3 h. The mixture was treated with water and extracted with EtOAc. The EtOAc extract was dried, filtered and concentrated to provide the title compound (6.30 g). ¹H NMR (400 MHz, CDCl₃) δ=12.29-12.01 (m, 1H), 8.87-8.75 (m, 1H), 8.47 (br s, 1H), 8.03 (br s, 1H), 7.93-7.78 (m, 1H), 7.46 (dd, 1H), 5.55-5.34 (m, 2H), 4.31 (br d, 1H), 3.72-3.49 (m, 3H), 3.40 (s, 3H), 3.23-3.07 (m, 2H), 2.78 (br d, 1H), 1.60 (d, 2H), 1.31 (s, 3H), 1.21-1.13 (m, 1H), 0.92 (br dd, 2H), 0.45 (br dd, 1H), 0.29 (br t, 1H), −0.03 (s, 9H).

### Preparation 42

### (5-Methoxy-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine and 5-methoxy-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine

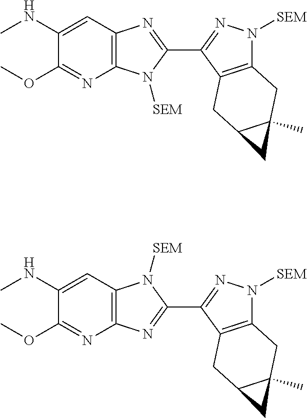

### Step 1: 6-methoxy-3-nitropyridin-2-amine (42a)

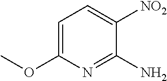

A suspension of 2-chloro-6-methoxy-3-nitropyridine (1.0 g, 5.20 mmol) in methanol (6 mL) was treated with 7M NH₃ in methanol (2.2 mL) and conc. NH₄OH (0.35 mL). The mixture was stirred at about 65° C. for about 18 h. The reaction mixture was cooled to RT and treated with water (5 mL). The solids were filtered and dried to provide the title compound. Yield: 762 mg (87%). ¹H NMR (400 MHz, CDCl₃) δ=8.30 (d, 1H), 6.15 (d, 1H), 3.94 (s, 3H).

### Step 2: 5-Bromo-6-methoxy-3-nitropyridin-2-amine (42b)

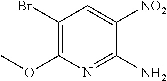

A suspension of Preparation 42a (762 mg, 4.51 mmol) and N-bromo succinimide (900 mg, 4.96 mmol) in AcOH (20 mL) was stirred at about 65° C. for about 1 h. The reaction mixture was poured into a mixture of sat. aq. NaHCO₃ and EtOAc. The aqueous phase was made basic with 1 N NaOH and extracted with EtOAc (2×). The combined organic extracts were dried (MgSO₄), filtered and concentrated. The crude product was purified by chromatography to provide the title compound. Yield: 988 mg (88%). ¹H NMR (400 MHz, CDCl₃) δ=8.54 (s, 1H), 4.02 (s, 3H).

### Step 3: 5-Bromo-6-methoxypyridine-2,3-diamine (42c)

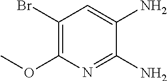

A suspension of Preparation 42b (500 mg, 2.02 mmol), NH₄Cl (1.2 g, 22.2 mmol) and zinc powder (1.32 g, 20.2 mmol) in ethanol (17 mL) and water (0.17 mL) was stirred at RT for about 18 h. The reaction mixture was filtered and the filtrate was concentrated. The residue was taken up in water and extracted with EtOAc (2×). The combined EtOAc extracts were dried (MgSO₄), filtered and concentrated to provide the title compound (440 mg). LC-MS m/z (M+H)⁺=218.0, 220.0 (⁷⁹Br, ⁸¹Br).

### Step 4: (4aS,5aR)-3-(6-Bromo-5-methoxy-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (42d)

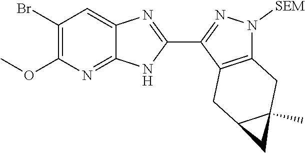

A suspension of Preparation 42c (440 mg, 2.02 mmol), Preparation 9 (618 mg, 2.02 mmol) and sodium metabisulfite (199 mg, 1.05 mmol) in DMF (12 mL) was treated with DMSO (0.36 mL, 5.04 mmol) and the mixture was stirred at about 110° C. for about 18 h. The mixture was cooled to RT, diluted with sat. aq. NaHCO₃ and extracted with DCM (3×). The combined DCM extracts were dried (MgSO₄), filtered and concentrated. The crude product was purified by chromatography to provide the title compound. Yield: 682 mg (67%). LC-MS m/z (M+H)⁺=504.3, 506.3 (⁷⁹Br, ⁸¹Br).

### Step 5: (4aS,5aR)-3-(6-Bromo-5-methoxy-1-((2-(trimethylsilyl)ethoxy)methyl)-1H-imidazo[4,5-b]pyridin-2-yl)-5a-methyl-1-((2-(trimethylsilyl)ethoxy)methyl)-1,4,4a,5,5a,6-hexahydrocyclopropa[f]indazole and (4aS,5aR)-3-(6-bromo-5-methoxy-3-((2-(trimethylsilyl)ethoxy)methyl)-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (42e)

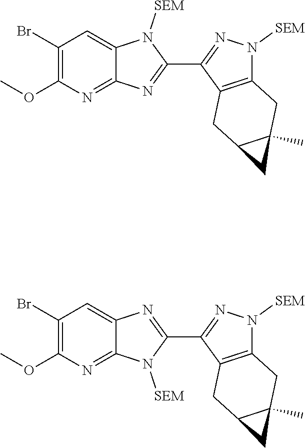

A solution of Preparation 42d (682 mg, 1.35 mmol) in THF (3.5 mL) was added dropwise to sodium hydride (60% suspension, 71 mg, 1.76 mmol) that had been washed with heptane (2×) and then suspended in THF (8 mL) at about 0° C. The mixture was stirred at about 0° C. for about 40 min. A solution of SEM-Cl (0.26 mL, 1.49 mmol) in THF (1.5 mL) was added dropwise and the mixture was stirred at about 0° C. for about 45 min and then at RT for about 2 h. The reaction mixture was treated with sat. aq. NH₄Cl and extracted with EtOAc (2×). The combined EtOAc extracts were dried (MgSO₄), filtered and concentrated. The crude product was purified by chromatography to provide the title compounds as a mixture of regioisomers. Yield: 731 mg (85%). LC-MS m/z (M+H)⁺=636.4.

### Step 6: (5-Methoxy-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine and 5-methoxy-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine (42f)

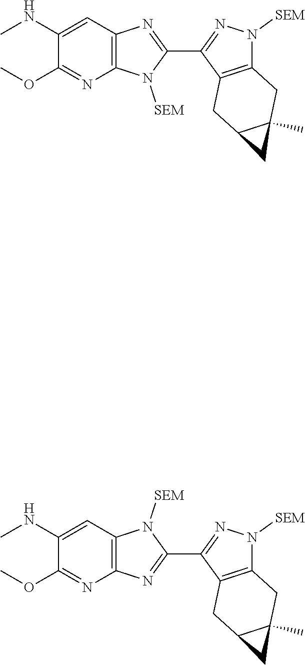

A suspension of Preparation 42e (380 mg, 0.60 mmol), CuI (17 mg, 0.09 mmol), K₃PO₄ (445 mg, 2.10 mmol) and N-(2,6-dimethylphenyl)-6-hydroxypicolinamide (44 mg, 0.18 mmol) in DMF (4.5 mL) under nitrogen was treated with methylamine hydrochloride (81 mg, 1.2 mmol). The mixture was stirred at about 110° C. for about 18 h. The mixture was cooled to RT and diluted with 1:4 v/v NH₄OH/water and extracted with EtOAc (2×). The combined EtOAc extracts were dried (MgSO₄), filtered and concentrated. The crude product was purified by chromatography to provide the title compounds as a mixture of regioisomers. Yield: 283 mg (85%). LC-MS m/z (M+H)⁺=585.5.

### Preparation 43

### 5-(2-Methoxyethoxy)-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine and 5-(2-methoxyethoxy)-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine

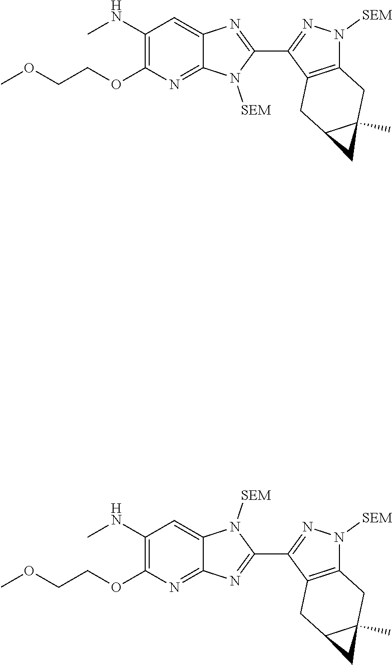

### Step 1: 5-Bromo-6-(2-methoxyethoxy)-3-nitropyridin-2-amine (43a)

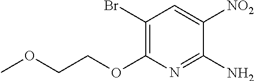 [SMILES: COCCOc1nc(N)c([N+](=O)[O-])cc1Br]

A solution of 2-methoxyethanol (0.29 mmol, 3.65 mmol) in THF (1 mL) was added dropwise to sodium hydride (60% suspension, 183 mg, 4.56 mmol) that had been washed with heptane (2×) and suspended in THF (12 mL) at about 0° C. The mixture was stirred at about 0° C. for about 30 min. A solution of 2-amino-5-bromo-6-chloro-3-nitropyridine (800 mg, 3.0 mmol) in THF (9 mL) was added dropwise and the mixture was stirred at about 0° C. for about 1 h and then at RT for about 1 h. The reaction mixture was treated with sat. aq. NH₄Cl and extracted with EtOAc (2×). The combined EtOAc extracts were dried (MgSO₄), filtered and concentrated. The crude product was combined with that from another identical reaction using 200 mg of 2-amino-5-bromo-6-chloro-3-nitropyridine and similar proportions of other reagents. The combined material was purified by chromatography to provide the title compound. Yield: 890 mg (77%). ¹H NMR (400 MHz, CDCl₃) δ=8.55 (s, 1H), 4.61-4.49 (m, 2H), 3.82-3.73 (m, 2H), 3.47 (s, 3H).

### Step 2: 5-bromo-6-(2-methoxyethoxy)pyridine-2,3-diamine (43b)

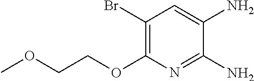

A suspension of Preparation 43a (500 mg, 1.71 mmol), NH₄Cl (1.01 g, 18.8 mmol) and zinc powder (1.12 g, 17.1 mmol) in ethanol (17 mL) and water (0.17 mL) was stirred at RT for about 18 h. The reaction mixture was filtered and the filtrate was concentrated. The residue was taken up in water and extracted with EtOAc (2×). The combined EtOAc extracts were dried (MgSO₄), filtered and concentrated to provide the title compound. Yield: 435 mg (97%), LC-MS m/z (M+H)⁺=264.1.

### Step 3: (4aS,5aR)-3-(6-bromo-5-(2-methoxyethoxy)-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (43c)

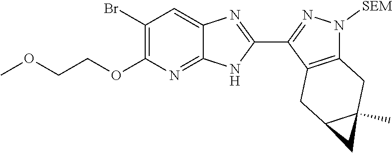

A suspension of Preparation 43b (435 mg, 1.66 mmol), Preparation 9 (509 mg, 1.66 mmol) and sodium metabisulfite (164 mg, 0.86 mmol) in DMF (12 mL) was treated with DMSO (0.29 mL, 4.15 mmol) and the mixture was stirred at about 110° C. for about 18 h. The mixture was cooled to RT, diluted with sat. aq. NaHCO₃ and extracted with DCM (3×). The combined DCM extracts were dried (MgSO₄), filtered and concentrated. The crude product was purified by chromatography to provide the title compound. Yield: 551 mg (61%). LC-MS m/z (M+H)⁺=550.2

### Step 4: (4aS,5aR)-3-(6-Bromo-5-(2-methoxyethoxy)-1-((2-(trimethylsilyl)ethoxy)methyl)-1H-imidazo[4,5-b]pyridin-2-yl)-5a-methyl-1-((2-(trimethylsilyl)ethoxy)methyl)-1,4,4a,5,5a,6-hexahydrocyclopropa[f]indazole and (4aS,5aR)-3-(6-bromo-5-(2-methoxyethoxy)-3-((2-(trimethylsilyl)ethoxy)methyl)-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (43d)

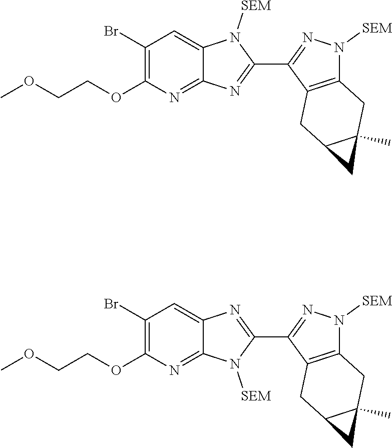

A solution of Preparation 43c (550 mg, 1.0 mmol) in THF (2 mL) was added dropwise to sodium hydride (60% suspension, 52 mg, 1.30 mmol) that had been washed with heptane (2×) and then suspended in THF (6 mL) at about 0° C. The mixture was stirred at about 0° C. for about 45 min. A solution of SEM-Cl (0.26 mL, 1.49 mmol) in THF (1 mL) was added dropwise and the mixture was stirred at RT for about 18 h. The reaction mixture was treated with sat. aq. NH₄Cl and extracted with EtOAc (2×). The combined EtOAc extracts were dried (MgSO₄), filtered and concentrated. The crude product was purified by chromatography to provide the title compounds as a mixture of regioisomers. Yield: 654 mg (96%). LC-MS m/z (M+H)⁺=680.3.

### Step 5: 5-(2-Methoxyethoxy)-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine and 5-(2-methoxyethoxy)-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine

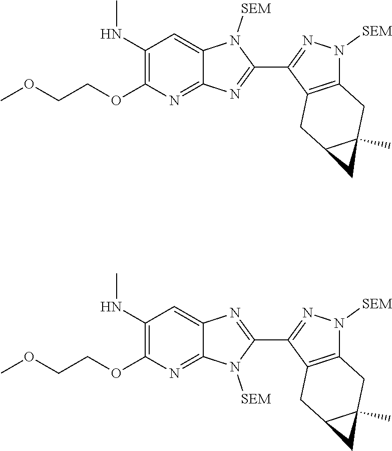

A suspension of Preparation 43d (654 mg, 0.96 mmol), CuI (27 mg, 0.15 mmol), K₃PO₄ (716 mg, 3.40 mmol) and N-(2,6-dimethylphenyl)-6-hydroxypicolinamide (70 mg, 0.29 mmol) in DMF (6 mL) under nitrogen and was treated with methylamine hydrochloride (130 mg, 1.93 mmol). The mixture was stirred at about 110° C. for about 18 h. The mixture was cooled to RT and diluted with 1:4 v/v NH₄OH/water and extracted with EtOAc (2×). The combined EtOAc extracts were dried (MgSO₄), filtered and concentrated. The crude product was purified by chromatography to provide the title compounds as a mixture of regioisomers. Yield: 461 mg (76%). LC-MS m/z (M+H)⁺=629.5.

### Preparation 44

### 5-Ethyl-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine and 5-ethyl-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine

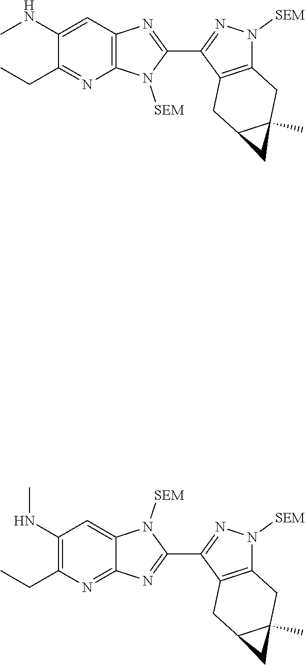

### Step 1: 5-Bromo-6-ethyl-3-nitropyridin-2-amine (44a)

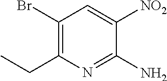

A suspension of 2-amino-6-ethyl-3-nitropyridine (868 mg, 5.19 mmol) and N-bromosuccinimide (1.04 g, 5.71 mmol) in AcOH (20 mL) was stirred at about 65° C. for about 1 h. The reaction mixture was poured into a biphasic mixture of sat. aq. NaHCO₃ and EtOAc. The mixture was treated with 1 N NaOH until basic and extracted with EtOAc (2×). The combined organic extracts were dried (MgSO₄), filtered and concentrated. The residue was combined with that from an identical reaction using 100 mg of 2-amino-6-ethyl-3-nitropyridine and with similar proportions of other reagents. The combined residues were purified by chromatography to provide the title compound. Yield: 1.04 mg (71%). ¹H NMR (400 MHz, CDCl₃) δ=8.52 (s, 1H), 2.88 (q, 2H), 1.27 (t, 3H).

### Step 2: 5-Bromo-6-ethylpyridine-2,3-diamine (44b)

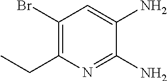

A suspension of Preparation 44a (500 mg, 2.03 mmol), NH₄Cl (1.2 g, 22.4 mmol) and zinc powder (1.33 g, 20.3 mmol) in ethanol (17 mL) and water (0.17 mL) was stirred at RT for about 18 h. The reaction mixture was filtered and the filtrate was concentrated. The residue was taken up in water and extracted with EtOAc (2×). The combined EtOAc extracts were dried (MgSO₄), filtered and concentrated to provide the title compound. Yield: 276 mg (63%), LC-MS m/z (M+H)⁺=216.1.

### Step 3: (4aS,5aR)-3-(6-Bromo-5-ethyl-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (44c)

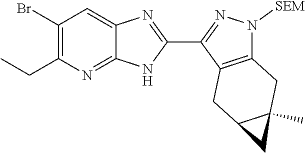 [SMILES: CCc1nc2[nH]c(-c3nn(COCC[Si](C)(C)C)c4c3C[C@@H]3C[C@]3(C)C4)nc2cc1Br]

A suspension of Preparation 44b (276 mg, 1.27 mmol), Preparation 9 (388 mg, 1.27 mmol) and sodium metabisulfite (125 mg, 0.66 mmol) in DMF (9 mL) was treated with DMSO (0.23 mL, 3.16 mmol) and the mixture was stirred at about 110° C. for about 18 h. The mixture was cooled to RT, diluted with sat. aq. NaHCO₃ and extracted with DCM (3×). The combined DCM extracts were dried (MgSO₄), filtered and concentrated. The crude product was purified by chromatography to provide the title compound. Yield: 401 mg (63%). LC-MS m/z (M+H)⁺=504.2.

### Step 4: (4aS,5aR)-3-(6-Bromo-5-ethyl-3-((2-(trimethylsilyl)ethoxy)methyl)-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole and (4aS,5aR)-3-(6-bromo-5-ethyl-1-((2-(trimethylsilyl)ethoxy)methyl)-1H-imidazo[4,5-b]pyridin-2-yl)-5a-methyl-1-((2-(trimethylsilyl)ethoxy)methyl)-1,4,4a,5,5a,6-hexahydrocyclopropa[f]indazole (44d)

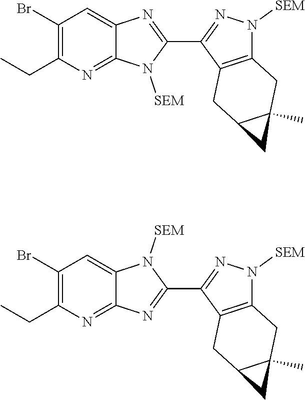

A solution of Preparation 44c (401 mg, 0.80 mmol) in THF (2 mL) was added dropwise to sodium hydride (60% suspension, 42 mg, 1.04 mmol) that had been washed with heptane (2×) and then suspended in THF (4 mL) at about 0° C. The mixture was stirred at about 0° C. for about 45 min. A solution of SEM-Cl (0.16 mL, 0.88 mmol) in THF (1 mL) was added dropwise and the mixture was stirred at RT for about 18 h. The reaction mixture was treated with sat. aq. NH₄Cl and extracted with EtOAc (2×). The combined EtOAc extracts were dried (MgSO₄), filtered and concentrated. The crude product was purified by chromatography to provide the title compounds as a mixture of regioisomers. Yield: 397 mg (79%). LC-MS m/z (M-CH₂CH₂TMS)⁺=532.3.

### Step 5: 5-Ethyl-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine and 5-ethyl-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-amine

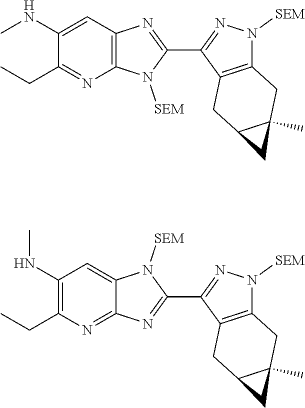

A suspension of Preparation 44d (397 mg, 0.63 mmol), CuI (17 mg, 0.09 mmol), K₃PO₄ (466 mg, 2.20 mmol) and N-(2,6-dimethylphenyl)-6-hydroxypicolinamide (46 mg, 0.19 mmol) in DMF (4.5 mL) under nitrogen and was treated with methylamine hydrochloride (85 mg, 1.25 mmol). The mixture was stirred at about 110° C. for about 18 h. The mixture was cooled to RT and diluted with 1:4 v/v NH₄OH/water and extracted with EtOAc (2×). The combined EtOAc extracts were dried (MgSO₄), filtered and concentrated. The crude product was purified by chromatography to provide the title compounds as a mixture of regioisomers. Yield: 93 mg (25%). LC-MS m/z (M+H)⁺=583.5.

### Preparation 45

### (S)-N-(5-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-N-methyl-2-morpholinopropanamide

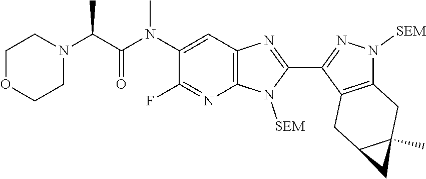 [SMILES: C[C@@H](C(=O)N(C)c1cc2nc(-c3nn(COCC[Si](C)(C)C)c4c3C[C@@H]3C[C@]3(C)C4)n(COCC[Si](C)(C)C)c2nc1F)N1CCOCC1]

### Step 1: 5-Bromo-6-chloro-3-nitropyridin-2-amine (45a)

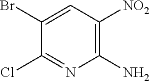 [SMILES: Nc1nc(Cl)c(Br)cc1[N+](=O)[O-]]

A suspension of 6-chloro-3-nitropyridin-2-amine (10.0 g, 57.6 mmol) in AcOH (200 mL) was treated with N-bromo succinimide (11.3 g, 63.4 mmol) at RT. The reaction mixture was stirred at about 60° C. for about 16 h. The precipitate was collected by filtration and washed with water (2×100 mL) and then dried under vacuum to provide the title compound (13.4 g, 92%). ¹H NMR (400 MHz, DMSO-d₆) δ=8.63 (s, 1H), 8.31 (br s, 2H).

### Step 2: tert-Butyl (5-bromo-6-chloro-3-nitropyridin-2-yl)(tert-butoxycarbonyl)carbamate (45b)

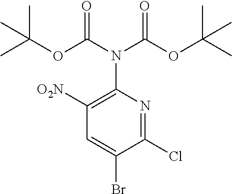 [SMILES: CC(C)(C)OC(=O)N(C(=O)OC(C)(C)C)c1nc(Cl)c(Br)cc1[N+](=O)[O-]]

A solution of Preparation 45a (11.2 g, 44.4 mmol) in THF (222 mL) was treated with DMAP (542 mg, 4.44 mmol) and BOC₂O (19.4 g, 88.7 mmol) at RT. The reaction mixture was stirred at about 70° C. for about 16 h. The reaction was cooled to RT, treated with water (100 mL) and extracted with EtOAc (3×100 mL). The combined EtOAc extracts were concentrated and the residue was purified by chromatography (silica, EtOAc/PE=0-10%) to provide the title compound (17 g, 85%). ¹H NMR (400 MHz, CDCl₃) δ=8.67 (s, 1H), 1.45 (s, 18H).

### Step 3: tert-Butyl (5-Bromo-6-fluoro-3-nitropyridin-2-yl)(tert-butoxycarbonyl)carbamate (45c)

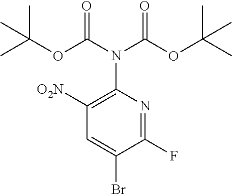 [SMILES: CC(C)(C)OC(=O)N(C(=O)OC(C)(C)C)c1nc(F)c(Br)cc1[N+](=O)[O-]]

A solution of Preparation 45b (15.2 g, 33.6 mmol) in DMSO (168 mL) was treated with potassium fluoride (9.75 g, 168 mmol). The mixture was flushed with nitrogen for about 3 min and stirred at RT for about 16 h. The reaction mixture was treated with 3% aq. LiCl (100 mL) and extracted with EtOAc (3×100 mL). The combined EtOAc extracts were concentrated and the residue was purified by chromatography (silica, EtOAc/PE=0-5%) to provide the title compound (9.9 g, 68%). ¹H NMR (400 MHz, CDCl₃) δ=8.76 (d, 1H), 1.45 (s, 18H).

### Step 4: 5-Bromo-6-fluoro-3-nitropyridin-2-amine hydrochloride (45d)

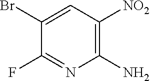 [SMILES: Nc1nc(F)c(Br)cc1[N+](=O)[O-]]

A solution of Preparation 45c (8.60 g, 19.71 mmol) in DCM (60 mL) at about 0° C. was treated with 4 N HCl in 1,4-dioxane (60 mL) dropwise. The reaction mixture was stirred at RT for about 16 h. The reaction mixture was concentrated to provide the title compound (4.7 g, 87%). ¹H NMR (400 MHz, DMSO-d₆) δ=8.49 (d, 1H), 8.36 (br s, 3H).

### Step 5: 5-Bromo-6-fluoropyridine-2,3-diamine (45e)

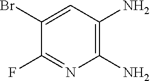

A solution of Preparation 45d (2.70 g, 9.91 mmol) in ethanol (50 mL) and water (50 mL) was treated with NH₄Cl (2.65 g, 49.5 mmol) and iron powder (2.77 g, 49.5 mmol) at RT. The reaction mixture was stirred at RT for about 16 h. The reaction mixture was filtered through Celite© and the filtrate was concentrated to remove most of the ethanol. The remaining mixture was extracted with EtOAc (3×50 mL). The combined EtOAc extracts were concentrated and the residue was purified by chromatography (silica, EtOAc/PE=20-80%). The isolated product was combined with that from an identical reaction using 1.4 g of Preparation 45d and similar proportions of other reagents to provide the title compound (2.3 g, 74%). ¹H NMR (400 MHz, DMSO-d₆) δ=6.93 (d, 1H), 6.01 (br s, 2H), 4.77 (br s, 2H).

### Step 6: (4aS,5aR)-3-(6-Bromo-5-fluoro-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (45f)

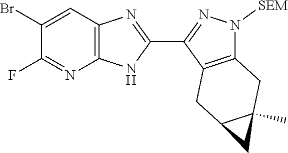 [SMILES: C[C@@]12Cc3c(c(-c4nc5cc(Br)c(F)nc5[nH]4)nn3COCC[Si](C)(C)C)C[C@@H]1C2]

A solution of Preparation 45e (720 mg, 3.49 mmol) and sodium metabisulfite (332 mg, 1.75 mmol) in DMF (20 mL) was treated with Preparation 9 (1.07 g, 3.49 mmol) and DMSO (0.62 mL, 8.74 mmol). The reaction mixture was stirred at about 110° C. for about 16 h. The reaction mixture was then stirred at about 130° C. for about 16 h. The reaction mixture was treated with 3% aq. LiCl (50 mL) and extracted with EtOAc (2×50 mL). The combined EtOAc extracts were concentrated and the residue was purified by chromatography (silica, EtOAc/PE=0-30%) to provide the title compound (1.4 g, 81%). ¹H NMR (400 MHz, CD₃OD) δ=8.43-8.10 (m, 1H), 5.58-5.40 (m, 2H), 3.62 (t, 2H), 3.42 (br d, 1H), 3.24-3.16 (m, 1H), 3.11 (dd, 1H), 2.77 (d, 1H), 1.31 (s, 3H), 1.22-1.14 (m, 1H), 0.89 (dd, 2H), 0.45 (dd, 1H), 0.25 (t, 1H), −0.03 (s, 9H).

### Step 7: (4aS,5aR)-3-(6-Bromo-5-fluoro-3-((2-(trimethylsilyl)ethoxy)methyl)-3H-imidazo[4,5-b]pyridin-2-yl)-5a-methyl-1-((2-(trimethylsilyl)ethoxy)methyl)-1.4.4a,5.5a,6-hexahydrocyclopropa[f]indazole (45q)

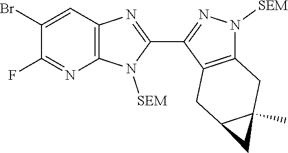 [SMILES: C[C@@]12Cc3c(c(-c4nc5cc(Br)c(F)nc5n4COCC[Si](C)(C)C)nn3COCC[Si](C)(C)C)C[C@@H]1C2]

A solution of Preparation 45f (1.40 g, 2.84 mmol) in THF (24 mL) at about 0° C. was treated with sodium hydride (60% suspension, 171 mg, 4.26 mmol) added in portions. The reaction mixture was stirred at about 0° C. for about 1 h. The reaction mixture was treated with SEM-Cl (0.50 mL, 2.84 mmol) dropwise. The reaction mixture was stirred at about 0° C. for about 1 h. The reaction mixture was treated with sat. aq. NH₄Cl (20 mL) at about 5° C. The mixture was extracted with EtOAc (2×20 mL). The combined EtOAc extracts were concentrated and the residue was purified by chromatography (silica, EtOAc/PE=0-30%) to provide the title compound (830 mg, 47%). ¹H NMR (400 MHz, CDCl₃) δ=8.30 (d, 1H), 6.24-6.00 (m, 2H), 5.52-5.36 (m, 2H), 3.61-3.51 (m, 4H), 3.46 (br d, 1H), 3.24-3.04 (m, 2H), 2.75 (br d, 1H), 1.29 (s, 3H), 1.18-1.09 (m, 1H), 1.02-0.80 (m, 4H), 0.42 (dd, 1H), 0.28 (t, 1H), −0.01 (s, 9H), −0.08-−0.19 (m, 9H).

### Step 8: tert-Butyl (5-fluoro-2-((4aS,5aR)-5a-methyl-1-((2-(trimethylsilyl)ethoxy)methyl)-1.4.4a,5.5a,6-hexahydrocyclopropa[f]indazol-3-yl)-3-((2-(trimethylsilyl)ethoxy)methyl)-3H-imidazo[4,5-b]pyridin-6-yl)carbamate (45h)

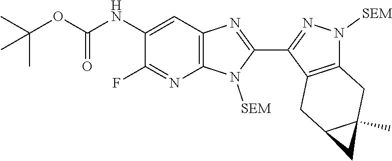 [SMILES: CC(C)(C)OC(=O)Nc1cc2nc(-c3nn(COCC[Si](C)(C)C)c4c3C[C@@H]3C[C@]3(C)C4)n(COCC[Si](C)(C)C)c2nc1F]

A suspension of Preparation 45 g (830 mg, 1.33 mmol), t-butyl carbamate (187 mg, 1.60 mmol) and cesium carbonate (869 mg, 2.67 mmol) in t-amyl alcohol (13 mL) under nitrogen was treated with treated with Pd₂(dba)₃ (122 mg, 0.13 mmol) and t-BuDavePhos (91 mg, 0.27 mmol). The mixture was sealed and stirred at about 100° C. for about 20 h. Additional t-butyl carbamate (156 mg, 1.33 mmol), Pd₂(dba)₃ (122 mg, 0.13 mmol) and t-BuDavePhos (91 mg, 0.27 mmol) were added and the reaction mixture stirred at about 100° C. for about 20 h. The reaction mixture was cooled to RT and concentrated. The residue was purified by chromatography (silica, PE/EtOAc 0-30%) to provide the title compound (220 mg, 25%). ¹H NMR (400 MHz, CDCl₃) δ=8.93-8.68 (m, 1H), 6.60 (br s, 1H), 6.22-6.00 (m, 2H), 5.47-5.36 (m, 2H), 3.64-3.52 (m, 4H), 3.45 (br d, 1H), 3.21-3.04 (m, 2H), 2.73 (br d, 1H), 2.59-2.49 (m, 1H), 1.55 (s, 9H), 1.29 (s, 3H), 1.20-1.10 (m, 1H), 0.99-0.80 (m, 4H), 0.41 (dd, 1H), 0.28 (t, 1H), −0.01 (s, 9H), −0.12 (s, 9H).

### Step 9: 5-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-amine (45i)

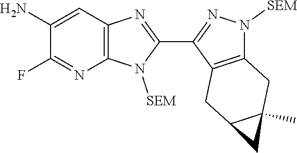 [SMILES: C[C@@]12Cc3c(c(-c4nc5cc(N)c(F)nc5n4COCC[Si](C)(C)C)nn3COCC[Si](C)(C)C)C[C@@H]1C2]

A solution of Preparation 45h (110 mg, 0.17 mmol) in DCM (5 mL) at about 5° C. was treated with ZnBr₂ (188 mg, 0.84 mmol). The reaction mixture was stirred at RT for about 18 h. The reaction mixture was poured into sat. aq. NaHCO₃ (20 mL) and extracted with DCM (2×20 mL). The combined DCM extracts were dried (MgSO₄), filtered and concentrated to provide the title compound (85 mg, 91%). LC-MS m/z (M+H)⁺=559.3.

### Step 10: (S)-N-(5-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide (45j)

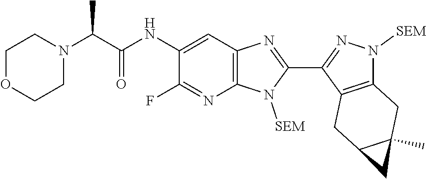 [SMILES: C[C@@H](C(=O)Nc1cc2nc(-c3nn(COCC[Si](C)(C)C)c4c3C[C@@H]3C[C@]3(C)C4)n(COCC[Si](C)(C)C)c2nc1F)N1CCOCC1]

A solution of Preparation 45i (85 mg, 0.15 mmol) and Preparation 20 (29 mg, 0.18 mmol) in pyridine (4 mL) was treated with EDCl (58 mg, 0.30 mmol) at RT. The reaction mixture was stirred at RT for about 16 h. Water (10 mL) was added to the reaction mixture and the mixture was extracted with EtOAc (2×10 mL). The combined EtOAc extracts were concentrated and the residue was purified by chromatography (silica, PE/EtOAc 0-50%) to provide the title compound (60 mg, 56%). LC-MS m/z (M+H)⁺=700.3.

### Step 11: (S)-N-(5-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-N-methyl-2-morpholinopropanamide

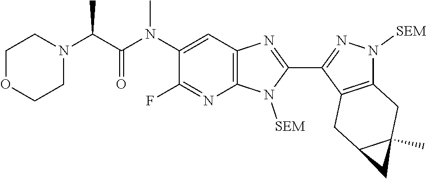 [SMILES: C[C@@H](C(=O)N(C)c1cc2nc(-c3nn(COCC[Si](C)(C)C)c4c3C[C@@H]3C[C@]3(C)C4)n(COCC[Si](C)(C)C)c2nc1F)N1CCOCC1]

A solution of Preparation 45j (60 mg, 0.09 mmol) in THF (5 mL) at about 0° C. was treated with 1 M KOtBu in THF (95 μL). The mixture was stirred at about 0° C. for about 1 h. The mixture was treated with methyl iodide (6 μL, 0.09 mmol) in THF (0.5 mL). The mixture was stirred at about 0° C. for about 1.5 h. Additional 1 M KOtBu in THF (25 μL) and methyl iodide (3 μL, 0.05 mmol) were added at about 5° C. and the mixture was stirred at RT for about 16 h. Additional 1 M KOtBu in THF (25 μL) and methyl iodide (3 μL, 0.05 mmol) were added at about 5° C. and the mixture was stirred at RT for about 1 h. The reaction mixture was treated with sat. aq. NH₄Cl (5 mL) and extracted with EtOAc (2×5 mL). The combined organic extracts were concentrated to provide the title compound (60 mg, 98%). LC-MS m/z (M+H)⁺=714.5.

### Preparation 46

### N,5-Dimethyl-2-((4aS,5aR)-5a-methyl-1,4,4a,5,5a,6-hexahydrocyclopropa[f]indazol-3-yl)-3H-imidazo[4,5-b]pyridin-6-amine

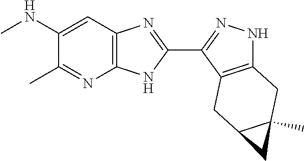

### Step 1: 5-Bromo-N-(4-methoxybenzyl)-6-methyl-3-nitropyridin-2-amine (46a)

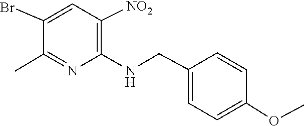 [SMILES: COc1ccc(CNc2nc(C)c(Br)cc2[N+](=O)[O-])cc1]

A suspension of 3-bromo-6-chloro-2-methyl-5-nitropyridine (4.19 g, 16.66 mmol) and potassium carbonate (5.76 g, 41.7 mmol) in THF (40 mL) at RT was treated dropwise with 4-methoxybenzyl amine (3.27 mL, 25.0 mmol) dissolved in THF (15 mL). The mixture was stirred at RT for about 30 min. The reaction mixture was treated with water and extracted with EtOAc (2×). The combined EtOAc extracts were dried (Na₂SO₄), filtered and concentrated to provide the title compound (5.87 g). 1H NMR (400 MHz, CDCl₃) δ=8.51 (s, 1H), 8.45-8.38 (m, 1H), 7.30 (d, 2H), 6.89 (d, 2H), 4.76 (d, 2H), 3.81 (s, 3H), 2.61 (s, 3H).

### Step 2: 5-Bromo-N²-(4-methoxybenzyl)-6-methylpyridine-2,3-diamine (46b)

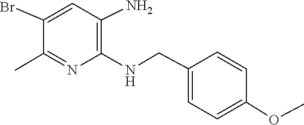 [SMILES: COc1ccc(CNc2nc(C)c(Br)cc2N)cc1]

A suspension of Preparation 46a (5.87 g, 16.7 mmol) and NH₄Cl (9.84 g, 184 mmol) in ethanol (56 mL) was treated with water (30 mL) and zinc powder (10.9 g, 167 mmol) at RT. The reaction mixture was stirred at RT for about 2 h. The reaction mixture was filtered through Celite© and the filter was washed with EtOAc and water. The filtrate was extracted with EtOAc. The combined EtOAc extracts were washed with brine, dried (Na₂SO₄), filtered and concentrated. The residue was triturated with diethyl ether and the undissolved material was filtered. The filtrate was concentrated and the residue was purified by chromatography (silica, EtOAc/heptane=0-100%) to provide the title compound (3.10 g, 57%). 1H NMR (400 MHz, CDCl₃) δ=7.33 (d, 2H), 6.98 (s, 1H), 6.88 (d, 2H), 4.54 (br s, 2H), 4.38 (br s, 1H), 3.81 (s, 3H), 3.13 (br s, 2H), 2.48 (s, 3H).

### Step 3: (4aS,5aR)-3-(6-Bromo-3-(4-methoxybenzyl)-5-methyl-3H-imidazo[4,5-b]pyridin-2-yl)-5a-methyl-1-((2-(trimethylsilyl)ethoxy)methyl)-1,4,4a,5,5a,6-hexahydrocyclopropa[f]indazole (46c)

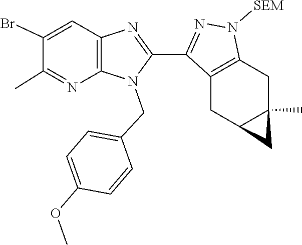 [SMILES: COc1ccc(Cn2c(-c3nn(COCC[Si](C)(C)C)c4c3C[C@@H]3C[C@]3(C)C4)nc3cc(Br)c(C)nc32)cc1]

A solution of Preparation 46b (500 mg, 1.55 mmol) and Preparation 9 (476 mg, 1.55 mmol) in DMF was treated with DMSO (0.28 mL, 3.88 mmol) and sodium metabisulfite (148 mg, 0.78 mmol). The reaction mixture was stirred at about 100° C. for about 18 h. The reaction mixture was cooled to RT and treated with sat. aq. NaHCO₃ and extracted with EtOAc. The EtOAc extract was washed with water (3×), dried (Na₂SO₄), filtered and concentrated. The residue was triturated with MeCN and the solids were filtered, washed with MeCN and dried to provide the title compound (359 mg, 38%). ¹H NMR (400 MHz, CDCl₃) δ=8.38-8.19 (m, 1H), 7.30-7.24 (m, 7H), 6.73 (d, 2H), 5.95 (s, 2H), 5.50-5.35 (m, 2H), 3.73 (s, 3H), 3.53 (t, 2H), 3.43 (br d, 1H), 3.13 (br d, 1H), 2.78 (s, 3H), 2.74 (br d, 1H), 1.29 (s, 3H), 1.18-1.08 (m, 1H), 0.94-0.86 (m, 2H), 0.40 (br dd, 1H), 0.23 (br t, 1H), −0.04 (s, 9H)

### Step 4: 3-(4-Methoxybenzyl)-N,5-dimethyl-2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-amine (46d)

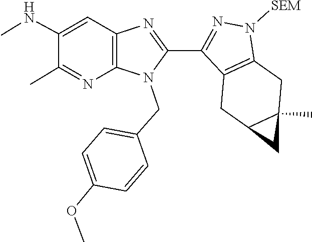

A suspension of Preparation 46c (357 mg, 0.59 mmol), methyl amine hydrochloride (59 mg, 0.88 mmol), N-(2,6-dimethylphenyl)-6-hydroxypicolinamide (14 mg, 0.06 mmol), K₃PO₄ (374 mg, 1.76 mmol) and CuI (11 mg, 0.06 mmol) in DMF (3 mL) was stirred under nitrogen at about 110° C. for about 18 h. The reaction mixture was cooled to RT, diluted with EtOAc and washed with water (3×). The EtOAc extract was dried (Na₂SO₄), filtered and concentrated. The crude product was purified by chromatography (silica, EtOAc/heptane=0-100%) to provide the title compound (53 mg, 16%). 1H NMR (400 MHz, CDCl₃) δ=7.38-7.29 (m, 1H), 7.24 (br d, 2H), 6.72 (br d, 2H), 5.92 (s, 1H), 5.52-5.34 (m, 2H), 3.72 (s, 3H), 3.53 (br t, 2H), 3.18-3.09 (m, 1H), 2.96 (s, 3H), 2.73 (br d, 1H), 2.51 (s, 3H), 1.57 (br s, 2H), 1.28 (s, 3H), 1.19-1.06 (m, 1H), 0.98-0.84 (m, 2H), 0.38 (br dd, 1H), 0.24 (br dd, 1H), −0.04 (s, 9H).

### Step 5: N,5-Dimethyl-2-((4aS,5aR)-5a-methyl-1,4,4a,5,5a,6-hexahydrocyclopropa[f]indazol-3-yl)-3H-imidazo[4,5-b]pyridin-6-amine

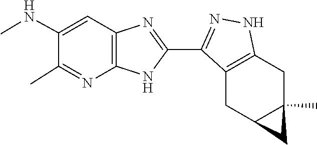

A suspension of Preparation 46d (53 mg, 0.095 mmol) in TFA (1 mL) was heated at about 75° C. for about 1 h. The reaction mixture was cooled to RT, diluted with water and made basic with 3 N NaOH. The mixture was extracted with a mixture of EtOAc/methanol. The organic extract was washed with brine, dried (Na₂SO₄), filtered and concentrated to provide the title compound (29 mg, 99%). LC-MS m/z (M+H)⁺=309.2.

### Preparation 47

### (S)-N-(2-((tert-Butyldimethylsilyl)oxy)ethyl)-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide

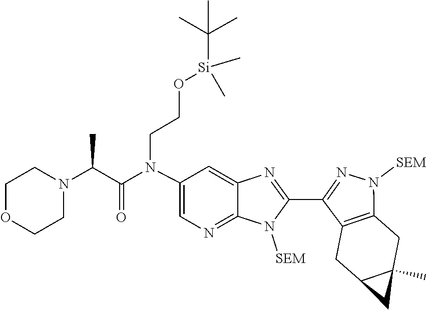 [SMILES: C[C@@H](C(=O)N(CCO[Si](C)(C)C(C)(C)C)c1cnc2c(c1)nc(-c1nn(COCC[Si](C)(C)C)c3c1C[C@@H]1C[C@]1(C)C3)n2COCC[Si](C)(C)C)N1CCOCC1]

### Step 1: (S)-N-(2-((4aS,5aR)-5a-Methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide (47a)

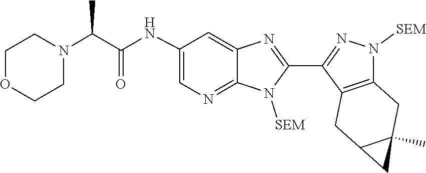

A solution of Preparation 31 (150 mg, 0.28 mmol) in pyridine (3 mL) was treated with EDCl (0.11 g, 0.56 mmol) and Preparation 20 (66 mg, 0.42 mmol). The reaction mixture was stirred at RT for about 16 h. The mixture was diluted with EtOAc (15 mL) and washed with water (15 mL). The organic extract was dried, filtered and concentrated. The residue was purified by chromatography to provide the title compound (120 mg, 63%). LC/MS m/z (M+H)⁺=682.3.

### Step 2: (S)-N-(2-((tert-butyldimethylsilyl)oxy)ethyl)-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide

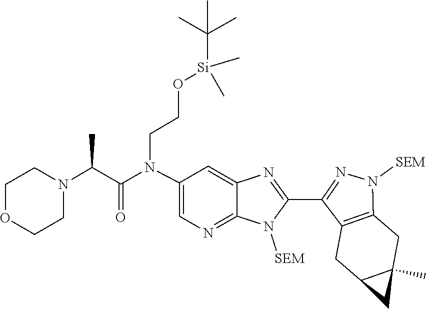 [SMILES: C[C@@H](C(=O)N(CCO[Si](C)(C)C(C)(C)C)c1cnc2c(c1)nc(-c1nn(COCC[Si](C)(C)C)c3c1C[C@@H]1C[C@]1(C)C3)n2COCC[Si](C)(C)C)N1CCOCC1]

A solution of Preparation 47a (120 mg, 0.18 mmol) in DMF (4 mL) at about 0° C. was treated with sodium hydride (60% suspension, 28 mg, 0.71 mmol). The mixture was stirred for about 30 min. The mixture was treated with (2-bromoethoxy)(tert-butyl)dimethylsilane (210 mg, 0.88 mmol). The reaction mixture was stirred at RT for about 20 h. Water (10 mL) was added and the resulting mixture was extracted with EtOAc (2×15 mL). The combined EtOAc extracts were dried, filtered and concentrated. The resulting residue was purified by chromatography (silica gel, EtOAc/PE (0.2% NH₄OH)=5-50%) to provide the title compound (60 mg, 41%). LC/MS m/z (M+H)⁺=840.4.

### Preparation 48

### N,2-Dimethyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide

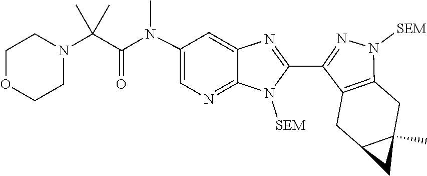 [SMILES: CN(C(=O)C(C)(C)N1CCOCC1)c1cnc2c(c1)nc(-c1nn(COCC[Si](C)(C)C)c3c1C[C@@H]1C[C@]1(C)C3)n2COCC[Si](C)(C)C]

### Step 1: 2-Methyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide (48a)

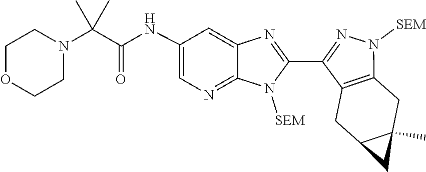 [SMILES: CC(C)(C(=O)Nc1cnc2c(c1)nc(-c1nn(COCC[Si](C)(C)C)c3c1C[C@@H]1C[C@]1(C)C3)n2COCC[Si](C)(C)C)N1CCOCC1]

A solution of Preparation 21 (96 mg, 0.56 mmol) in DMF (5 mL) was treated with iPr₂NEt (0.19 mL, 1.11 mmol) and HATU (211 mg, 0.56 mmol). The mixture was stirred at RT for about 1 h. The mixture was treated with Preparation 31 (200 mg, 0.37 mmol) and the mixture was stirred at about 50° C. for about 16 h. The mixture was treated with water (15 mL) and extracted with EtOAc (2×15 mL). The combined EtOAc extracts were dried, filtered and concentrated. The residue was combined with that from an identical reaction using 50 mg of Preparation 31 and similar proportions of other reagents. The combined residues were purified by chromatography (silica, EtOAc/PE 5-70%) to provide the title compound (130 mg, 40%). LC/MS m/z (M+H)⁺=696.3.

### Step 2: N,2-Dimethyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-morpholinopropanamide

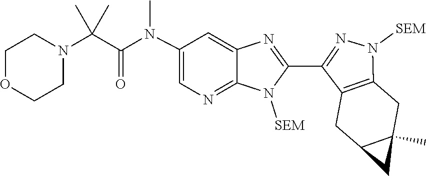 [SMILES: CN(C(=O)C(C)(C)N1CCOCC1)c1cnc2c(c1)nc(-c1nn(COCC[Si](C)(C)C)c3c1C[C@@H]1C[C@]1(C)C3)n2COCC[Si](C)(C)C]

A solution of Preparation 48a (130 mg, 0.19 mmol) in DMF (4 mL) at about 0° C. was treated with sodium hydride (60% suspension, 15 mg, 0.37 mmol). The mixture was stirred at about 0° C. for about 20 min. The mixture was treated with methyl iodide (35 μL, 0.56 mmol) at about 0° C. and the reaction mixture was stirred at RT for about 2 h. The reaction mixture was quenched with water (10 mL). The mixture was extracted with EtOAc (20 mL). The EtOAc layer was dried, filtered and concentrated to provide the title compound (140 mg, 90%). LC/MS m/z (M+H)⁺=710.4.

### Preparation 49

### 2-(6,6-Dimethyl-4,5,6,7-tetrahydro-1H-indazol-3-yl)-3H-imidazo[4,5-b]pyridin-6-amine

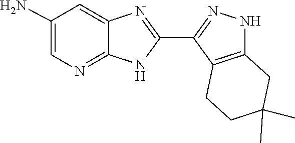 [SMILES: CC1(C)CCc2c(-c3nc4cc(N)cnc4[nH]3)n[nH]c2C1]

### Step 1: tert-Butyl (5,6-diaminopyridin-3-yl)carbamate (49a)

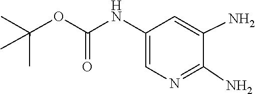 [SMILES: CC(C)(C)OC(=O)Nc1cnc(N)c(N)c1]

A solution of Preparation 25c (1.80 g, 4.14 mmol) in ethanol (150 mL) was treated with 5% Pd/C (Johnson Matthey cat #: 5R39, 1.80 g) at RT. The reaction mixture was stirred at about 70° C. and stirred under an atmosphere of hydrogen at 125 PSI for about 48 h. The reaction mixture was cooled to RT and filtered through Celite©. The filter cake was washed with ethanol (3×100 mL). The filtrate was concentrate, and the residue was purified by chromatography to provide the title compound (560 mg, 60%). 1H NMR (400 MHz, CD₃OD) δ 7.57 (s, 1H), 7.09 (d, J=2.1 Hz, 1H), 1.50 (s, 9H); LC/MS m/z (M+H)⁺=225.1.

### Step 2: tert-Butyl (2-(6,6-dimethyl-1-((2-(trimethylsilyl)ethoxy)methyl)-4,5,6,7-tetrahydro-1H-indazol-3-yl)-3H-imidazo[4,5-b]pyridin-6-yl)carbamate (49b)

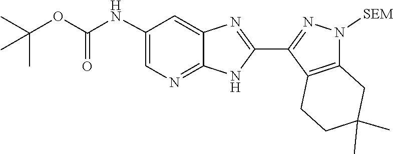 [SMILES: CC1(C)CCc2c(-c3nc4cc(NC(=O)OC(C)(C)C)cnc4[nH]3)nn(COCC[Si](C)(C)C)c2C1]

A solution of Preparation 49a (670 mg, 3.0 mmol) and sodium metabisulfite (290 mg, 1.5 mmol) in DMF (12 mL) was treated with Preparation 19 (970 mg, 3.15 mmol) and DMSO (0.53 mL, 7.5 mmol). The reaction mixture was stirred at about 110° C. for about 16 h. The reaction mixture was cooled to RT, diluted with EtOAc (150 mL) and washed with sat. aq. NaHCO₃ (50 mL) and brine (50 mL). The EtOAc extract was dried (MgSO₄), filtered and concentrated. The crude product was purified by chromatography to provide the title compound (600 mg, 39%). 1H NMR (400 MHz, CD₃OD) δ 8.27 (d, 2H), 5.46 (s, 2H), 3.68-3.54 (m, 2H), 2.94 (t, 2H), 2.54 (s, 2H), 1.63 (t, 2H), 1.55 (s, 9H), 1.08 (s, 6H), 0.96-0.83 (m, 2H), −0.04 (s, 9H); LC/MS m/z (M+H)⁺=513.3.

### Step 3: 2-(6,6-Dimethyl-4,5,6,7-tetrahydro-1H-indazol-3-yl)-3H-imidazo[4,5-b]pyridin-6-amine

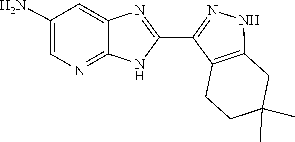 [SMILES: CC1(C)CCc2c(-c3nc4cc(N)cnc4[nH]3)n[nH]c2C1]

A solution of Preparation 49b (100 mg, 0.19 mmol) in 4M HCl in dioxane (1.5 mL) was stirred at about 60° C. for about 24 h. The reaction mixture was cooled to RT and concentrated. The residue was stirred with methanol (10 mL) and potassium carbonate (670 mg, 4.88 mmol) at RT for about 2 h. The solvent was removed by evaporation and residue was dissolved in 20% MeOH/DCM v/v (100 mL) and water (100 mL). The organic layer was separated and washed with brine, dried (MgSO₄), filtered and concentrated. The residue was purified by prep-HPLC (Column: Waters Sunfire C18, 19×100 mm, 5 μm; Mobile Phase A: 0.05% TFA in water (v/v); Mobile Phase B: 0.05% TFA in MeCN (v/v), Gradient: 5% B to 100% B over 8.5 min, HOLD at 100% B for 1.5 min, Flow: 25 mL/min) to provide the title compound (10 mg, 19%). 1H NMR (400 MHz, CD₃OD) δ 7.95 (d, 1H), 7.57 (d, 1H), 2.91 (t, 2H), 2.51 (s, 2H), 1.65 (t, 2H), 1.07 (s, 6H); LC/MS m/z (M+H)⁺=283.3.

### EXAMPLES

### Example 1

### (R)-N-Methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide

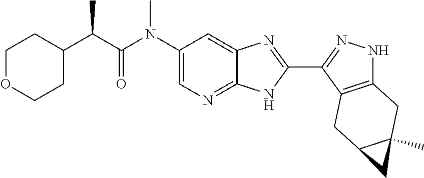 [SMILES: C[C@@H](C(=O)N(C)c1cnc2[nH]c(-c3n[nH]c4c3C[C@@H]3C[C@]3(C)C4)nc2c1)C1CCOCC1]

### Step 1: (R)-N-Methyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide

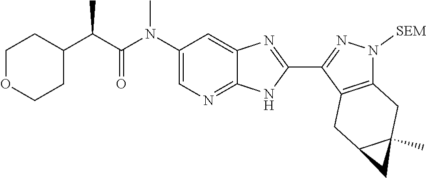 [SMILES: C[C@@H](C(=O)N(C)c1cnc2[nH]c(-c3nn(COCC[Si](C)(C)C)c4c3C[C@@H]3C[C@]3(C)C4)nc2c1)C1CCOCC1]

A solution of Preparation 28 (2.26 g, 5.33 mmol) and Preparation 21 (1.39 g, 5.33 mmol) in pyridine (76 mL) was treated with EDCl (2.04 g, 10.7 mmol) at RT. The reaction mixture was stirred for about 16 h at RT. Water was added (100 mL) and the mixture was extracted into EtOAc (2×80 mL). The EtOAc extracts were combined and concentrated. The resulting residue was purified by chromatography (silica, EtOAc/PE=0-100%) to provide the title compound (2.4 g, 80%). ¹H NMR (400 MHz, CD₃OD) δ=8.41-8.13 (m, 1H), 8.01-7.79 (m, 1H), 5.67-5.32 (m, 3H), 3.97-3.78 (m, 2H), 3.69-3.58 (m, 2H), 3.58-3.42 (m, 2H), 3.34 (s, 3H), 3.25-3.10 (m, 2H), 2.83-2.73 (m, 1H), 2.21-2.10 (m, 1H), 1.85-1.69 (m, 1H), 1.67-1.56 (m, 2H), 1.31 (s, 3H), 1.27-1.11 (m, 3H), 1.09-1.05 (m, 2H), 1.05-0.93 (m, 1H), 0.93-0.83 (m, 2H), 0.51-0.41 (m, 1H), 0.31-0.23 (m, 1H), −0.04 (s, 9H); LC/MS m/z (M+H)⁺=565.3.

### Step 2: (R)-N-Methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide

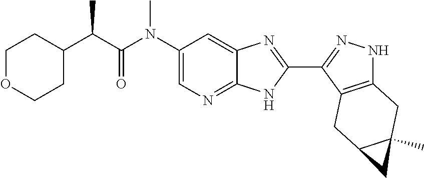 [SMILES: C[C@@H](C(=O)N(C)c1cnc2[nH]c(-c3n[nH]c4c3C[C@@H]3C[C@]3(C)C4)nc2c1)C1CCOCC1]

A solution of Example 1 Step 1 (2.4 g, 4.25 mmol) in TFA (42.5 mL) was treated with triethylsilane (2.47 g, 21.2 mmol) at about 5° C. The reaction mixture was stirred at RT for about 2 h. The mixture was concentrated and made basic to about pH 8 with sat. aq. NaHCO₃. The resulting mixture was extracted with EtOAc (2×30 mL). The combined EtOAc extracts were concentrated. The resulting residue was purified by prep-HPLC (Column YMC Triart C18 250×50 mm, 7 μm; Mobile phase A: water (0.05% v/v conc. NH₄OH); Mobile phase B: MeCN; 22-62% B gradient, 12 min; flow rate 60 mL/min) to provide the title compound (1.35 g, 73%). ¹H NMR (400 MHz, CD₃OD) δ=8.29 (br s, 1H), 8.05-7.74 (m, 1H), 3.93-3.80 (m, 2H), 3.53-3.36 (m, 3H), 3.34 (s, 4H), 3.19-3.00 (m, 2H), 2.84-2.70 (m, 1H), 2.21-2.08 (m, 1H), 1.86-1.70 (m, 1H), 1.66-1.55 (m, 2H), 1.31 (s, 3H), 1.27-1.12 (m, 2H), 1.09-1.05 (m, 2H), 1.05-0.90 (m, 1H), 0.48-0.37 (m, 1H), 0.30-0.21 (m, 1H); LC/MS m/z (M+H)⁺=435.1.

### Example 2

### 2,2-Difluoro-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)acetamide

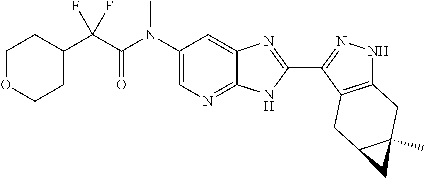 [SMILES: CN(C(=O)C(F)(F)C1CCOCC1)c1cnc2[nH]c(-c3n[nH]c4c3C[C@@H]3C[C@]3(C)C4)nc2c1]

### Step 1: 2,2-difluoro-N-methyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-yl)-2-(tetrahydro-2H-pyran-4-yl)acetamide

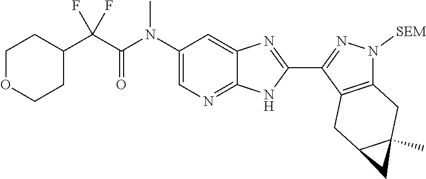 [SMILES: CN(C(=O)C(F)(F)C1CCOCC1)c1cnc2[nH]c(-c3nn(COCC[Si](C)(C)C)c4c3C[C@@H]3C[C@]3(C)C4)nc2c1]

A solution of Preparation 28 (1.00 g, 2.36 mmol) in pyridine (20 mL) was treated with EDCl (1.81 g, 9.42 mmol) and 2,2-difluoro-2-(tetrahydro-2H-pyran-4-yl)acetic acid (0.85 g, 4.71 mmol) at RT. The reaction mixture was stirred for about 2 h. The mixture was combined with that from an identical reaction using 100 mg of Preparation 28 and similar proportions of other reagents. The combined reaction mixtures were washed with H₂O (30 mL) and extracted with EtOAc (2×30 mL). The combined EtOAc extracts were dried (Na₂SO₄), filtered, and concentrated. The resulting residue was purified by chromatography to provide the title compound (1.10 g, 72%). LC/MS m/z (M+H)⁺=587.3.

### Step 2: 2,2-Difluoro-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)acetamide

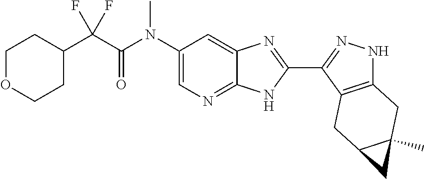 [SMILES: CN(C(=O)C(F)(F)C1CCOCC1)c1cnc2[nH]c(-c3n[nH]c4c3C[C@@H]3C[C@]3(C)C4)nc2c1]

A solution of Example 2 Step 1 (1.00 g, 1.70 mmol) in TFA (10 mL) was treated with triethylsilane (1.36 mL, 8.52 mmol) at RT and stirred for about 2 h. The reaction mixture was concentrated and the residue was treated with sat. aq. NaHCO₃ until the pH was about 8-9. Water (30 mL) was added and the mixture was extracted with EtOAc (30 mL). The EtOAc extract was dried (Na₂SO₄) and concentrated. The resulting residue was purified by chromatography to provide the title compound (460 mg, 59%). ¹H NMR (400 MHz, CD₃OD) δ=8.43-8.18 (m, 1H), 7.93 (d, 1H), 4.09-3.86 (m, 2H), 3.52-3.42 (m, 1H), 3.40 (s, 3H), 3.37-3.32 (m, 2H), 3.20-3.02 (m, 2H), 2.78 (d, 1H), 2.52-2.31 (m, 1H), 1.70-1.58 (m, 2H), 1.54-1.37 (m, 2H), 1.30 (s, 3H), 1.22-1.12 (m, 1H), 0.48-0.38 (m, 1H), 0.27 (t, 1H); LC/MS m/z (M+H)⁺=457.2.

The following examples were prepared in a similar fashion to Examples 1 and 2, where observed LC-MS m/z is shown in the table below as [M+H]⁺.

| Ex | Structure and Name | [M + H]+ |
|---|---|---|
| 3 | 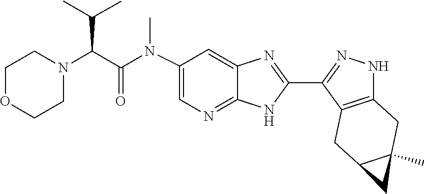 [SMILES: CC(C)[C@@H](C(=O)N(C)c1cnc2[nH]c(-c3n[nH]c4c3C[C@@H]3C[C@]3(C)C4)nc2c1)N1CCOCC1] | 464.3 |
| | (S)-N,3-Dimethyl-N-(2-((4aR,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinobutanamide | |
| 4 | 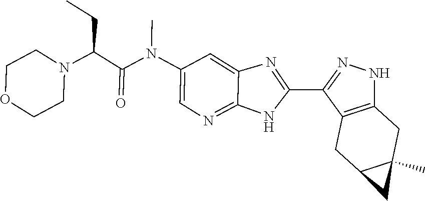 [SMILES: CC[C@@H](C(=O)N(C)c1cnc2[nH]c(-c3n[nH]c4c3C[C@@H]3C[C@]3(C)C4)nc2c1)N1CCOCC1] | 450.3 |
| | (S)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinobutanamide | |
| 5 | 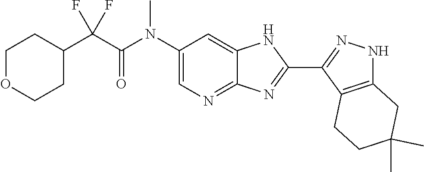 [SMILES: CN(C(=O)C(F)(F)C1CCOCC1)c1cnc2nc(-c3n[nH]c4c3CCC(C)(C)C4)[nH]c2c1] | 459.1 |
| | N-(2-(6,6-Dimethyl-4,5,6,7-tetrahydro-1H-indazol-3-yl)-1H- | |
| | imidazo[4,5-b]pyridin-6-yl)-2,2-difluoro-N-methyl-2-(tetrahydro-2H- | |
| | pyran-4-yl)acetamide | |
| 6 | 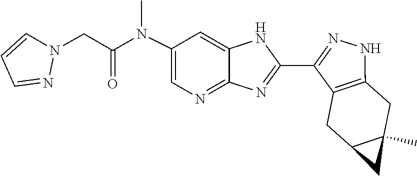 [SMILES: CN(C(=O)Cn1cccn1)c1cnc2nc(-c3n[nH]c4c3C[C@@H]3C[C@]3(C)C4)[nH]c2c1] | 403.2 |
| | N-Methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (1H-pyrazol-1-yl)acetamide | |
| 7 | 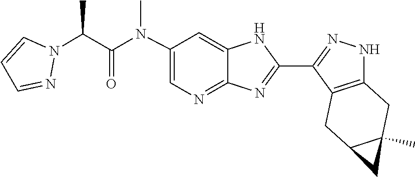 [SMILES: C[C@@H](C(=O)N(C)c1cnc2nc(-c3n[nH]c4c3C[C@@H]3C[C@]3(C)C4)[nH]c2c1)n1cccn1] | 417.1 |
| | (S)-N-Methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (1H-pyrazol-1-yl)propenamide | |
| 8 | 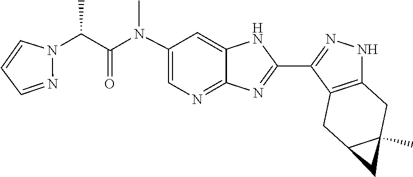 [SMILES: C[C@H](C(=O)N(C)c1cnc2nc(-c3n[nH]c4c3C[C@@H]3C[C@]3(C)C4)[nH]c2c1)n1cccn1] | 417.1 |
| | (R)-N-Methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (1H-pyrazol-1-yl)propanamide | |
| 9 | 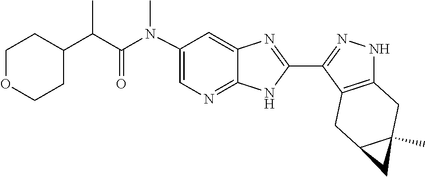 [SMILES: CC(C(=O)N(C)c1cnc2[nH]c(-c3n[nH]c4c3C[C@@H]3C[C@]3(C)C4)nc2c1)C1CCOCC1] | 435.1 |
| | N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | (tetrahydro-2H-pyran-4-yl)propanamide | |
| 10 | 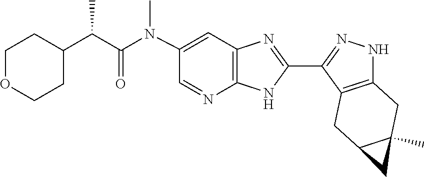 [SMILES: C[C@H](C(=O)N(C)c1cnc2[nH]c(-c3n[nH]c4c3C[C@@H]3C[C@]3(C)C4)nc2c1)C1CCOCC1] | 435.1 |
| | (S)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | (tetrahydro-2H-pyran-4-yl)propanamide | |
| 11 | 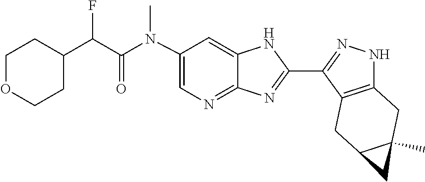 [SMILES: CN(C(=O)C(F)C1CCOCC1)c1cnc2nc(-c3n[nH]c4c3C[C@@H]3C[C@]3(C)C4)[nH]c2c1] | 439.2 |
| | 2-Fluoro-N-methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (tetrahydro-2H-pyran-4-yl)acetamide | |
| 12 | 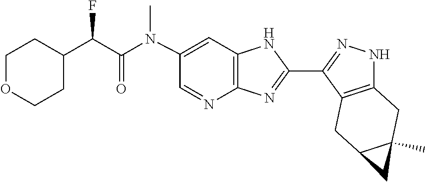 [SMILES: CN(C(=O)[C@H](F)C1CCOCC1)c1cnc2nc(-c3n[nH]c4c3C[C@@H]3C[C@]3(C)C4)[nH]c2c1] | 439.2 |
| | (R)-2-Fluoro-N-methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (tetrahydro-2H-pyran-4-yl)acetamide | |
| 13 | 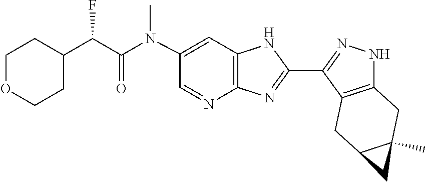 [SMILES: CN(C(=O)[C@@H](F)C1CCOCC1)c1cnc2nc(-c3n[nH]c4c3C[C@@H]3C[C@]3(C)C4)[nH]c2c1] | 439.2 |
| | (S)-2-Fluoro-N-methyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | (tetrahydro-2H-pyran-4-yl)acetamide | |

### Example 14

### (S)-N-(2-(6,6-Dimethyl-4,5,6,7-tetrahydro-1H-indazol-3-yl)-3H-imidazo[4,5-b]pyridin-6-yl)-N-methyl-2-morpholinopropanamide

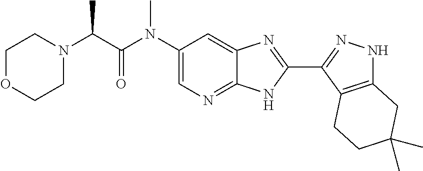 [SMILES: C[C@@H](C(=O)N(C)c1cnc2[nH]c(-c3n[nH]c4c3CCC(C)(C)C4)nc2c1)N1CCOCC1]

A solution of Preparation 49 (100 mg, 0.36 mmol) and Preparation 20 (110 mg, 0.71 mmol) in pyridine (4 mL) was treated with EDCl (270 mg, 1.42 mmol) at RT. The mixture was stirred at RT for about 16 h. The reaction mixture was diluted with water and extracted with EtOAc (2×50 mL). The combined EtOAc extracts were washed with brine, dried (MgSO₄), filtered and concentrated. The residue was stirred with 1:1 v/v methanol/THF (20 mL) and potassium carbonate (490 mg, 3.55 mmol) for about 2 h. The mixture was concentrated and residue was taken up in water (100 mL) and extracted with EtOAc (100 mL). The EtOAc extract was washed with brine, dried (MgSO₄), filtered and concentrated. The crude product was purified by prep HPLC (Column: Waters Sunfire C18, 19×100 mm, 5 μm; Mobile Phase A: 0.05% TFA in water (v/v); Mobile Phase B: 0.05% TFA in MeCN (v/v), Gradient: 5% B to 70% B over 8.5 min, Hold at 100% B for 1.5 min, Flow: 25 mL/min) to provide the title compound (60 mg, 44%). 1H NMR (400 MHz, CD₃OD) δ 8.56-8.48 (m, 2H), 4.08 (q, 1H), 3.98 (s, 4H), 3.43 (d, 4H), 2.94 (t, 2H), 2.50 (s, 2H), 1.72 (d, 3H), 1.65 (t, 2H), 1.08 (s, 6H); LC/MS m/z (M+H)⁺=424.2.

### Example 15

### (S)-N-(2-Hydroxyethyl)-N-(2-((4aS,5aR)-5a-methyl-1,4,4a,5,5a,6-hexahydrocyclopropa[f]indazol-3-yl)-3H-imidazo[4,5-b]pyridin-6-yl)-2-morpholinopropanamide

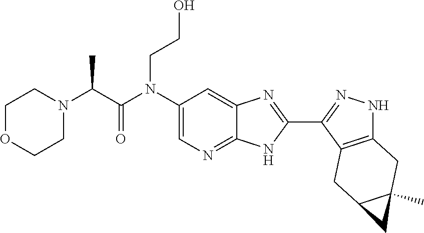 [SMILES: C[C@@H](C(=O)N(CCO)c1cnc2[nH]c(-c3n[nH]c4c3C[C@@H]3C[C@]3(C)C4)nc2c1)N1CCOCC1]

A solution of Preparation 47 (60 mg, 0.07 mmol) in DCM (3 mL) was treated with TFA (1 mL) at RT. The reaction mixture was stirred for 2 h. Additional TFA (2 mL) was added to the mixture and it was stirred at RT for about 1 h. The reaction mixture was concentrated. The resulting residue was dissolved in methanol (5 mL), treated with NH₄OH at about 30° C. and stirred for about 16 h. The mixture was concentrated and combined with that from an identical reaction using 15 mg Preparation 47 and similar proportions of other reagents. The combined residues were purified by prep-HPLC (Column: Phenomenex Gemini-NX C18 75×30 mm, 3 μm; Gradient: Mobile phase A: Water (0.225% Formic Acid); Mobile phase B: MeCN; 0-100% B gradient, Flow Rate=25 mL/min) to provide the title compound (18 mg, 38%). ¹H NMR (400 MHz, CD₃OD) δ=8.56-8.25 (m, 1H), 8.06 (br s, 1H), 4.00-3.83 (m, 2H), 3.77-3.70 (m, 2H), 3.68-3.57 (m, 4H), 3.43 (d, 1H), 3.21 (q, 1H), 3.18-3.04 (m, 2H), 2.78 (d, 1H), 2.63-2.53 (m, 2H), 2.43-2.33 (m, 2H), 1.30 (s, 3H), 1.18 (d, 4H), 0.47-0.39 (m, 1H), 0.25 (t, 1H). LC/MS m/z (M+H)⁺=466.2.

The following examples were prepared in a similar fashion to Example 15, where observed LC-MS m/z is shown in the table below as [M+H]⁺.

| Ex | Structure and Name | [M + H]+ |
|---|---|---|
| 16 | 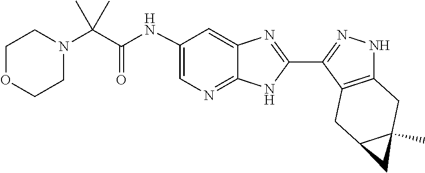 [SMILES: CC(C)(C(=O)Nc1cnc2[nH]c(-c3n[nH]c4c3C[C@@H]3C[C@]3(C)C4)nc2c1)N1CCOCC1] | 450.1 |
| | 2-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinopropanamide | |
| 17 | 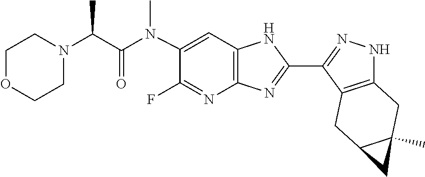 [SMILES: C[C@@H](C(=O)N(C)c1cc2[nH]c(-c3n[nH]c4c3C[C@@H]3C[C@]3(C)C4)nc2nc1F)N1CCOCC1] | 454.2 |
| | (S)-N-(5-Fluoro-2-((4aS,5aR)-5a-methyl-1,4,4a,5,5a,6- | |
| | hexahydrocyclopropa[f]indazol-3-yl)-1H-imidazo[4,5-b]pyridin-6-yl)-N- | |
| | methyl-2-morpholinopropanamide | |

### Example 18

### (R)-N-(2-(6,6-Dimethyl-4,5,6,7-tetrahydro-1H-indazol-3-yl)-3H-imidazo[4,5-b]pyridin-6-yl)-N-ethyl-2-(tetrahydro-2H-pyran-4-yl)ProPenamide

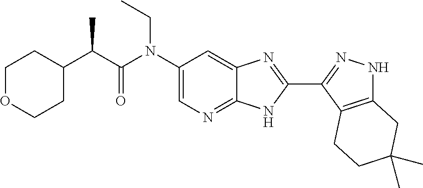 [SMILES: CCN(C(=O)[C@H](C)C1CCOCC1)c1cnc2[nH]c(-c3n[nH]c4c3CCC(C)(C)C4)nc2c1]

### Step 1: (R)-N-(2-(6,6-Dimethyl-1-((2-(trimethylsilyl)ethoxy)methyl)-4,5,6,7-tetrahydro-1H-indazol-3-yl)-3H-imidazo[4,5-b]pyridin-6-yl)-N-ethyl-2-(tetrahydro-2H-pyran-4-yl)propenamide

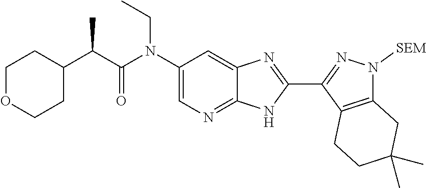 [SMILES: CCN(C(=O)[C@H](C)C1CCOCC1)c1cnc2[nH]c(-c3nn(COCC[Si](C)(C)C)c4c3CCC(C)(C)C4)nc2c1]

A solution of Preparation 19 (371 mg, 1.20 mmol) in DMF (10 mL) and DMSO (2 mL) was treated with Preparation 39 (320 mg, 1.09 mmol) and sodium metabisulfite (104 mg, 0.55 mmol) at about 30° C. The reaction mixture was stirred at about 110° C. for about 18 h. Water (40 mL) was added and the mixture was filtered. The filter cake was washed with water (2×15 mL). The solids were collected to provide the title compound (500 mg, 79%). LC/MS m/z (M+H)⁺=581.3.

### Step 2: (R)-N-(2-(6,6-Dimethyl-4,5,6,7-tetrahydro-1H-indazol-3-yl)-3H-imidazo[4,5-b]pyridin-6-yl)-N-ethyl-2-(tetrahydro-2H-pyran-4-yl)propenamide

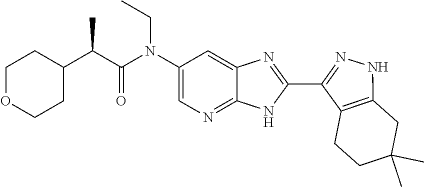 [SMILES: CCN(C(=O)[C@H](C)C1CCOCC1)c1cnc2[nH]c(-c3n[nH]c4c3CCC(C)(C)C4)nc2c1]

A solution of Example 19 Step 1 (500 mg, 0.86 mmol) in TFA (5 mL) was treated with triethylsilane (0.28 mL, 1.72 mmol) and the mixture was stirred at about 30° C. for about 1 h. The mixture was concentrated and the residue was treated with sat. aq. NaHCO₃ to about pH 8 and extracted with EtOAc (2×15 mL). The combined organic extracts were dried (Na₂SO₄), filtered and concentrated. The crude product was purified by prep-HPLC (Column: Boston Prime C18 150×30 mm, 5 μm; Mobile phase A: water (0.2% formic acid); Mobile phase B: MeCN; Gradient: 40-60% B; Flow rate=25 ml/min) to provide the title compound (125 mg, 32%). ¹H NMR (400 MHz, CDCl₃) δ=14.96-14.58 (m, 1H), 8.35-8.11 (m, 1H), 8.03-7.87 (m, 1H), 4.04-3.75 (m, 4H), 3.48-3.27 (m, 2H), 3.19-3.06 (m, 2H), 2.79-2.63 (m, 2H), 2.16-2.03 (m, 1H), 1.94-1.80 (m, 1H), 1.74 (t, 3H), 1.64-1.56 (m, 2H), 1.36-1.24 (m, 1H), 1.20 (t, 3H), 1.15 (s, 6H), 1.11-1.04 (m, 3H), 1.03-0.97 (m, 1H).

The following examples were prepared in a similar fashion to Example 18, where observed LC-MS m/z is shown in the table below as [M+H]⁺.

| Ex | Structure and Name | [M + H]+ |
|---|---|---|
| 19 | 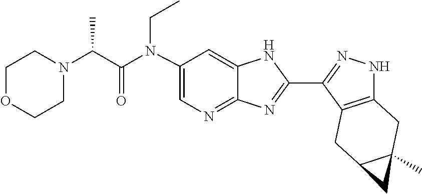 [SMILES: CCN(C(=O)[C@@H](C)N1CCOCC1)c1cnc2nc(-c3n[nH]c4c3C[C@@H]3C[C@]3(C)C4)[nH]c2c1] | 449.3 |
| | (R)-N-Ethyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | (tetrahydro-2H-pyran-4-yl)propenamide | |
| 20 | 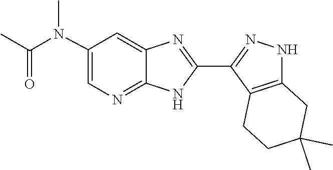 [SMILES: CC(=O)N(C)c1cnc2[nH]c(-c3n[nH]c4c3CCC(C)(C)C4)nc2c1] | 339.2 |
| | N-(2-(6,6-Dimethyl-4,5,6,7-tetrahydro-1H-indazol-3-yl)-3H- | |
| | imidazo[4,5-b]pyridin-6-yl)-N-methylacetamide | |
| 21 | 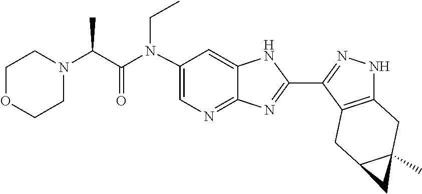 [SMILES: CCN(C(=O)[C@H](C)N1CCOCC1)c1cnc2nc(-c3n[nH]c4c3C[C@@H]3C[C@]3(C)C4)[nH]c2c1] | 450.1 |
| | (S)-N-Ethyl-N-(2-((4aS,5aR)-5a-methyl-1,4,4a,5,5a,6- | |
| | hexahydrocyclopropa[f]indazol-3-yl)-1H-imidazo[4,5-b]pyridin-6-yl)-2- | |
| | morpholinopropanamide | |
| 22 | 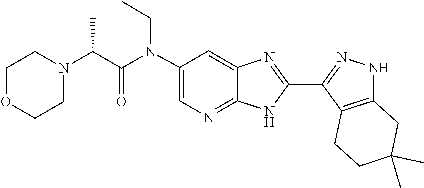 [SMILES: CCN(C(=O)[C@@H](C)N1CCOCC1)c1cnc2[nH]c(-c3n[nH]c4c3CCC(C)(C)C4)nc2c1] | 452.3 |
| | (R)-N-(2-(6,6-Dimethyl-4,5,6,7-tetrahydro-1H-indazol-3-yl)-3H- | |
| | imidazo[4,5-b]pyridin-6-yl)-N-ethyl-2-morpholinopropanamide | |
| 23 | 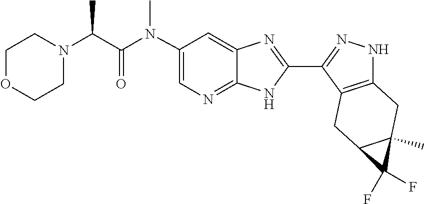 [SMILES: C[C@@H](C(=O)N(C)c1cnc2[nH]c(-c3n[nH]c4c3C[C@@H]3C(F)(F)[C@]3(C)C4)nc2c1)N1CCOCC1] | 472.4 |
| | (S)-N-(2-((4aS,5aR)-5,5-Difluoro-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinopropanamide | |
| 24 | 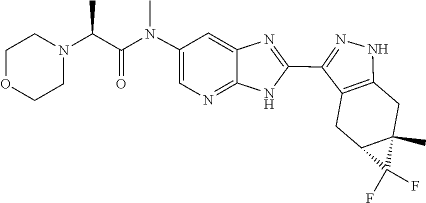 [SMILES: C[C@@H](C(=O)N(C)c1cnc2[nH]c(-c3n[nH]c4c3C[C@H]3C(F)(F)[C@@]3(C)C4)nc2c1)N1CCOCC1] | 472.4 |
| | (S)-N-(2-((4aR,5aS)-5,5-Difluoro-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinopropanamide | |
| 25 | 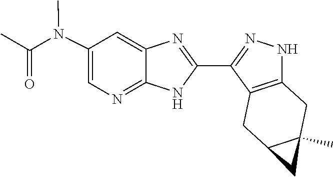 [SMILES: CC(=O)N(C)c1cnc2[nH]c(-c3n[nH]c4c3C[C@@H]3C[C@]3(C)C4)nc2c1] | 337.2 |
| | N-methyl-N-(2-((4aS,5aR)-5a-Methyl-1,4,4a,5,5a,6- | |
| | hexahydrocyclopropa[f]indazol-3-yl)-1H-imidazo[4,5-b]pyridin-6- | |
| | yl)acetamide | |
| 26 | 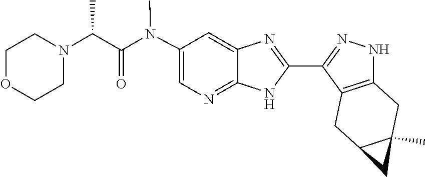 [SMILES: C[C@H](C(=O)N(C)c1cnc2[nH]c(-c3n[nH]c4c3C[C@@H]3C[C@]3(C)C4)nc2c1)N1CCOCC1] | 463.4 |
| | (R)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinopropanamide | |
| 27 | 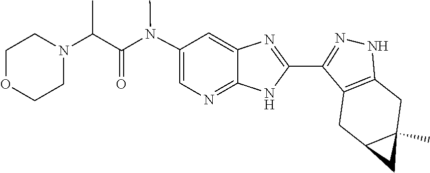 [SMILES: CC(C(=O)N(C)c1cnc2[nH]c(-c3n[nH]c4c3C[C@@H]3C[C@]3(C)C4)nc2c1)N1CCOCC1] | 436.2 |
| | N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinopropanamide | |
| 28 | 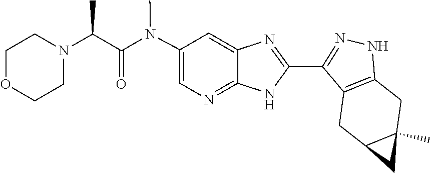 [SMILES: C[C@@H](C(=O)N(C)c1cnc2[nH]c(-c3n[nH]c4c3C[C@@H]3C[C@]3(C)C4)nc2c1)N1CCOCC1] | 436.4 |
| | (S)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | morpholinopropanamide | |
| 29 | 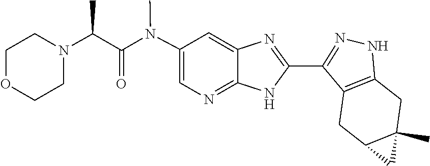 [SMILES: C[C@@H](C(=O)N(C)c1cnc2[nH]c(-c3n[nH]c4c3C[C@H]3C[C@@]3(C)C4)nc2c1)N1CCOCC1] | 436.4 |
| | (2S)-N-Methyl-N-(2-(5a-methyl-1,4,4a,5,5a,6- | |
| | hexahydrocyclopropa[f]indazol-3-yl)-3H-imidazo[4,5-b]pyridin-6-yl)-2- | |
| | morpholinopropanamide | |
| 30 | 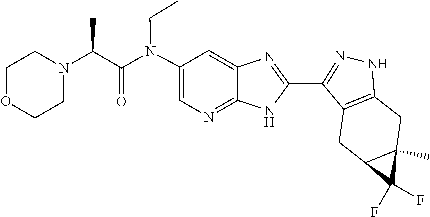 [SMILES: CCN(C(=O)[C@H](C)N1CCOCC1)c1cnc2[nH]c(-c3n[nH]c4c3C[C@@H]3C(F)(F)[C@]3(C)C4)nc2c1] | 486.3 |
| | (S)-N-(2-((4aS,5aR)-5,5-Difluoro-5a-methyl-1,4,4a,5,5a,6- | |
| | hexahydrocyclopropa[f]indazol-3-yl)-3H-imidazo[4,5-b]pyridin-6-yl)-N- | |
| | ethyl-2-morpholinopropanamide | |

### Example 31

### (R)-N-(2-Hydroxyethyl)-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide

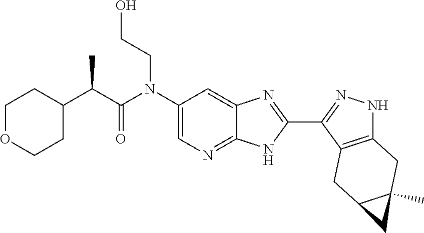 [SMILES: C[C@@H](C(=O)N(CCO)c1cnc2[nH]c(-c3n[nH]c4c3C[C@@H]3C[C@]3(C)C4)nc2c1)C1CCOCC1]

### Step 1: (R)-N-(2-Hydroxyethyl)-N-(2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-2-(tetrahydro-2H-pyran-4-yl)propenamide

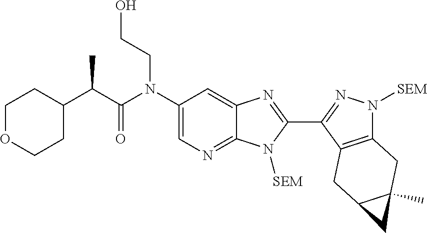 [SMILES: C[C@@H](C(=O)N(CCO)c1cnc2c(c1)nc(-c1nn(COCC[Si](C)(C)C)c3c1C[C@@H]1C[C@]1(C)C3)n2COCC[Si](C)(C)C)C1CCOCC1]

A solution of Preparation 40 (200 mg, 0.34 mmol) in THF (7 mL) was treated with iPr₂NEt (0.18 mL, 1.03 mmol), Preparation 21 (162 mg, 1.03 mmol) and CMPI (262 mg, 1.03 mmol). The reaction mixture was stirred at about 60° C. for about 16 h. Water (15 mL) was added to the reaction mixture. The mixture was extracted into EtOAc (15 mL×2). The organic extracts were combined, dried, and concentrated. The resulting residue was purified by chromatography to provide the title compound (120 mg, 48%). LC/MS m/z (M+H)⁺=725.3.

### Step 2: (R)-N-(2-Hydroxyethyl)-N-(2-((4aS,5aR)-5a-methyl-1,4,4a,5,5a,6-hexahydrocyclopropa[f]indazol-3-yl)-3H-imidazo[4,5-b]pyridin-6-yl)-2-(tetrahydro-2H-pyran-4-yl)propenamide

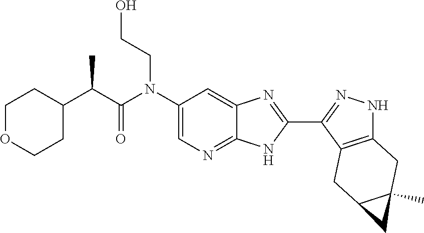 [SMILES: C[C@@H](C(=O)N(CCO)c1cnc2[nH]c(-c3n[nH]c4c3C[C@@H]3C[C@]3(C)C4)nc2c1)C1CCOCC1]

A solution of Example 31 Step 1 (100 mg, 0.14 mmol) in DCM (5 mL) was treated with TFA (1 mL) at about 0° C. and the mixture was stirred at RT for about 2 h. The reaction mixture was diluted with DCM (10 mL) and concentrated. The residue was taken up in methanol (5 mL) and treated with conc. NH₄OH (1.5 mL) and the mixture was stirred at RT for about 2 h. The reaction mixture was concentrated and the residue was purified by prep-HPLC (Column: Boston Prime C18 150×30 mm, 5 μm; Mobile phase A: water (0.05% v/v conc. NH₄OH); Mobile phase B: MeCN; 30-53% B gradient, 10 min; flow rate 25 mL/min) to provide the title compound (10 mg, 15%). ¹H NMR (400 MHz, CD₃OD) δ=8.31 (br s, 1H), 7.97 (br s, 1H), 4.61 (s, 1H), 4.05-3.79 (m, 4H), 3.79-3.68 (m, 2H), 3.50-3.40 (m, 1H), 3.39-3.33 (m, 2H), 3.20-3.02 (m, 2H), 2.77 (d, 1H), 2.17-2.05 (m, 1H), 1.86-1.71 (m, 1H), 1.71-1.55 (m, 2H), 1.30 (s, 3H), 1.27-1.11 (m, 2H), 1.07 (d, 3H), 1.02-0.94 (m, 1H), 0.48-0.38 (m, 1H), 0.24 (t, 1H). LC/MS m/z (M+H)⁺=465.3.

### Example 32

### (R)-N-(2-(6,6-Dimethyl-4,5,6,7-tetrahydro-1H-indazol-3-yl)-3H-imidazo[4,5-b]pyridin-6-yl)-N-methyl-2-(tetrahydro-2H-pyran-4-yl)propenamide

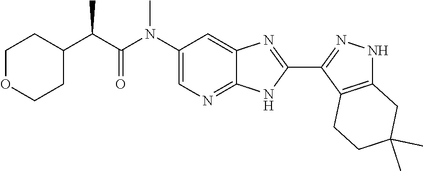 [SMILES: C[C@@H](C(=O)N(C)c1cnc2[nH]c(-c3n[nH]c4c3CCC(C)(C)C4)nc2c1)C1CCOCC1]

### Step 1: (R)-N-(2-(6,6-Dimethyl-1-((2-(trimethylsilyl)ethoxy)methyl)-4,5,6,7-tetrahydro-1H-indazol-3-yl)-3H-imidazo[4,5-b]pyridin-6-yl)-N-methyl-2-(tetrahydro-2H-pyran-4-yl)propanamide

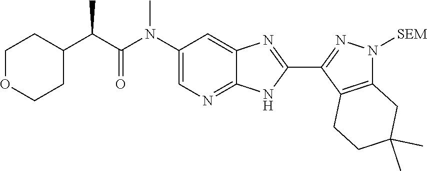 [SMILES: C[C@@H](C(=O)N(C)c1cnc2[nH]c(-c3nn(COCC[Si](C)(C)C)c4c3CCC(C)(C)C4)nc2c1)C1CCOCC1]

A solution of Preparation 35 (200 mg, 0.47 mmol) in THF (10 mL) was treated with Preparation 21 (0.0742 g, 0.469 mmol), CMPI (180 mg, 0.70 mmol) and iPr₂NEt (0.25 mL, 1.41 mmol) at about 30° C. The reaction mixture stirred at about 60° C. for about 18 h. Water was added and the mixture was extracted into EtOAc (3×). The combined EtOAc extracts were dried (Na₂SO₄), filtered, and concentrated. The resulting residue was purified by chromatography to provide the title compound (100 mg, 38%). LC/MS m/z (M+H)⁺=567.2.

### Step 2: (R)-N-(2-(6,6-Dimethyl-4,5,6,7-tetrahydro-1H-indazol-3-yl)-3H-imidazo[4,5-b]pyridin-6-yl)-N-methyl-2-(tetrahydro-2H-pyran-4-yl)propenamide

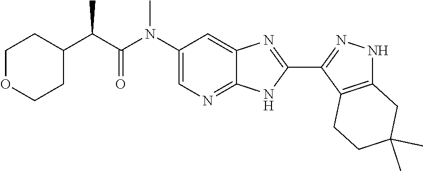 [SMILES: C[C@@H](C(=O)N(C)c1cnc2[nH]c(-c3n[nH]c4c3CCC(C)(C)C4)nc2c1)C1CCOCC1]

A solution of Example 32 Step 1 (100 mg, 0.18 mmol) in TFA (10 mL) was treated with triethylsilane (84 μL, 0.53 mmol) at about 30° C. and the mixture was stirred for about 2 h. The solvent was removed under reduced pressure. The residue was treated with sat. aq. NaHCO₃ and the mixture was extracted with EtOAc (2×). The combined EtOAc extracts were dried (Na₂SO₄), filtered, and concentrated. The resulting residue was purified by prep-HPLC (Column: Boston Prime C18 150×30 mm, 5 um; Mobile phase A: water (0.225% formic acid); Mobile phase B: MeCN; Gradient: 7-50% B); Flow Rate: 25 mL/min) to provide the title compound (34 mg, 44%). LC/MS m/z (M+H)⁺=437.3.

### Example 33

### (S)-N-(2-(6,6-Dimethyl-4,5,6,7-tetrahydro-1H-indazol-3-yl)-1H-benzo[d]imidazol-5-yl)-2-morpholinopropanamide

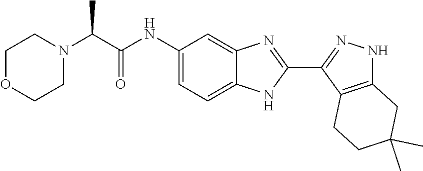 [SMILES: C[C@@H](C(=O)Nc1ccc2[nH]c(-c3n[nH]c4c3CCC(C)(C)C4)nc2c1)N1CCOCC1]

A solution of Preparation 49 (100 mg, 0.36 mmol) in pyridine (4 mL) was treated with Preparation 20 (113 mg, 0.71 mmol) and EDCl (272 mg, 1.42 mmol). The reaction mixture was stirred at RT for about 18h. The mixture was diluted with water and extracted with EtOAc (2×). The combined EtOAc extracts were washed with brine, dried (MgSO₄), filtered and concentrated. The residue was dissolved in a mixture of methanol and THF and treated with potassium carbonate (490 mg, 3.55 mmol) and the mixture was stirred at RT for about 4 h. The reaction mixture was concentrated and the residue was taken up in water and extracted with EtOAc. The EtOAc extract was washed with brine, dried (MgSO₄), filtered and concentrated. The crude product was purified by prep HPLC to provide the title compound (66 mg, 44%). HPLC method: Column: Waters Atlantis C18 4.6×100 mm, 5 μm; Mobile Phase A: 0.05% TFA in water (v/v); Mobile Phase B: 0.05% TFA in MeCN (v/v). Gradient: 5% B to 95% B over 4 min, hold at 95% B for 1 min. Flow rate=2 mL/min. RT=1.7 min; LC/MS m/z (M+H)⁺=424.5.

### Example 34

### (S)-N-Methyl-N-(5-methyl-2-((4aS,5aR)-5a-methyl-1,4,4a,5,5a,6-hexahydrocyclopropa[f]indazol-3-yl)-3H-imidazo[4,5-b]pyridin-6-yl)-2-morpholinopropanamide

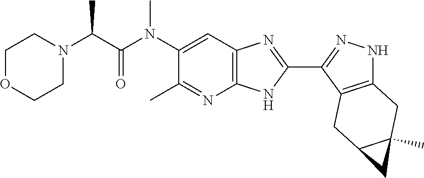 [SMILES: Cc1nc2[nH]c(-c3n[nH]c4c3C[C@@H]3C[C@]3(C)C4)nc2cc1N(C)C(=O)[C@H](C)N1CCOCC1]

A solution of Preparation 46 (23 mg, 75 μmol) and Preparation 20 (27 mg, 164 μmol) in pyridine (1 mL) was treated with EDCl (32 mg, 164 μmol). The reaction mixture was stirred at RT for about 80 h. The reaction mixture was treated with Preparation 20 (27 mg, 164 μmol) in pyridine (1 mL) and EDCl (32 mg, 164 μmol). The reaction mixture was stirred at about 60° C. for about 18 h. The reaction mixture was cooled to RT, treated with sat. aq. NaHCO₃ and washed with EtOAc (2×). The combined EtOAc extracts were dried (MgSO₄), filtered and concentrated. The residue was suspended in a mixture of DCM/heptane and concentrated. The residue was suspended in methanol and stirred with potassium carbonate at RT for about 45 min. The slurry was treated with water and the methanol evaporated under reduced pressure. The mixture was washed with EtOAc (2×). The combined EtOAc extracts were dried (MgSO₄), filtered and concentrated. The crude product was purified by prep HPLC (Waters Atlantis C18 4.6×100 mm, 5 μm; Mobile Phase A: 0.05% TFA in water (v/v); Mobile Phase B: 0.05% TFA in MeCN (v/v). Gradient: 95.0% water/5.0% MeCN linear to 5.0% water/95.0% MeCN in 4 min, HOLD at 5.0% water/95.0% MeCN to 5 min; Flow: 2 mL/min) to provide the title compound (2.4 mg, 7%). Retention time: 1.74 min; LC-MS m/z (M+H)⁺=450.6.

### Example 35

### N-(5-Methoxy-2-((4aS,5aR)-5a-methyl-1,4,4a,5,5a,6-hexahydrocyclopropa[f]indazol-3-yl)-3H-imidazo[4,5-b]pyridin-6-yl)-N-methyl-2-morpholinoacetamide

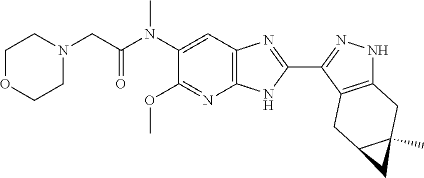 [SMILES: COc1nc2[nH]c(-c3n[nH]c4c3C[C@@H]3C[C@]3(C)C4)nc2cc1N(C)C(=O)CN1CCOCC1]

### Step 1: N-(5-Methoxy-2-((4aS,5aR)-5a-methyl-1-((2-(trimethylsilyl)ethoxy)methyl)-1,4,4a,5,5a,6-hexahydrocyclopropa[f]indazol-3-yl)-3-((2-(trimethylsilyl)ethoxy)methyl)-3H-imidazo[4,5-b]pyridin-6-yl)-N-methyl-2-morpholinoacetamide and N-(5-methoxy-2-((4aS,5aR)-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-imidazo[4,5-b]pyridin-6-yl)-N-methyl-2-morpholinoacetamide

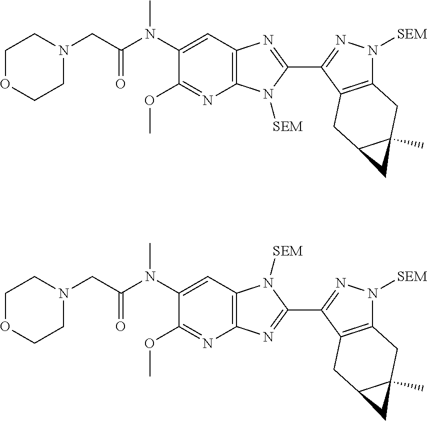 [SMILES: COc1nc2c(cc1N(C)C(=O)CN1CCOCC1)nc(-c1nn(COCC[Si](C)(C)C)c3c1C[C@@H]1C[C@]1(C)C3)n2COCC[Si](C)(C)C ; COc1nc2nc(-c3nn(COCC[Si](C)(C)C)c4c3C[C@@H]3C[C@]3(C)C4)n(COCC[Si](C)(C)C)c2cc1N(C)C(=O)CN1CCOCC1]

A solution of 2-morpholinoacetic acid (31 mg, 0.21 mmol), BTFFH (68 mg, 0.21 mmol) and iPr₂NEt (68 μL, 0.38 mmol) were dissolved in DMF (0.2 mL) and the solution was stirred at RT for about 25 min. Preparation 42 (50 mg, 0.085 mmol) was added as a solution in DMF (0.7 mL) and the mixture was stirred at about 100° C. for about 18 h. The reaction mixture was cooled to RT and diluted with water. The mixture was treated with sodium chloride and extracted with EtOAc (2×). The combined EtOAc extracts were dried (MgSO₄), filtered and concentrated. The residue was suspended in a mixture of DCM and heptane and concentrated. The residue was purified by chromatography (silica, EtOAc/heptane=20-100%) to provide the title compounds as a mixture of regioisomers (76 mg). LC-MS m/z (M+H)⁺=713.8.

### Step 2: N-(5-Methoxy-2-((4aS,5aR)-5a-methyl-1,4,4a,5,5a,6-hexahydrocyclopropa[f]indazol-3-yl)-3H-imidazo[4,5-b]pyridin-6-yl)-N-methyl-2-morpholinoacetamide

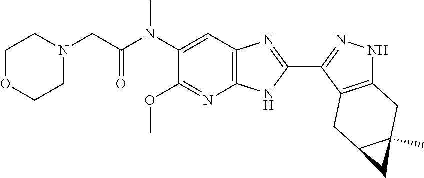 [SMILES: COc1nc2[nH]c(-c3n[nH]c4c3C[C@@H]3C[C@]3(C)C4)nc2cc1N(C)C(=O)CN1CCOCC1]

A solution of the compounds of Example 35 Step 1 (61 mg, 0.09 mmol) in TFA (1 mL) was treated with triethylsilane (70 μL, 0.43 mmol) and the mixture was stirred at RT for about 45 min. The mixture was diluted with toluene and evaporated under a stream of nitrogen. The residue was taken up in 1:4 (v/v) conc. NH₄OH/ethanol and stirred at RT for about 1 h. The reaction mixture was concentrated, diluted with water and extracted with EtOAc (2×). The combined EtOAc extracts were dried (MgSO₄), filtered and concentrated. The residue was purified by prep HPLC prep HPLC (Waters Atlantis C18 4.6×100 mm, 5 μm; Mobile Phase A: 0.05% TFA in water (v/v); Mobile Phase B: 0.05% TFA in MeCN (v/v). Gradient: 95.0% water/5.0% MeCN linear to 5.0% water/95.0% MeCN in 4 min, HOLD at 5.0% water/95.0% MeCN to 5 min; Flow: 2 mL/min) to provide the title compound (11 mg, 28%). Retention time: 1.84 min; LC-MS m/z (M+H)⁺=452.6.

The following Examples were prepared in a similar fashion to Example 35, where observed LC-MS m/z is shown in the table below as [M+H]⁺.

| Ex | Structure and Name | [M + H]+ |
|---|---|---|
| 36 | 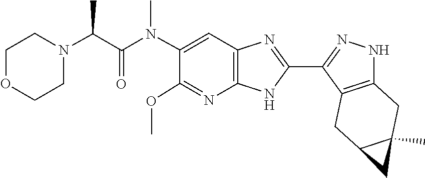 [SMILES: COc1nc2[nH]c(-c3n[nH]c4c3C[C@@H]3C[C@]3(C)C4)nc2cc1N(C)C(=O)[C@H](C)N1CCOCC1] | 466.6 |
| | (S)-N-(5-Methoxy-2-((4aS,5aR)-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinopropanamide | |
| 37 | 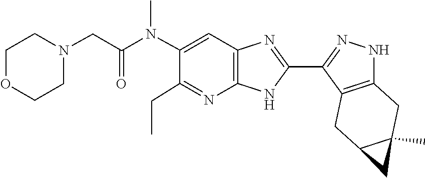 [SMILES: CCc1nc2[nH]c(-c3n[nH]c4c3C[C@@H]3C[C@]3(C)C4)nc2cc1N(C)C(=O)CN1CCOCC1] | 451.3 |
| | N-(5-Ethyl-2-((4aS,5aR)-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinoacetamide | |
| 38 | 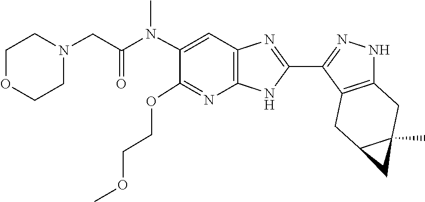 [SMILES: COCCOc1nc2[nH]c(-c3n[nH]c4c3C[C@@H]3C[C@]3(C)C4)nc2cc1N(C)C(=O)CN1CCOCC1] | 496.4 |
| | N-(5-(2-Methoxyethoxy)-2-((4aS,5aR)-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinoacetamide | |
| 39 | 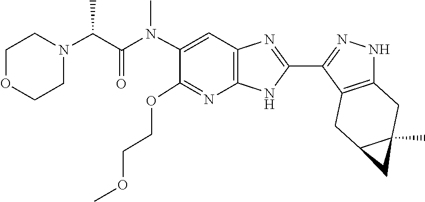 [SMILES: COCCOc1nc2[nH]c(-c3n[nH]c4c3C[C@@H]3C[C@]3(C)C4)nc2cc1N(C)C(=O)[C@@H](C)N1CCOCC1] | 510.3 |
| | (R)-N-(5-(2-Methoxyethoxy)-2-((4aS,5aR)-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinopropanamide | |
| 40 | 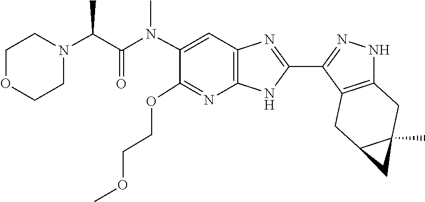 [SMILES: COCCOc1nc2[nH]c(-c3n[nH]c4c3C[C@@H]3C[C@]3(C)C4)nc2cc1N(C)C(=O)[C@H](C)N1CCOCC1] | 510.3 |
| | (S)-N-(5-(2-Methoxyethoxy)-2-((4aS,5aR)-5a-methyl-1,4,4a,5,5a,6- | |
| | hexahydrocyclopropa[f]indazol-3-yl)-3H-imidazo[4,5-b]pyridin-6-yl)-N- | |
| | methyl-2-morpholinopropanamide | |

### Examples 41 and 42

### (2S′)-2-(6-Oxa-3-azabicyclo[3.1.1]heptan-3-yl)-N-methyl-N-(2-((4aS,5aR)-5a-methyl-1,4,4a,5,5a,6-hexahydrocyclopropa[f]indazo-3-yl)-3H-imidazo[4,5-b]pyridin-6-yl)propanamide and (2R)-2-(6-oxa-3-azabicyclo[3.1.1]heptan-3-yl)-N-methyl-N-(2-((4aS,5aR)-5a-methyl-1,4,4a,5,5a,6-hexahydrocyclopropa[f]indazo-3-yl)-3H-imidazo[4,5-b]pyridin-6-yl)propanamide

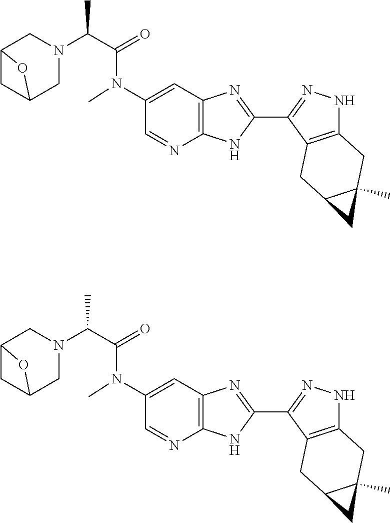 [SMILES: C[C@@H](C(=O)N(C)c1cnc2[nH]c(-c3n[nH]c4c3C[C@@H]3C[C@]3(C)C4)nc2c1)N1CC2CC(C1)O2 ; C[C@H](C(=O)N(C)c1cnc2[nH]c(-c3n[nH]c4c3C[C@@H]3C[C@]3(C)C4)nc2c1)N1CC2CC(C1)O2]

### Step 1: 2-(6-Oxa-3-azabicyclo[3.1.1]heptan-3-yl)-N-methyl-N-(2-((4aS,5aR)-5a-methyl-1-((2-(trimethylsilyl)ethoxy)methyl)-1,4,4a,5,5a,6-hexahydrocyclopropa[f]indazol-3-yl)-3H-imidazo[4,5-b]pyridin-6-yl)propenamide

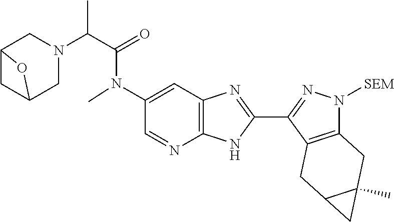 [SMILES: CC(C(=O)N(C)c1cnc2[nH]c(-c3nn(COCC[Si](C)(C)C)c4c3CC3C[C@]3(C)C4)nc2c1)N1CC2CC(C1)O2]

A mixture of Preparation 41 (200 mg, 0.39 mmol), 6-oxa-3-azabicyclo[3.1.1]heptane tosylate salt (316 mg, 1.16 mmol) and iPr₂NEt (0.34 mL, 1.94 mmol) in DMF (1.5 mL) was stirred at RT for about 16 h. The reaction mixture was then stirred at about 50° C. for about 16 h. The reaction mixture was then stirred at about 80° C. for about 16 h. The reaction mixture was cooled to RT and diluted with 3% aq. LiCl and extracted with EtOAc (2×). The organic layer was concentrated to provide the title compound. Yield: 200 mg (89%). LC-MS m/z (M+H)⁺=578.2.

### Step 2: (2S)-2-(6-Oxa-3-azabicyclo[3.1.1]heptan-3-yl)-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)propanamide and (2R)-2-(6-oxa-3-azabicyclo[3.1.1]heptan-3-yl)-N-methyl-N-(2-((4aS,5aR)-5a-methyl-1,4,4a,5,5a,6-hexahydrocyclopropa[f]indazol-3-yl)-3H-imidazo[4,5-b]pyridin-6-yl)propanamide

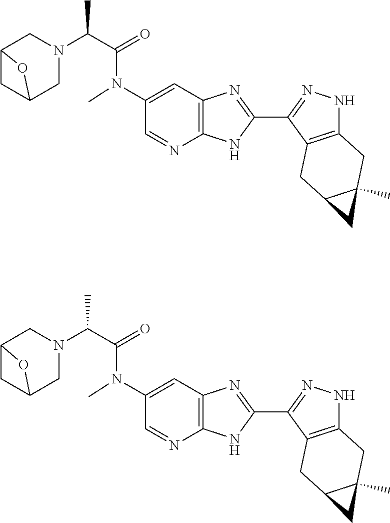 [SMILES: C[C@@H](C(=O)N(C)c1cnc2[nH]c(-c3n[nH]c4c3C[C@@H]3C[C@]3(C)C4)nc2c1)N1CC2CC(C1)O2 ; C[C@H](C(=O)N(C)c1cnc2[nH]c(-c3n[nH]c4c3C[C@@H]3C[C@]3(C)C4)nc2c1)N1CC2CC(C1)O2]

A solution of the title compound of preceding step 1 (180 mg, 0.31 mmol) in TFA (3 mL) was treated with triethylsilane (0.25 mL, 0.31 mmol) at about 15° C. and the mixture was stirred at about 15° C. for about 3 h. The reaction mixture was concentrated and the residue was taken up in sat. aq. NaHCO₃ (10 mL) and extracted with EtOAc (2×10 mL). The EtOAc extracts were combined and concentrated. The residue was combined with that from an identical reaction using 20 mg of the title compound of preceding step 1 and similar proportions of other reagents. The combined material was purified by prep-HPLC (Welch Xtimate C18 150 mm×25 mm, 5 μm; Mobile phase A: water (0.05% conc. NH₄OH); Mobile phase B: MeCN; 21-41% B gradient; 11 min, 25 mL/min) to provide the title compounds as a mixture of enantiomers (40 mg). The mixture was separated by chiral SFC (Daicel Chiralcel OD 250 mm×30 mm, 10 μm; mobile phase: 35% ethanol (0.1% conc. NH₄OH) and 65% C02; flow rate 70 mL/min) to provide the title compounds (absolute stereochemistry of individual Examples not assigned).

Example 41 19 mg (14%); 1H NMR (400 MHz, CDCl₃) δ=14.68-14.46 (m, 1H), 13.53 (br s, 1H), 8.34 (d, 1H), 8.04 (d, 1H), 4.52 (br s, 1H), 4.40 (br d, 1H), 3.69 (br d, 1H), 3.51 (q, 1H), 3.41 (s, 3H), 3.26 (td, 2H), 3.11-2.88 (m, 5H), 2.53 (dd, 1H), 2.29 (d, 1H), 1.36 (s, 3H), 1.28 (d, 3H), 1.26-1.19 (m, 1H), 0.48 (dd, 1H), 0.37 (t, 1H); LC-MS m/z (M+H)⁺=448.2.

Example 42: 21 mg (15%); ¹H NMR (400 MHz, CDCl₃) δ=14.64-14.43 (m, 1H), 13.64-13.42 (m, 1H), 8.35 (d, 1H), 8.04 (d, 1H), 4.52 (br s, 1H), 4.40 (br s, 1H), 3.68 (br d, 1H), 3.50 (q, 1H), 3.41 (s, 1H), 3.44-3.37 (m, 1H), 3.31-3.19 (m, 2H), 3.12-2.88 (m, 5H), 2.56 (dd, 1H), 2.29 (d, 1H), 1.36 (s, 3H), 1.28 (d, 3H), 1.26-1.20 (m, 1H), 0.48 (dd, 1H), 0.35 (t, 1H); LC-MS m/z (M+H)⁺=448.5.

Examples 43 and 44 were prepared in a similar manner to Examples 41 and 42, where observed LC-MS m/z is shown in the table below as [M+H]⁺.

| Ex | Structure and Name | [M + H]+ |
|---|---|---|
| 43 | 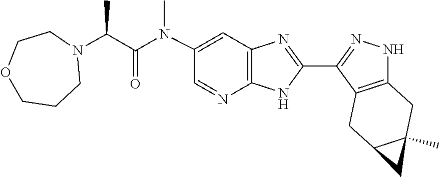 [SMILES: C[C@@H](C(=O)N(C)c1cnc2[nH]c(-c3n[nH]c4c3C[C@@H]3C[C@]3(C)C4)nc2c1)N1CCCOCC1] | 450.3 |
| | (S)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | (1,4-oxazepan-4-yl)propenamide | |
| 44 | 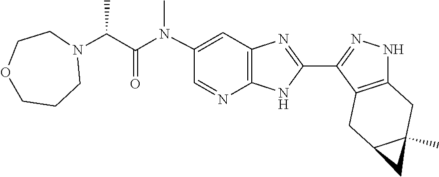 [SMILES: C[C@H](C(=O)N(C)c1cnc2[nH]c(-c3n[nH]c4c3C[C@@H]3C[C@]3(C)C4)nc2c1)N1CCCOCC1] | 450.3 |
| | (R)-N-Methyl-N-(2-((4aS,5aR)-5a-methyl-1,4,4a,5,5a,6- | |
| | hexahydrocyclopropa[f]indazol-3-yl)-3H-imidazo[4,5-b]pyridin-6-yl)-2- | |
| | (1,4-oxazepan-4-yl)propenamide | |

Examples 45 to 58 were prepared in a parallel fashion via the following protocol:

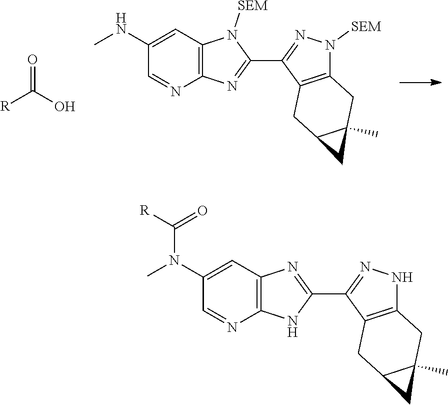

Each 2-dram vial was charged with an acid fragment (0.15 mmol) and Preparation 34 (0.5 mL of 0.2 M solution in pyridine) followed by addition of EDCl (0.5 mL of 0.3 M solution in pyridine). The vials were heated to about 80° C. and shaken for about 16 h. The solvents were removed under reduced pressure. The residue in each vial was suspended in 2:1 v/v DCM/TFA (2 mL) and treated with triethysilane (0.2 mL) and the vials were shaken at about 80° C. for about 16 h. The solvents were removed under reduced pressure. The residue in each vial was taken up in methanol (1.5 mL) and made basic with conc. NH₄OH to about pH 8. The mixtures were purified by prep-HPLC to provide the title compounds, where observed LC-MS m/z is shown in the table below as [M+H]⁺.

| Ex | Structure and Name | [M + H]+ |
|---|---|---|
| 45 | 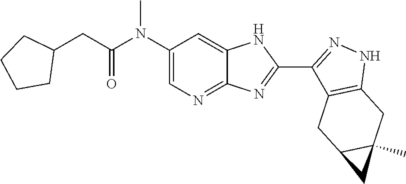 [SMILES: CN(C(=O)CC1CCCC1)c1cnc2nc(-c3n[nH]c4c3C[C@@H]3C[C@]3(C)C4)[nH]c2c1] 2-Cyclopentyl-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)acetamide | 405 |
| 46 | 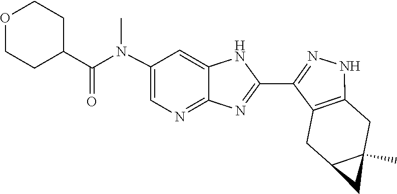 [SMILES: CN(C(=O)C1CCOCC1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-4-carboxamide | 407 |
| 47 | 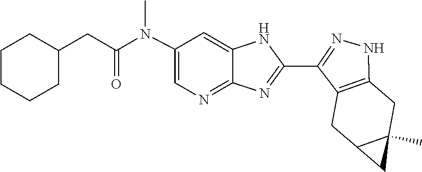 2-Cyclohexyl-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)acetamide | 419 |
| 48 | 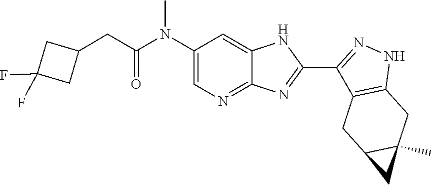 [SMILES: CN(C(=O)CC1CC(F)(F)C1)c1cnc2nc(-c3n[nH]c4c3C[C@@H]3C[C@]3(C)C4)[nH]c2c1] 2-(3,3-Difluorocyclobutyl)-N-methyl-N-(2-((4aS,5aR)-5a-methyl- 1,4,4a,5,5a,6-hexahydrocyclopropa[f]indazol-3-yl)-1H-imidazo[4,5- b]pyridin-6-yl)acetamide | 413 |
| 49 | 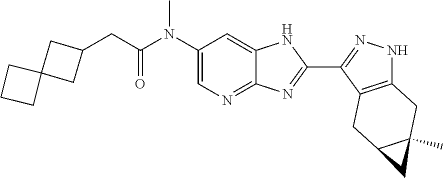 [SMILES: CN(C(=O)CC1CC2(CCC2)C1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (spiro[3.3]heptan-2-yl)acetamide | 417 |
| 50 | 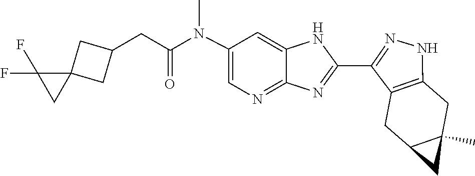 [SMILES: CN(C(=O)CC1CC2(C1)CC2(F)F)c1cnc2nc(-c3n[nH]c4c3C[C@@H]3C[C@]3(C)C4)[nH]c2c1] 2-(1,1-Difluorospiro[2.3]hexan-5-yl)-N-methyl-N-(2-((4aS,5aR)-5a- methyl-1,4,4a,5,5a,6-hexahydrocyclopropa[f]indazol-3-yl)-1H- imidazo[4,5-b]pyridin-6-yl)acetamide | 439 |
| 51 | 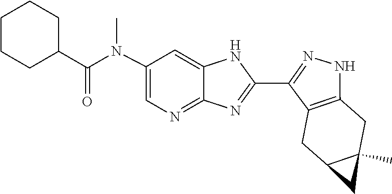 [SMILES: CN(C(=O)C1CCCCC1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclohexanecarboxamide | 405 |
| 52 | 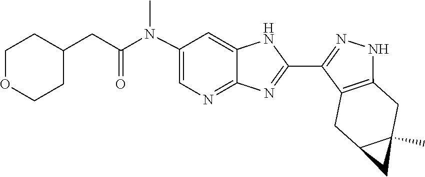 [SMILES: CN(C(=O)CC1CCOCC1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (tetrahydro-2H-pyran-4-yl)acetamide | 421 |
| 53 | 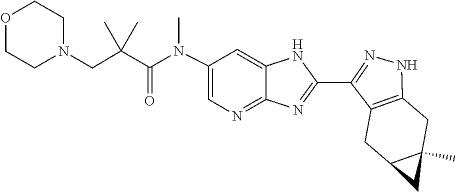 [SMILES: CN(C(=O)C(C)(C)CN1CCOCC1)c1cnc2nc(-c3n[nH]c4c3C[C@@H]3C[C@]3(C)C4)[nH]c2c1] N,2,2-Trimethyl-N-(2-((4aS,5aR)-5a-methyl-1,4,4a,5,5a,6- hexahydrocyclopropa[f]indazol-3-yl)-1H-imidazo[4,5-b]pyridin-6-yl)-3- morpholinopropanamide | 464 |
| 54 | 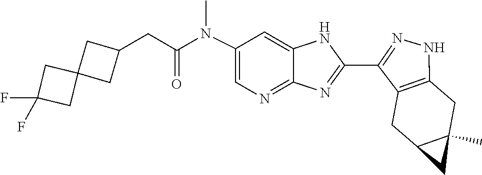 [SMILES: CN(C(=O)CC1CC2(C1)CC(F)(F)C2)c1cnc2nc(-c3n[nH]c4c3C[C@@H]3C[C@]3(C)C4)[nH]c2c1] 2-(6,6-Difluorospiro[3.3]heptan-2-yl)-N-methyl-N-(2-((4aS,5aR)-5a- methyl-1,4,4a,5,5a,6-hexahydrocyclopropa[f]indazol-3-yl)-1H- imidazo[4,5-b]pyridin-6-yl)acetamide | 453 |
| 55 | 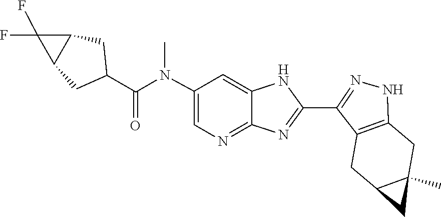 (1R,5S)-6,6-Difluoro-N-methyl-N-(2-((4aS,5aR)-5a-methyl- 1,4,4a,5,5a,6-hexahydrocyclopropa[f]indazol-3-yl)-1H-imidazo[4,5- b]pyridin-6-yl)bicyclo[3.1.0]hexane-3-carboxamide | 439 |
| 56 | 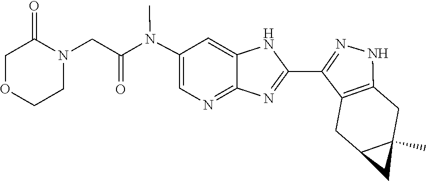 [SMILES: CN(C(=O)CN1CCOCC1=O)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (3-oxomorpholino)acetamide | 436 |
| 57 | 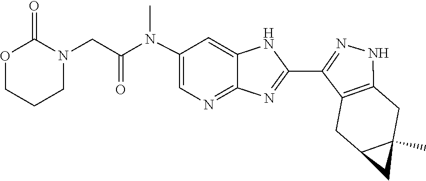 [SMILES: CN(C(=O)CN1CCCOC1=O)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (2-oxo-1,3-oxazinan-3-yl)acetamide | 436 |
| 58 | 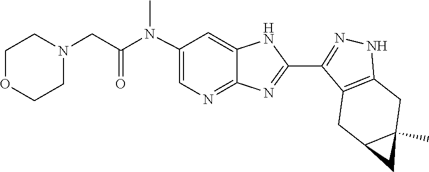 [SMILES: CN(C(=O)CN1CCOCC1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- morpholinoacetamide | 422 |

Examples 59 to 70 were prepared in a parallel fashion via the following protocol:

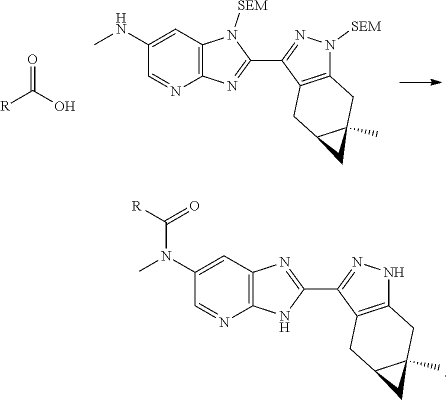

Each 2-dram vial was charged with an acid fragment (0.15 mmol) and Preparation 34 (0.2 mL of 0.5 M solution in THF) followed by addition of pyridine (10 μL) and T3P (0.2 mL of 50% w/w solution in EtOAc). The vials were heated to about 50° C. and shaken for about 16 h. The reaction mixtures were treated with sat. aq. NaHCO₃ (1 mL) and extracted with EtOAc (3×2 mL). The combined EtOAc extracts were collected and concentrated into individual vials. The residue in each vial was suspended in 2:1 DCM/TFA (2 mL) and treated with triethysilane (0.2 mL) and the vials were shaken at about 80° C. for about 16 h. The solvents were removed under reduced pressure. The residue in each vial was taken up in methanol (1.5 mL) and made basic with conc. NH₄OH to about pH 8. The mixtures were purified by prep-HPLC to provide the title compounds, where observed LC-MS m/z is shown in the table below as [M+H]⁺.

| Ex | Structure and Name | [M + H]+ |
|---|---|---|
| 59 | 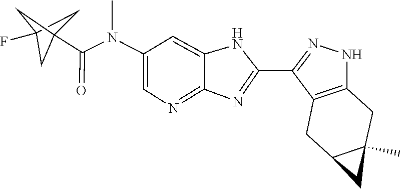 [SMILES: CN(C(=O)C12CC(F)(C1)C2)c1cnc2nc(-c3n[nH]c4c3C[C@@H]3C[C@]3(C)C4)[nH]c2c1] (3-Fluoro-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)bicyclo[1.1.1]pentane-1-carboxamide | 407 |
| 60 | 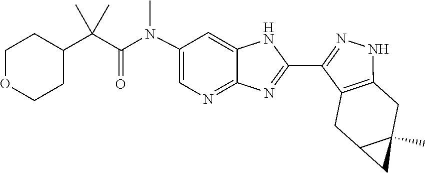 N,2-Dimethyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (tetrahydro-2H-pyran-4-yl)propenamide | 449 |
| 61 | 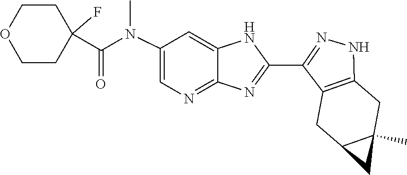 [SMILES: CN(C(=O)C1(F)CCOCC1)c1cnc2nc(-c3n[nH]c4c3C[C@@H]3C[C@]3(C)C4)[nH]c2c1] 4-Fluoro-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-4-carboxamide | 425 |
| 62 | 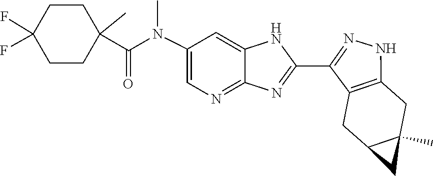 [SMILES: CN(C(=O)C1(C)CCC(F)(F)CC1)c1cnc2nc(-c3n[nH]c4c3C[C@@H]3C[C@]3(C)C4)[nH]c2c1] 4,4-Difluoro-N,1-dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclohexane-1-carboxamide | 455 |
| 63 | 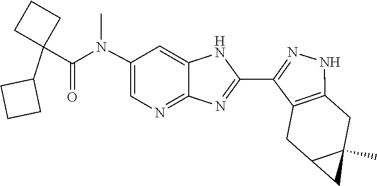 N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)- [1,1′-bi(cyclobutane)]-1-carboxamide | 431 |
| 64 | 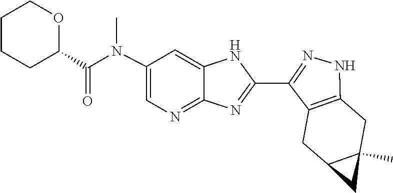 [SMILES: CN(C(=O)[C@@H]1CCCCO1)c1cnc2nc(-c3n[nH]c4c3C[C@@H]3C[C@]3(C)C4)[nH]c2c1] (S)-N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-2-carboxamide | 407 |
| 65 | 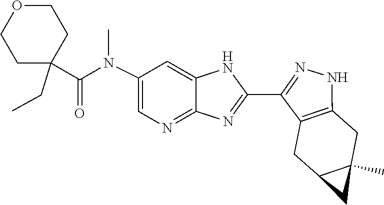 [SMILES: CCC1(C(=O)N(C)c2cnc3nc(-c4n[nH]c5c4C[C@@H]4C[C@]4(C)C5)[nH]c3c2)CCOCC1] 4-Ethyl-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-4-carboxamide | 435 |
| 66 | 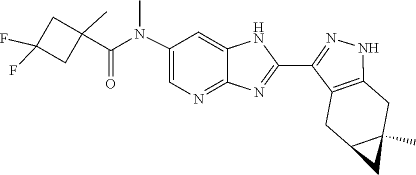 [SMILES: CN(C(=O)C1(C)CC(F)(F)C1)c1cnc2nc(-c3n[nH]c4c3C[C@@H]3C[C@]3(C)C4)[nH]c2c1] 3,3-Difluoro-N,1-dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclobutane-1-carboxamide | 427 |
| 67 | 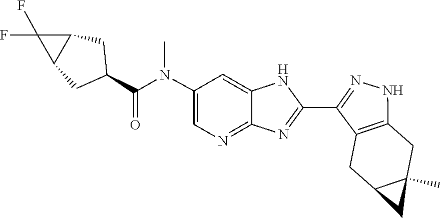 [SMILES: CN(C(=O)[C@@H]1C[C@@H]2[C@H](C1)C2(F)F)c1cnc2nc(-c3n[nH]c4c3C[C@@H]3C[C@]3(C)C4)[nH]c2c1] (1R,3s,5S)-6,6-Difluoro-N-methyl-N-(2-((4aS,5aR)-5a-methyl- 1,4,4a,5,5a,6-hexahydrocyclopropa[f]indazol-3-yl)-1H-imidazo[4,5- b]pyridin-6-yl)bicyclo[3.1.0]hexane-3-carboxamide | 439 |
| 68 | 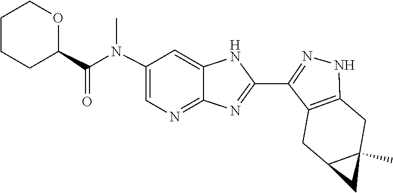 [SMILES: CN(C(=O)[C@H]1CCCCO1)c1cnc2nc(-c3n[nH]c4c3C[C@@H]3C[C@]3(C)C4)[nH]c2c1] (R)-N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-2-carboxamide | 407 |
| 69 | 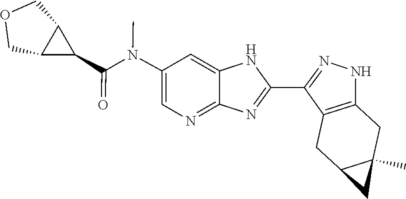 [SMILES: CN(C(=O)[C@H]1[C@@H]2COC[C@@H]21)c1cnc2nc(-c3n[nH]c4c3C[C@@H]3C[C@]3(C)C4)[nH]c2c1] (1R,5S,6r)-N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-3- oxabicyclo[3.1.0]hexane-6-carboxamide | 405 |
| 70 | 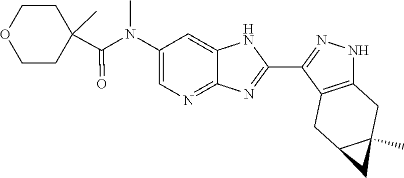 [SMILES: CN(C(=O)C1(C)CCOCC1)c1cnc2nc(-c3n[nH]c4c3C[C@@H]3C[C@]3(C)C4)[nH]c2c1] N,4-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)tetrahydro-2H-pyran-4-carboxamide | 421 |

Examples 71 to 80 were prepared in a parallel fashion via the following protocol:

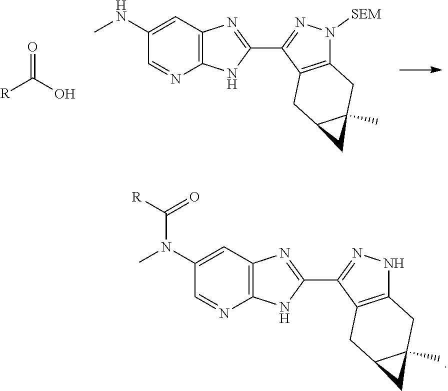

Each 2-dram vial was charged with an acid fragment (0.10 mmol) and Preparation 28 (0.15 mmol) followed by addition of pyridine (1 mL) and EDCl (0.12 mmol). The vials were heated to about 30° C. and shaken for about 16 h. The solvents were removed under reduced pressure. The residue in each vial was treated with water (1 mL) and extracted with EtOAc (3×). The combined EtOAc extracts were dried (Na₂SO₄), filtered and concentrated into vials. The residue in each vial was suspended in 5:1 DCM/TFA (1 mL) and the vials were shaken at about 30° C. for about 16 h. The solvents were removed under reduced pressure. The residue in each vial was taken up in 2:1 methanol/conc. NH₄OH (1 mL) and the vials were shaken at about 30° C. for about 16 h. The solvents were removed under reduced pressure. The crude products were purified by prep-HPLC to provide the title compounds, where observed LC-MS m/z is shown in the table below as [M+H]⁺.

| Ex | Structure and Name | [M + H]+ |
|---|---|---|
| 71 | 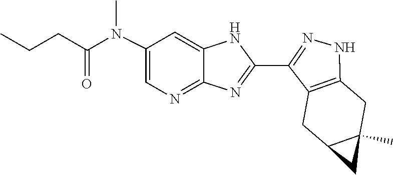 [SMILES: CCCC(=O)N(C)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)butyramide | 365 |
| 72 | 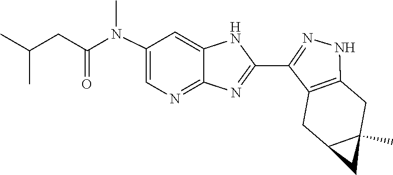 [SMILES: CC(C)CC(=O)N(C)c1cnc2nc(-c3n[nH]c4c3C[C@@H]3C[C@]3(C)C4)[nH]c2c1] N,3-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 379 |
| 73 | 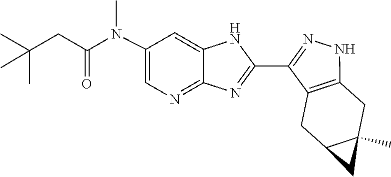 [SMILES: CN(C(=O)CC(C)(C)C)c1cnc2nc(-c3n[nH]c4c3C[C@@H]3C[C@]3(C)C4)[nH]c2c1] N,3,3-Trimethyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 393 |
| 74 | 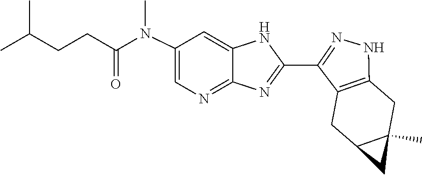 [SMILES: CC(C)CCC(=O)N(C)c1cnc2nc(-c3n[nH]c4c3C[C@@H]3C[C@]3(C)C4)[nH]c2c1] N,4-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)pentanamide | 393 |
| 75 | 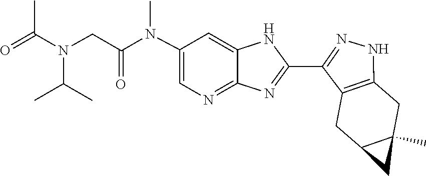 [SMILES: CC(=O)N(CC(=O)N(C)c1cnc2nc(-c3n[nH]c4c3C[C@@H]3C[C@]3(C)C4)[nH]c2c1)C(C)C] 2-(N-Isopropylacetamido)-N-methyl-N-(2-((4aS,5aR)-5a-methyl- 1,4,4a,5,5a,6-hexahydrocyclopropa[f]indazol-3-yl)-1H-imidazo[4,5- b]pyridin-6-yl)acetamide | 436 |
| 76 | 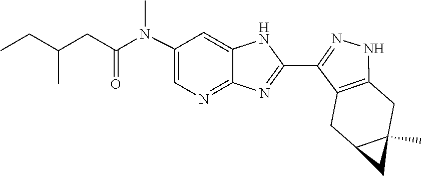 [SMILES: CCC(C)CC(=O)N(C)c1cnc2nc(-c3n[nH]c4c3C[C@@H]3C[C@]3(C)C4)[nH]c2c1] N,3-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)pentanamide | 393 |
| 77 | 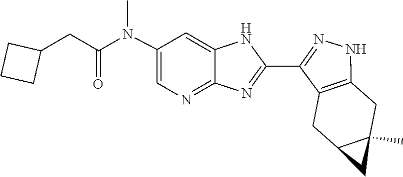 [SMILES: CN(C(=O)CC1CCC1)c1cnc2nc(-c3n[nH]c4c3C[C@@H]3C[C@]3(C)C4)[nH]c2c1] 2-Cyclobutyl-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)acetamide | 391 |
| 78 | 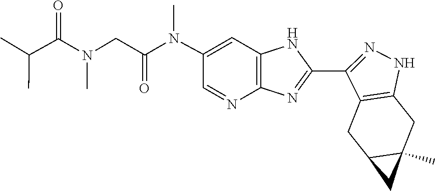 [SMILES: CC(C)C(=O)N(C)CC(=O)N(C)c1cnc2nc(-c3n[nH]c4c3C[C@@H]3C[C@]3(C)C4)[nH]c2c1] N-Methyl-N-(2-(methyl(2-((4aS,5aR)-5a-methyl-1,4,4a,5,5a,6- hexahydrocyclopropa[f]indazol-3-yl)-1H-imidazo[4,5-b]pyridin-6- yl)amino)-2-oxoethyl)isobutyramide | 436 |
| 79 | 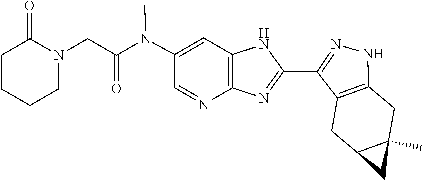 [SMILES: CN(C(=O)CN1CCCCC1=O)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (2-oxopiperidin-1-yl)acetamide | 434 |
| 80 | 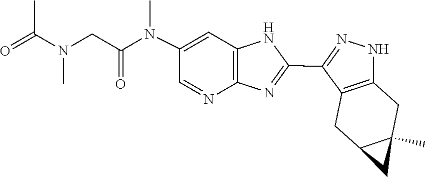 [SMILES: CC(=O)N(C)CC(=O)N(C)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6-yl)-2- (N-methylacetamido)acetamide | 408 |

Examples 81 to 89 were prepared in a parallel fashion via the following protocol:

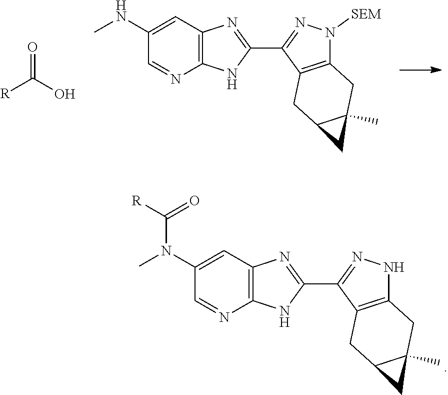

Each 2-dram vial was charged with an acid fragment (0.10 mmol) and Preparation 28 (0.15 mmol) followed by addition of pyridine (300 μL), THF (12 μL) and T₃P (0.2 mL of 50% w/w solution in EtOAc). The vials were heated to about 80° C. and shaken for about 16 h. The solvents were removed under reduced pressure. The residue in each vial was treated with water (1 mL) and extracted with EtOAc (3×). The combined EtOAc extracts were dried (Na₂SO₄), filtered and concentrated into vials. The residue in each vial was suspended in 5:1 DCM/TFA (1 mL) and the vials were shaken at about 30° C. for about 16 h. The solvents were removed under reduced pressure. The residue in each vial was taken up in 2:1 methanol/conc. NH₄OH (1 mL) and the vials were shaken at about 30° C. for about 16 h. The solvents were removed under reduced pressure. The crude products were purified by prep-HPLC to provide the title compounds, where observed LC-MS m/z is shown in the table below as [M+H]⁺.

| Ex | Structure and Name | [M + H]+ |
|---|---|---|
| 81 | 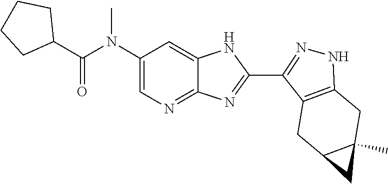 [SMILES: CN(C(=O)C1CCCC1)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclopentanecarboxamide | 391 |
| 82 | 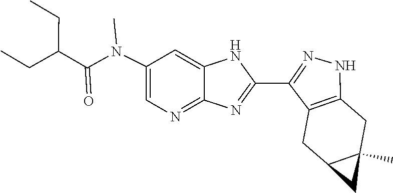 [SMILES: CCC(CC)C(=O)N(C)c1cnc2nc(-c3n[nH]c4c3C[C@@H]3C[C@]3(C)C4)[nH]c2c1] 2-Ethyl-N-methyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 393 |
| 83 | 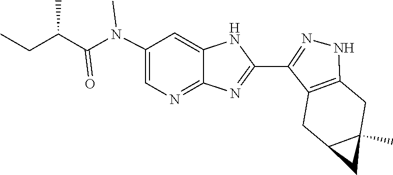 [SMILES: CC[C@H](C)C(=O)N(C)c1cnc2nc(-c3n[nH]c4c3C[C@@H]3C[C@]3(C)C4)[nH]c2c1] (S)-N,2-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 379 |
| 84 | 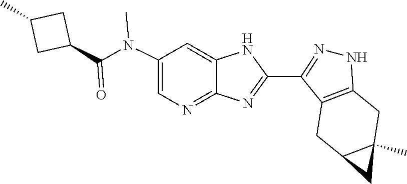 (1r,3S)-N,3-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclobutane-1-carboxamide | 391 |
| 85 | 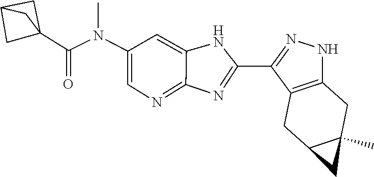 [SMILES: CN(C(=O)C12CC(C1)C2)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)bicyclo[1.1.1]pentane-1-carboxamide | 389 |
| 86 | 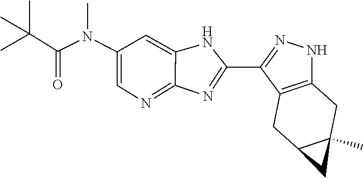 [SMILES: CN(C(=O)C(C)(C)C)c1cnc2nc(-c3n[nH]c4c3C[C@@H]3C[C@]3(C)C4)[nH]c2c1] N-Methyl-N-(2-((4aS,5aR)-5a-methyl-1,4,4a,5,5a,6- hexahydrocyclopropa[f]indazol-3-yl)-1H-imidazo[4,5-b]pyridin-6- yl)pivalamide | 379 |
| 87 | 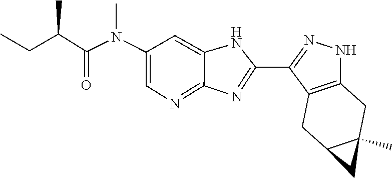 [SMILES: CC[C@@H](C)C(=O)N(C)c1cnc2nc(-c3n[nH]c4c3C[C@@H]3C[C@]3(C)C4)[nH]c2c1] (R)-N,2-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 379 |
| 88 | 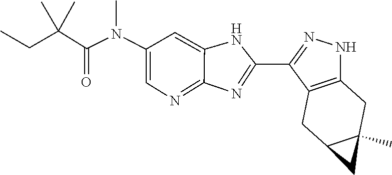 [SMILES: CCC(C)(C)C(=O)N(C)c1cnc2nc(-c3n[nH]c4c3C[C@@H]3C[C@]3(C)C4)[nH]c2c1] N,2,2-Trimethyl-N-(2-((4aS,5aR)-5a-methyl-1,4,4a,5,5a,6- hexahydrocyclopropa[f]indazol-3-yl)-1H-imidazo[4,5-b]pyridin-6- yl)butanamide | 393 |
| 89 | 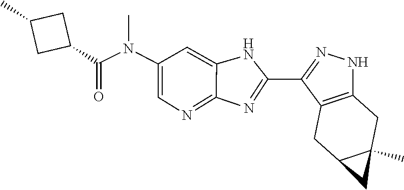 (1s,3R)-N,3-Dimethyl-N-(2-((4aS,5aR)-5a-methyl-1,4,4a,5,5a,6- hexahydrocyclopropa[f]indazol-3-yl)-1H-imidazo[4,5-b]pyridin-6- yl)cyclobutane-1-carboxamide | 391 |

Examples 90 to 114 were prepared in a parallel fashion via the following protocol:

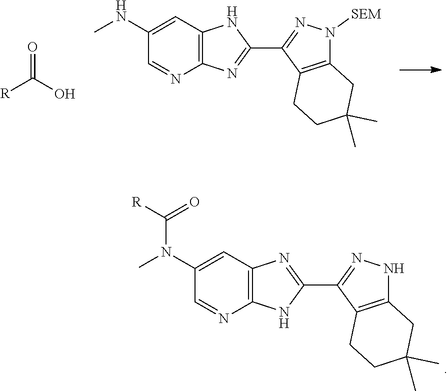

Solution A: Preparation 35 (2.10 g) and methylimidazole (850 mg) in DCM (42 mL).

Solution B: TCFH (2.1 g) in DCM (42 mL).

To a 2-dram vial charged with an acid fragment (0.15 mmol) was added Solution A (1 mL) and Solution B (1 mL) and the mixture was shaken at RT for about 16 h. Water (2 ml) and 2CM (2 mL) were added and the CM layer was concentrated. The residue was taken up in 1,2-dichloroethane (1 ml) and treated with TFA (1 ml) and shaken at about 50° C. for about 16 h. The solvents were removed under reduced pressure. The crude products were purified by prep-HPLC to provide the title compounds, where observed LC-MS m/z is shown in the table below as [M+H]⁺.

| Ex | Structure and Name | [M + H]+ |
|---|---|---|
| 90 | 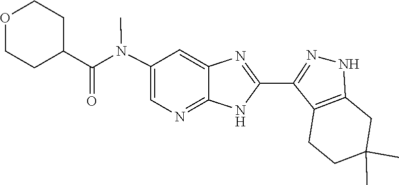 [SMILES: CN(C(=O)C1CCOCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methyltetrahydro-2H-pyran-4- carboxamide | 409.5 |
| 91 | 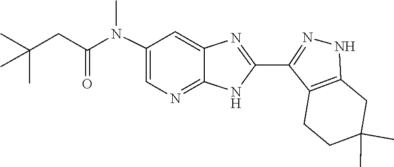 [SMILES: CN(C(=O)CC(C)(C)C)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N,3,3-trimethylbutanamide | 395.5 |
| 92 | 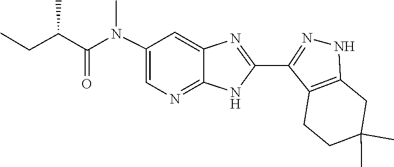 [SMILES: CC[C@H](C)C(=O)N(C)c1cnc2[nH]c(-c3n[nH]c4c3CCC(C)(C)C4)nc2c1] (S)-N-(2-(6,6-Dimethyl-4,5,6,7-tetrahydro-1H-indazol-3-yl)-3H- imidazo[4,5-b]pyridin-6-yl)-N,2-dimethylbutanamide | 381.5 |
| 93 | 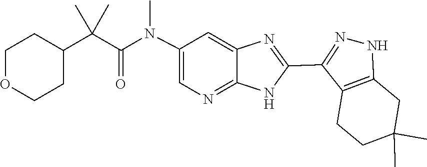 [SMILES: CN(C(=O)C(C)(C)C1CCOCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N,2-dimethyl-2-(tetrahydro-2H-pyran-4- yl)propanamide | 451.5 |
| 94 | 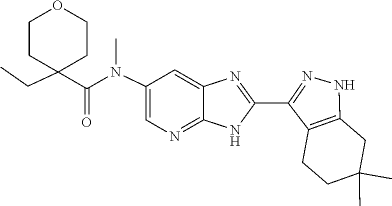 [SMILES: CCC1(C(=O)N(C)c2cnc3[nH]c(-c4n[nH]c5c4CCC(C)(C)C5)nc3c2)CCOCC1] N-(2-(6,6-Dimethyl-4,5,6,7-tetrahydro-1H-indazol-3-yl)-3H- imidazo[4,5-b]pyridin-6-yl)-4-ethyl-N-methyltetrahydro-2H-pyran-4- carboxamide | 437.5 |
| 95 | 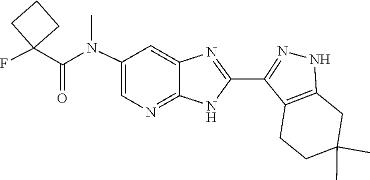 [SMILES: CN(C(=O)C1(F)CCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-1-fluoro-N-methylcyclobutane-1- carboxamide | 397.4 |
| 96 | 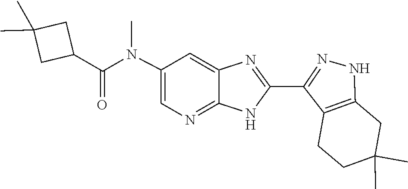 N-(2-(6,6-Dimethyl-4,5,6,7-tetrahydro-1H-indazol-3-yl)-3H- imidazo[4,5-b]pyridin-6-yl)-N,3,3-trimethylcyclobutane-1-carboxamide | 407.5 |
| 97 | 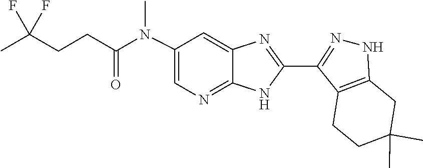 [SMILES: CN(C(=O)CCC(C)(F)F)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-4,4-difluoro-N-methylpentanamide | 417.4 |
| 98 | 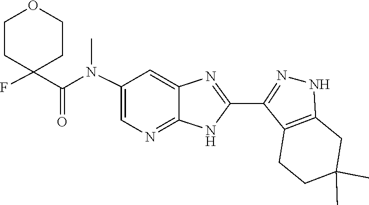 [SMILES: CN(C(=O)C1(F)CCOCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-4-fluoro-N-methyltetrahydro-2H-pyran-4- carboxamide | 427.5 |
| 99 | 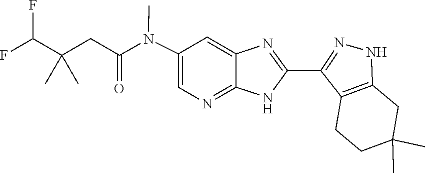 [SMILES: CN(C(=O)CC(C)(C)C(F)F)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-4,4-difluoro-N,3,3-trimethylbutanamide | 431.5 |
| 100 | 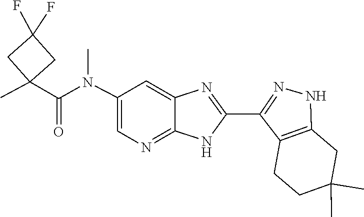 N-(2-(6,6-Dimethyl-4,5,6,7-tetrahydro-1H-indazol-3-yl)-3H- imidazo[4,5-b]pyridin-6-yl)-3,3-difluoro-N,1-dimethylcyclobutane-1- carboxamide | 429.4 |
| 101 | 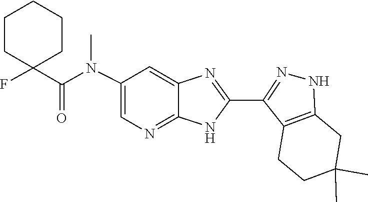 [SMILES: CN(C(=O)C1(F)CCCCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-1-fluoro-N-methylcyclohexane-1- carboxamide | 425.5 |
| 102 | 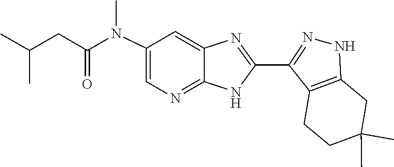 [SMILES: CC(C)CC(=O)N(C)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N,3-dimethylbutanamide | 381.6 |
| 103 | 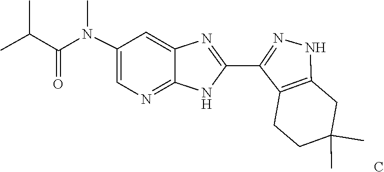 N-(2-(6,6-Dimethyl-4,5,6,7-tetrahydro-1H-indazol-3-yl)-3H- imidazo[4,5-b]pyridin-6-yl)-N-methylisobutyramide | 367.6 |
| 104 | 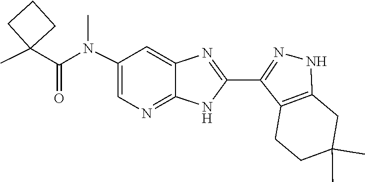 N-(2-(6,6-Dimethyl-4,5,6,7-tetrahydro-1H-indazol-3-yl)-3H- imidazo[4,5-b]pyridin-6-yl)-N,1-dimethylcyclobutane-1-carboxamide | 393.6 |
| 105 | 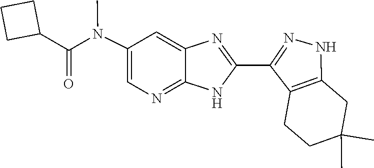 [SMILES: CN(C(=O)C1CCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methylcyclobutanecarboxamide | 379.6 |
| 106 | 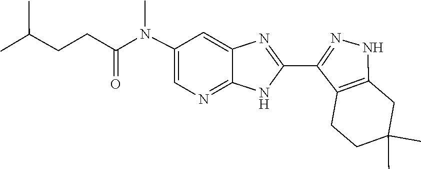 [SMILES: CC(C)CCC(=O)N(C)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N,4-dimethylpentanamide | 395.6 |
| 107 | 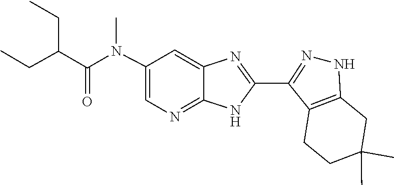 [SMILES: CCC(CC)C(=O)N(C)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-2-ethyl-N-methylbutanamide | 395.5 |
| 108 | 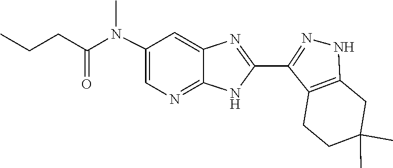 [SMILES: CCCC(=O)N(C)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methylbutyramide | 367.5 |
| 109 | 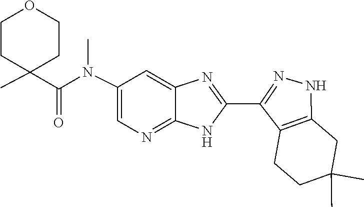 N-(2-(6,6-Dimethyl-4,5,6,7-tetrahydro-1H-indazol-3-yl)-3H- imidazo[4,5-b]pyridin-6-yl)-N,4-dimethyltetrahydro-2H-pyran-4- carboxamide | 423.5 |
| 110 | 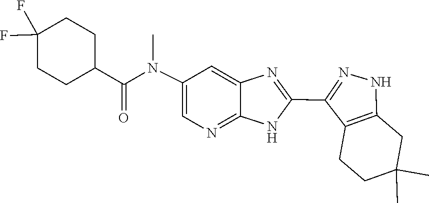 [SMILES: CN(C(=O)C1CCC(F)(F)CC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-4,4-difluoro-N-methylcyclohexane-1- carboxamide | 443.5 |
| 111 | 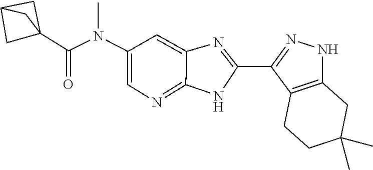 [SMILES: CN(C(=O)C12CC(C1)C2)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methylbicyclo[1.1.1]pentane-1- carboxamide | 391.4 |
| 112 | 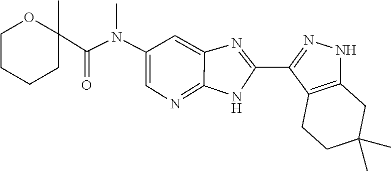 [SMILES: CN(C(=O)C1(C)CCCCO1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N,2-dimethyltetrahydro-2H-pyran-2- carboxamide | 423.5 |
| 113 | 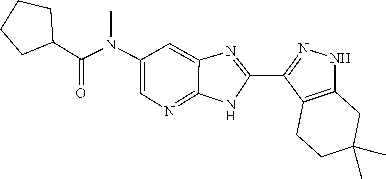 [SMILES: CN(C(=O)C1CCCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methylcyclopentanecarboxamide | 393.6 |
| 114 | 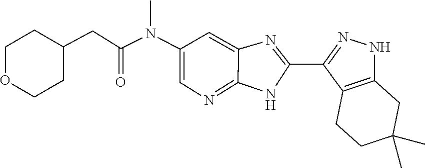 [SMILES: CN(C(=O)CC1CCOCC1)c1cnc2[nH]c(-c3n[nH]c4c3CCC(C)(C)C4)nc2c1] N-(2-(6,6-Dimethyl-4,5,6,7-tetrahydro-1H-indazol-3-yl)-3H- imidazo[4,5-b]pyridin-6-yl)-N-methyl-2-(tetrahydro-2H-pyran-4- yl)acetamide | 423.6 |

Examples 115 to 120 were prepared in a parallel fashion via the following protocol:

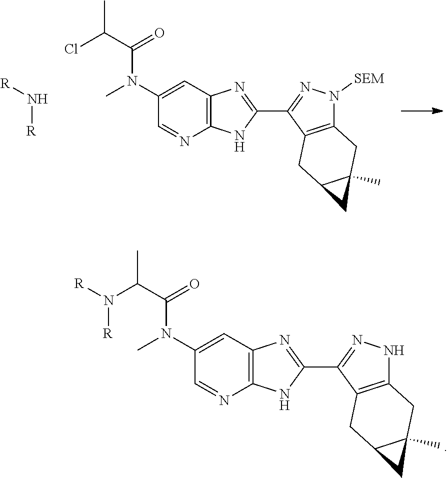

To a 2-dram vial charged with an amine fragment (0.3 mmol) was added Preparation 41 (103 mg, 0.2 mmol) as a solution in DMAC (1 mL) and iPr₂NEt (0.1 mL, 0.56 mmol). The mixture was shaken at about 100° C. for about 48 h. The reaction mixtures were cooled to RT and concentrated. The residue was taken up in TFA (1 mL) and treated with triethylsilane (0.1 mL). The reaction mixture was shaken at about 50° C. for about 16 h. The solvents were removed under reduced pressure. The crude products were purified by prep-HPLC to provide the title compounds, where observed LC-MS m/z is shown in the table below as [M+H]⁺.

| Ex | Structure and Name | [M + H]+ |
|---|---|---|
| 115 | 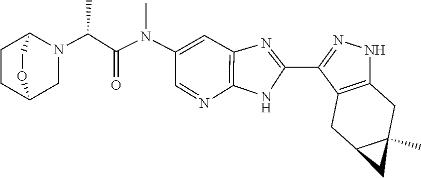 [SMILES: C[C@H](C(=O)N(C)c1cnc2[nH]c(-c3n[nH]c4c3C[C@@H]3C[C@]3(C)C4)nc2c1)N1C[C@H]2CC[C@@H]1CO2] (R)-2-((1R,4R)-2-Oxa-5-azabicyclo[2.2.2]octan-5-yl)-N-methyl-N-(2- ((4aS,5aR)-5a-methyl-1,4,4a,5,5a,6-hexahydrocyclopropa[f]indazol-3- yl)-3H-imidazo[4,5-b]pyridin-6-yl)propenamide | 462.5 |
| 116 | 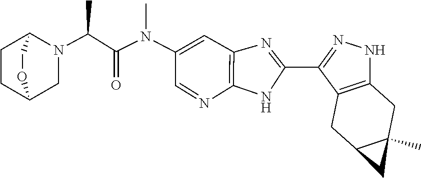 [SMILES: C[C@@H](C(=O)N(C)c1cnc2[nH]c(-c3n[nH]c4c3C[C@@H]3C[C@]3(C)C4)nc2c1)N1C[C@H]2CC[C@@H]1CO2] (S)-2-((1R,4R)-2-Oxa-5-azabicyclo[2.2.2]octan-5-yl)-N-methyl-N-(2- ((4aS,5aR)-5a-methyl-1,4,4a,5,5a,6-hexahydrocyclopropa[f]indazol-3- yl)-3H-imidazo[4,5-b]pyridin-6-yl)propanamide | 462.5 |
| 117 | 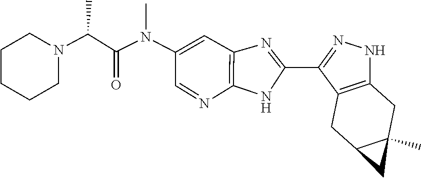 [SMILES: C[C@H](C(=O)N(C)c1cnc2[nH]c(-c3n[nH]c4c3C[C@@H]3C[C@]3(C)C4)nc2c1)N1CCCCC1] (R)-N-Methyl-N-(2-((4aS,5aR)-5a-methyl-1,4,4a,5,5a,6- hexahydrocyclopropa[f]indazol-3-yl)-3H-imidazo[4,5-b]pyridin-6-yl)-2- (piperidin-1-yl)propanamide | 434.5 |
| 118 | 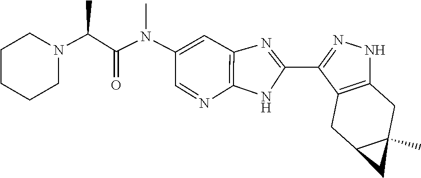 [SMILES: C[C@@H](C(=O)N(C)c1cnc2[nH]c(-c3n[nH]c4c3C[C@@H]3C[C@]3(C)C4)nc2c1)N1CCCCC1] (S)-N-Methyl-N-(2-((4aS,5aR)-5a-methyl-1,4,4a,5,5a,6- hexahydrocyclopropa[f]indazol-3-yl)-3H-imidazo[4,5-b]pyridin-6-yl)-2- (piperidin-1-yl)propanamide | 434.5 |
| 119 | 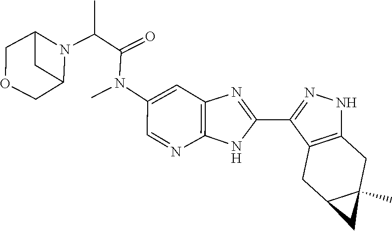 2-(3-Oxa-6-azabicyclo[3.1.1]heptan-6-yl)-N-methyl-N-(2-((4aS,5aR)- 5a-methyl-1,4,4a,5,5a,6-hexahydrocyclopropa[f]indazol-3-yl)-3H- imidazo[4,5-b]pyridin-6-yl)propanamide | 448.6 |
| 120 | 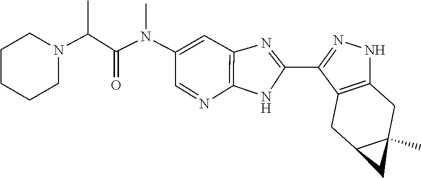 [SMILES: CC(C(=O)N(C)c1cnc2[nH]c(-c3n[nH]c4c3C[C@@H]3C[C@]3(C)C4)nc2c1)N1CCCCC1] N-Methyl-N-(2-((4aS,5aR)-5a-methyl-1,4,4a,5,5a,6- hexahydrocyclopropa[f]indazol-3-yl)-3H-imidazo[4,5-b]pyridin-6-yl)-2 | 434.5 |

### Biological Assays

In Vitro Studies

IL-2-Inducible T-Cell Kinase (ITK) Activity, IC₅₀ (uM)

ITK activity was determined by measuring the effect of a test compound in an ITK enzyme assay.

1.0 M HEPES Buffer pH 7.5 solution was prepared as follows: 238.3 g HEPES free acid (Sigma) and 800 mL of water were combined, and the mixture was stirred until complete dissolution. The pH was adjusted to 7.5 via titration with 5N NaOH and the volume adjusted to 1000 mL. The solution was filtered and sterilized.

ITK assay buffer was prepared as follows: 50 mL of HPLC-grade water was treated with 2 mL of 1.0 M HEPES Buffer, 500 μL of 2% Gelatin (Sigma), 1.0 mL of aqueous MgCl₂ solution (1.0 M), and 1.0 mL of aqueous glutathione solution (0.5 M), and the solution was mixed. The solution was brought to 99 mL in a graduated cylinder by addition of water and sterilized through a 0.2 μm filter. 0.1 mL of Brij-35™ Surfact-Amps™ Detergent Solution (10% w/v aqueous solution, ThermoFisher) and 1.0 mL of ATP (Teknova,100 mM) were added and the solution was mixed.

Preparation of 1.33× ITK enzyme solution was as follows: 49.99 mL of ITK assay buffer was treated with 4.1 μL of ITK enzyme (ITK FL (N-Flag and C-His tagged, −72 kDa) Lake Pharma, 0.25 mg/ml in a buffer containing 25 mM Tris pH 7.8, 150 mM NaCl, 10% glycerol and 2 mM TCEP) and the mixture was gently agitated. The resulting solution was stored on ice. 30 Minutes prior to use, the enzyme solution was removed from ice and equilibrated to RT by incubation in a RT water bath.

Preparation of 4× ITK substrate solution was as follows: 50 mL of ITK assay buffer was treated with 100 μL of BTK peptide (China Peptide Company, 2 mM stock solution in DMSO). The tube was capped, mixed by gently inverting the tube, and then stored on ice. 30 Minutes prior to use, the substrate solution was removed from ice and equilibrated to RT by incubation in a RT water bath.

At the time of assay, 7.5 μL of the 1.33× ITK enzyme solution was added to plate wells containing 0.1 μL of varying concentrations of test compound in DMSO. The plate was incubated 30 min at RT. The plate wells were each treated with 2.5 uL of the 4× ITK substrate solution and the plate was sealed (TopSeal™, Perkin Elmer). The plate was spun at 1000 rpm for 30 sec and then incubated for 60 min at RT. The seal was removed, and each well was treated with 10 μL of Stop/Detect Buffer (20 mM HEPES pH 7.5, 0.01% gelatin, 1 nM LANCE PT66 (Perkin Elmer), 16.5 μg/ml Surelight APC (Perkin Elmer), 10 mM EDTA, 250 mM NaCl). The plate was again covered and was spun at 1000 rpm for seconds. The plate was allowed to incubate overnight at RT and in a closed carrier to reduce dehydration. The seal was removed, and the fluorescence was read with a plate reader with an excitation wavelength of 665 nm and an emission wavelength of 615 nm. The concentrations and resulting effect values for the tested compound were plotted and the concentration of compound required for 50% effect (IC₅₀) was determined with the four-parameter logistic dose response equation.

IC₅₀ (uM) values for compounds of the invention are presented in the Table that follows.

IL-2-Inhibition Activity, IC₅₀ (uM)

IL-2 inhibition activity in supernatants from activated CD4+ human T-cells was determined by measuring the effect of a test compound on the activity using the cisbio HTRF™ technology.

Human CD4+ T cells were activated with CD3/CD28 for 3 days and expanded for an additional 4-6 days (7 to 9 days total). On day 0, frozen CD4+ T cells were thawed, treated with CD3/CD28 Dynabeads, and incubated at 37° C./5% CO₂. On day 3, the beads were removed, and the cells were diluted to 5×10⁵ cells/cm², placed in G-Rex10 flask, and incubated at 37° C./5% CO₂. On day 7 to day 9 the cells were removed from the G-Rex flasks, counted and diluted back to 1×10⁶ cells/ml in standard tissue culture flask.

The expanded CD4+ T-cells were centrifuged at 300×g for 10 minutes and resuspended to 0.5 million cells per ml (30,000 cells/well). 60 μl of CD4+ T cells were added per well to a 384 well plate containing 0.1 μL of varying concentrations of test compound in DMSO. The plates were incubated for 15 min at 37° C./5% CO₂. 20 μl of diluted ImmunoCult™ (STEMCELL Technologies, 1:12.5 in T cell assay media) were added to all wells of the plate (1:50 final assay concentration). The plates were incubated for an additional 20 to 24 hrs at 37° C./5% CO₂. The plates were centrifuged at 300×g for 10 minutes. 16 μL of supernatant was removed and combined with 4 μl of IL-2 HTRF Abs. (cisbio kit). Plates were incubated for 3 hours at RT and read with an EnVision plate reader at 665 nm and 615 nm wavelengths. The concentrations and resulting effect values for the tested compound were plotted and the concentration of compound required for 50% effect (IC₅₀) was determined with the four-parameter logistic dose response equation.

IC₅₀ (uM) values for compounds of the invention are presented in the Table that follows.

Tropomyosin Receptor Kinase a (TRKA) Activity, % Inhibition

Assays to determine TRKA activity are known in the art; e.g. see those described in:
Skerratt S E, et al. J. Med. Chem. (2016), 59(22):10084-10099 PMID: 27766865. DOI: 10.1021/acs.jmedchem.6b00850
Bagal S K, et al.. J. Med. Chem. (2018), 61(15):6779-6800 PMID: 29944371. DOI: 10.1021/acs.jmedchem.8b00633

TRKA, also known as neurotrophic tyrosine kinase receptor type 1 (NTKR1) activity was determined by measuring the effect of a test compound on the activity against the NTRK1 enzyme using the ThermoFisher Z′-LYTE Assay fluorescence-based coupled enzyme format (www.thermofisher.com/selectscreen). Test compounds were screened at a fixed concentration of 1 uM and the % inhibition was determined compared to controls at a fixed ATP concentration of 1 mM. The resulting effect value for the tested compound was compared to the assay controls to determine the % inhibition (%).

% Inhibition (%) values for compounds of the invention are presented in the Table that follows.

TABLE
In Vitro Study Data

| | ITK IC50 | ITK | IL-2 IC50 | IL-2 | TRKA % | TRKA |
|---|---|---|---|---|---|---|
| Ex # | (uM)¹ | count (n) | (uM)² | count (n) | inhibition (%)³ | count (n) |
| 1 | 0.009 | 4 | 0.067 | 4 | 96 | 2 |
| 2 | 0.014 | 12 | 0.092 | 15 | 108 | 2 |
| 3 | 0.038 | 3 | 0.320 | 3 | NT | |
| 4 | 0.038 | 2 | 0.205 | 3 | NT | |
| 5 | 0.014 | 2 | 0.375 | 3 | NT | |
| 6 | 0.026 | 2 | 0.343 | 3 | NT | |
| 7 | 0.070 | 2 | 0.398 | 2 | NT | |
| 8 | 0.117 | 2 | 0.279 | 3 | NT | |
| 9 | 0.024 | 1 | 0.147 | 1 | NT | |
| 10 | 0.091 | 3 | 0.338 | 3 | 98 | 2 |
| 11 | 0.072 | 3 | 0.534 | 2 | NT | |
| 12 | 0.076 | 3 | 0.447 | 3 | NT | |
| 13 | 0.102 | 3 | 0.559 | 3 | NT | |
| 14 | 0.147 | 6 | 0.157 | 3 | 92 | 2 |
| 15 | 0.083 | 4 | 1.234 | 2 | NT | |
| 16 | 0.747 | 2 | 2.110 | 2 | NT | |
| 17 | 0.041 | 4 | 0.307 | 3 | 96 | 4 |
| 18 | 0.028 | 4 | 0.162 | 3 | 97 | 2 |
| 19 | 0.017 | 4 | 0.153 | 2 | 98 | 2 |
| 20 | 0.231 | 2 | 1.205 | 2 | NT | |
| 21 | 0.038 | 2 | 0.193 | 3 | NT | |
| 22 | 0.085 | 3 | 0.471 | 3 | 94 | 2 |
| 23 | 1.332 | 2 | 1.851 | 1 | 54 | 2 |
| 24 | 0.003 | 10 | 0.034 | 6 | 95 | 2 |
| 25 | 0.130 | 2 | 0.709 | 2 | NT | |
| 26 | 0.136 | 3 | 0.631 | 2 | 91 | 2 |
| 27 | 0.062 | 1 | NT | | NT | |
| 28 | 0.023 | 36 | 0.121 | 21 | 91 | 2 |
| 29 | 2.850 | 2 | 2.527 | 2 | 69 | 2 |
| 30 | 0.008 | 3 | 0.079 | 3 | 97 | 2 |
| 31 | 0.025 | 2 | 0.415 | 3 | 95 | 2 |
| 32 | 0.017 | 3 | 0.167 | 2 | 94 | 2 |
| 33 | 2.004 | 1 | 0.315 | 1 | NT | |
| 34 | 0.061 | 3 | 0.481 | 4 | NT | |
| 35 | 0.143 | 3 | 0.462 | 3 | NT | |
| 36 | 0.047 | 3 | 0.172 | 3 | 95 | 4 |
| 37 | 0.147 | 3 | 0.815 | 3 | NT | |
| 38 | 0.152 | 4 | 0.464 | 3 | NT | |
| 39 | 0.110 | 2 | 0.461 | 2 | NT | |
| 40 | 0.028 | 4 | 0.182 | 3 | 97 | 2 |
| 41 | 0.116 | 1 | 0.600 | 2 | NT | |
| 42 | 0.021 | 2 | 0.143 | 2 | NT | |
| 43 | 0.134 | 2 | 0.351 | 2 | NT | |
| 44 | 0.101 | 1 | 0.226 | 2 | NT | |
| 45 | 0.025 | 2 | 0.117 | 3 | NT | |
| 46 | 0.123 | 2 | 0.294 | 2 | NT | |
| 47 | 0.044 | 2 | 0.189 | 3 | 95 | 2 |
| 48 | 0.066 | 2 | 0.326 | 3 | NT | |
| 49 | 0.025 | 2 | 0.157 | 3 | NT | |
| 50 | 0.033 | 2 | 0.171 | 3 | NT | |
| 51 | 0.037 | 2 | 0.108 | 3 | NT | |
| 52 | 0.051 | 3 | 0.189 | 3 | NT | |
| 53 | 0.088 | 2 | 0.304 | 2 | NT | |
| 54 | 0.062 | 2 | 0.131 | 2 | NT | |
| 55 | 0.020 | 2 | 0.093 | 3 | NT | |
| 56 | 0.054 | 3 | 0.611 | 2 | NT | |
| 57 | 0.036 | 2 | 0.974 | 3 | 106 | 2 |
| 58 | 0.102 | 2 | 0.380 | 2 | 108 | 2 |
| 59 | 0.109 | 1 | 0.370 | 2 | NT | |
| 60 | 0.063 | 2 | 0.182 | 3 | NT | |
| 61 | 0.023 | 2 | 0.088 | 3 | 95 | 2 |
| 62 | 0.141 | 2 | 0.402 | 3 | NT | |
| 63 | 0.040 | 2 | 0.338 | 3 | NT | |
| 64 | 0.103 | 2 | 0.208 | 3 | NT | |
| 65 | 0.150 | 2 | 0.568 | 2 | NT | |
| 66 | 0.092 | 2 | 0.292 | 3 | NT | |
| 67 | 0.012 | 2 | 0.059 | 3 | NT | |
| 68 | 0.107 | 2 | 0.177 | 3 | NT | |
| 69 | 0.292 | 1 | 1.161 | 2 | NT | |
| 70 | 0.093 | 2 | 0.462 | 2 | NT | |
| 71 | 0.042 | 3 | 0.313 | 3 | NT | |
| 72 | 0.039 | 3 | 0.248 | 3 | NT | |
| 73 | 0.057 | 2 | 0.498 | 1 | NT | |
| 74 | 0.050 | 3 | 0.311 | 3 | NT | |
| 75 | 0.057 | 3 | 0.862 | 2 | NT | |
| 76 | 0.051 | 2 | 0.389 | 2 | NT | |
| 77 | 0.027 | 3 | 0.247 | 2 | NT | |
| 78 | 0.035 | 3 | 0.552 | 2 | NT | |
| 79 | 0.043 | 3 | 1.590 | 2 | NT | |
| 80 | 0.054 | 3 | 1.253 | 3 | 96 | 2 |
| 81 | 0.021 | 3 | 0.182 | 3 | NT | |
| 82 | 0.020 | 3 | 0.176 | 3 | NT | |
| 83 | 0.047 | 3 | 0.371 | 2 | NT | |
| 84 | 0.031 | 3 | 0.288 | 3 | 96 | 4 |
| 85 | 0.091 | 3 | 1.219 | 2 | NT | |
| 86 | 0.100 | 2 | 0.466 | 5 | 95 | 2 |
| 87 | 0.026 | 3 | 0.246 | 2 | NT | |
| 88 | 0.118 | 2 | 0.808 | 2 | NT | |
| 89 | 0.027 | 2 | 0.324 | 2 | NT | |
| 90 | 0.545 | 2 | 0.892 | 1 | NT | |
| 91 | 0.144 | 3 | 0.384 | 3 | NT | |
| 92 | 0.698 | 3 | >10.000 | 1 | NT | |
| 93 | 0.333 | 2 | NT | | NT | |
| 94 | 0.307 | 3 | 3.170 | 2 | NT | |
| 95 | 0.064 | 3 | 0.266 | 3 | 93 | 2 |
| 96 | 0.311 | 3 | NT | | NT | |
| 97 | 0.056 | 3 | 0.287 | 4 | 92 | 2 |
| 98 | 0.122 | 3 | 1.309 | 2 | NT | |
| 99 | 0.059 | 3 | 0.233 | 3 | NT | |
| 100 | 0.104 | 3 | 0.682 | 3 | NT | |
| 101 | 0.048 | 3 | 0.464 | 2 | NT | |
| 102 | 0.269 | 3 | 0.749 | 1 | NT | |
| 103 | 0.104 | 4 | 0.392 | 2 | NT | |
| 104 | 0.101 | 4 | 0.517 | 2 | NT | |
| 105 | 0.123 | 4 | 0.732 | 2 | NT | |
| 106 | 0.245 | 4 | 1.086 | 1 | NT | |
| 107 | 0.564 | 3 | 1.870 | 1 | NT | |
| 108 | 0.100 | 3 | 0.367 | 3 | NT | |
| 109 | 0.421 | 2 | 0.908 | 1 | NT | |
| 110 | 0.554 | 2 | 1.589 | 1 | NT | |
| 111 | 0.416 | 3 | NT | NT | NT | |
| 112 | 0.234 | 3 | 0.895 | 2 | NT | |
| 113 | 0.350 | 2 | 1.622 | 1 | NT | |
| 114 | 0.156 | 3 | 1.512 | 2 | NT | |
| 115 | 0.142 | 3 | 0.415 | 2 | NT | |
| 116 | 0.018 | 2 | 0.172 | 3 | NT | |
| 117 | 0.275 | 3 | 0.769 | 2 | NT | |
| 118 | 0.243 | 3 | 0.727 | 2 | NT | |
| 119 | 0.470 | 3 | 4.208 | 2 | NT | |
| 120 | 0.788 | 3 | 2.337 | 1 | NT | |

Key:
¹ITK IC₅₀ values are presented as a geometric mean of count n
²IL-2 IC₅₀ values are presented as a geometric mean of count n
3TRKA % inhibition values are presented as an arithmetic mean of count n
NT means not tested

1. A compound of formula (I) 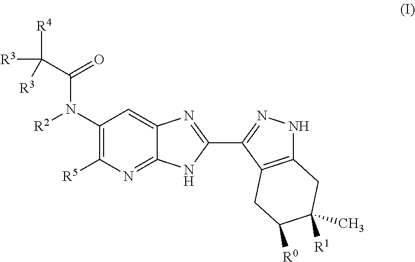
or a pharmaceutically acceptable salt thereof, wherein:
R⁰ is H and R¹ is methyl; or R⁰, R¹ and the carbon atoms to which they are attached form cyclopropyl, optionally gem-difluoro substituted;
R² is H, (C₁-C₄)alkyl, hydroxy(C₁-C₄)alkyl or (C₁-C₄)alkyl substituted by one, two or three F;
each R³ is independently H, F or (C₁-C₄)alkyl; or
both R³ taken together with the carbon atom to which they are attached form:
(C₄-C₇)cycloalkyl;
(C₄-C₇)cycloalkyl substituted by one, two or three F;
(C₄-C₇)cycloalkyl substituted by one or two R⁶;
(C₅-C₇)bicycloalkyl;
(C₅-C₇)bicycloalkyl substituted by one or two F;
C-linked 6-membered saturated heterocycloalkyl containing one N or one O, optionally substituted by oxo; or
C-linked 6-membered saturated heterobicycloalkyl containing one N or one O, optionally substituted by oxo;
R⁴ is H; F; (C₁-C₆)alkyl, optionally substituted by one, two or three F; —NR⁷R⁸; (C₄-C₇)cycloalkyl; C-linked 6-membered saturated heterocycloalkyl containing one N or one O, wherein said heterocycloalkyl is optionally substituted by oxo; N-linked 6-, 7- or 8-membered saturated heterocycloalkyl containing one N and optionally one O, wherein said heterocyclalkyl is optionally substituted by oxo; N-linked 6-, 7- or 8-membered saturated heterobicycloalkyl containing one N and optionally one O, wherein said heterobicyloalkyl is optionally substituted by oxo; or N-linked 5- or 6-membered heteroaryl containing one or two N;
R⁵ is H; halogen; (C₁-C₆)alkyl; (C₁-C₆)alkoxy; or (C₁-C₆)alkoxy substituted by (C₁-C₄)alkoxy;
each R⁶ is independently (C₁-C₃)alkyl;
R⁷ is (C₁-C₆)alkyl; and
R⁸ is (C₁-C₆)alkyl or —C(O)(C₁-C₆)alkyl.

2. The compound or a pharmaceutically acceptable salt thereof according to claim 1 of Formulae (Ia) or (Ib)

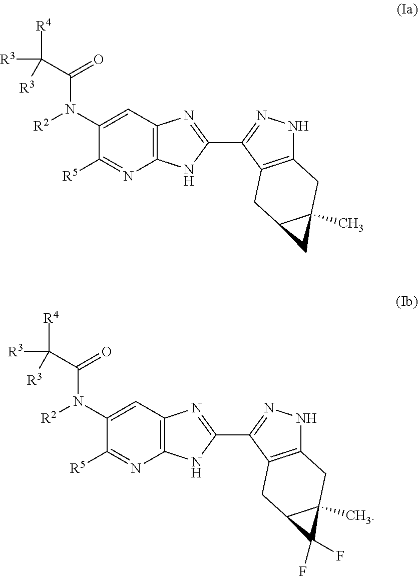

3. The compound or a pharmaceutically acceptable salt thereof according to claim 2 of Formulae (Ia).

4. The compound or a pharmaceutically acceptable salt thereof according to claim 1, wherein R² is H, methyl, ethyl or hydroxyethyl.

5. The compound or a pharmaceutically acceptable salt thereof according to claim 4, wherein R² is methyl.

6. The compound or a pharmaceutically acceptable salt thereof according to claim 1, wherein each R³ is independently H, F or (C₁-C₃)alkyl.

7. The compound or a pharmaceutically acceptable salt thereof according to claim 6, wherein each R³ is F.

8. The compound or a pharmaceutically acceptable salt thereof according to claim 6, wherein one R³ is H and the other R³ is methyl.

9. The compound or a pharmaceutically acceptable salt thereof according to claim 8, of Formula (Ic)

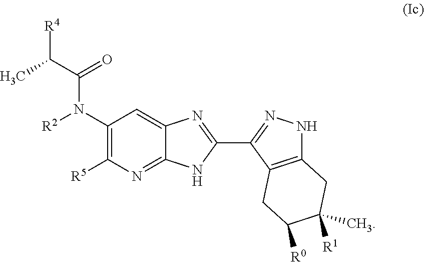

10. The compound or a pharmaceutically acceptable salt thereof according to claim 1, wherein both R³ taken together with the carbon atom to which they are attached form:
(C₄-C₇)cycloalkyl;
(C₄-C₇)cycloalkyl substituted by one, two or three F;
(C₄-C₇)cycloalkyl substituted by one or two R⁶;
(C₅-C₇)bicycloalkyl;
(C₅-C₇)bicycloalkyl substituted by one or two F;
C-linked 6-membered saturated heterocycloalkyl containing one N or one O, optionally substituted by oxo; or
C-linked 6-membered saturated heterobicycloalkyl containing one N or one O, optionally substituted by oxo.

11. The compound or a pharmaceutically acceptable salt thereof according to claim 1, wherein R⁴ is H; F; (C₁-C₄)alkyl, optionally substituted by one or two F; or —NR⁷R⁸.

12. The compound or a pharmaceutically acceptable salt thereof according to claim 1, wherein R⁴ is (C₄-C₇)cycloalkyl; C-linked 6 membered saturated heterocycloalkyl containing one O; N-linked 6-, 7- or 8-membered saturated heterocycloalkyl containing one N and optionally one O, wherein said heterocyclalkyl is optionally substituted by oxo; N-linked 6-, 7- or 8-membered saturated heterobicycloalkyl containing one N and optionally one O; or N-linked 5-membered heteroaryl containing two N.

13. The compound or a pharmaceutically acceptable salt thereof according to claim 12, wherein R⁴ is C-linked 6-membered saturated heterocycloalkyl containing one O.

14. The compound or a pharmaceutically acceptable salt thereof according to claim 1, wherein R⁵ is H, F, methyl, —OCH₃ or —OC₂H₅OCH₃.

15. The compound or a pharmaceutically acceptable salt thereof according to claim 14, wherein R⁵ is H.

16. A pharmaceutical composition comprising a compound formula (I) 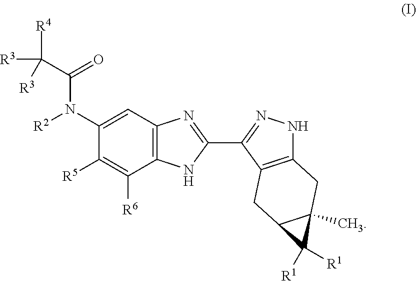
or a pharmaceutically acceptable salt thereof, wherein:
each R¹ is independently H or F:
R² is H or (C₁-C₄)alkyl;
each R³ is independently H, F or (C₁-C₄)alkyl; or
both R³ taken together with the carbon atom to which they are attached form:
a (C₃-C₆)cycloalkyl, optionally substituted by one or two F:
a (C₆-C₇)bicycloalkyl, optionally substituted by one or two F or;
a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O (with the proviso that R⁴ is not morpholinyl) wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F:
R⁷ is (C₁-C₆)alkyl; and
R⁸ is (C₁-C₆)alkyl or —C(O)(C₁-C₆)alkyl, and a pharmaceutically acceptable excipient.

17. The pharmaceutical composition according to claim 16, further comprising an additional therapeutic agents.

18-21. (canceled)

22. A method of treating a disorder in a human or animal for which an ITK inhibitor is indicated, comprising administering to said human or animal a therapeutically effective amount of a compound or formula (I) 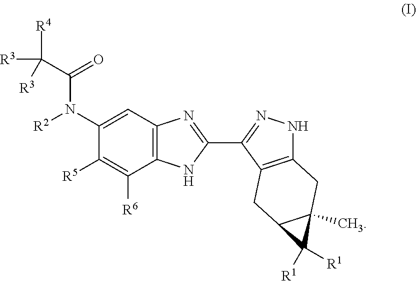
or a pharmaceutically acceptable salt thereof, wherein:
each R¹ is independently H or F:
R² is H or (C₁-C₄)alkyl;
each R³ is independently H, F or (C₁-C₄)alkyl; or
both R³ taken together with the carbon atom to which they are attached form:
a (C₃-C₆)cycloalkyl, optionally substituted by one or two F:
a (C₆-C₇)bicycloalkyl, optionally substituted by one or two F or;
a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O (with the proviso that R⁴ is not morpholinyl) wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F:
R⁷ is (C₁-C₆)alkyl; and
R⁸ is (C₁-C₆)alkyl or —C(O)(C₁-C₆)alkyl.

23. The method of claim 22, wherein the disorder is skin disorder.

24. The method of claim 23, wherein the skin disorder is atopic dermatitis.
